(19) United States
(12) Patent Application Publication
(10) Pub. No.: US 2022/0296594 A1
(43) Pub. Date: Sep. 22, 2022

(54) POTENTIATORS OF ANTIMICROBIAL AND/OR ANTIVIRAL AGENTS

(71) Applicants: Roby Bhattacharyya, Boston, MA (US); MASSACHUSETTS INSTITUTE OF TECHNOLOGY, Cambridge, MA (US); THE GENERAL HOSPITAL CORPORATION, Boston, MA (US)

(72) Inventors: Jason H. Yang, Cambridge, MA (US); James J. Collins, Cambridge, MA (US); Miguel A. Alcantar, Cambridge, MA (US); Roby Bhattacharyya, Boston, MA (US)

(73) Assignees: MASSACHUSETTS INSTITUTE OF TECHNOLOGY, Cambridge, MA (US); THE GENERAL HOSPITAL CORPORATION, Boston, MA (US)

(21) Appl. No.: 17/608,919

(22) PCT Filed: May 7, 2020
(86) PCT No.: PCT/US20/31873

§ 371 Date: Nov. 4, 2021

Related U.S. Application Data
(60) Provisional application No. 62/845,053, filed on May 8, 2019.

(51) Int. Cl. A61K 31/505; A61P 31/04; A61K 38/17; A61P 31/16; A61K 9/00; A61K 31/397

(52) U.S. Cl. CPC A61K 31/505; A61P 31/04; A61K 38/1729; A61P 31/16; A61K 9/0024; A61K 31/397

(57) ABSTRACT

The present disclosure provides compositions (e.g., pyrimidines) and methods capable of potentiating the effects of antimicrobial agents and/or antiviral agents against bacterial infections and/or viral infections, respectively. Methods of sensitizing bacteria to antimicrobial agents and/or antiviral agents, as well as pharmaceutical compositions and therapeutic/prophylactic methods directed at microbial infections and/or viral infections are also provided.

### CROSS REFERENCE TO RELATED APPLICATION

This application claims the benefit of U.S. Provisional Application No. 62/845,053, filed May 8, 2019, entitled “Potentiators of Antimicrobial Agents,” the entire contents of which are incorporated herein by reference.

### STATEMENT REGARDING FEDERALLY SPONSORED RESEARCH

This invention was made with government support under Grant No. K99-GM118907 awarded by the National Institutes of Health, Grant No. 1122374 awarded by the National Science Foundation and under Grant No. HDTRA1-15-1-0051 awarded by the Department of Defense. The government has certain rights in the invention.

### FIELD OF THE INVENTION

The invention relates generally to methods and compositions for potentiating antimicrobial agent efficacy.

### BACKGROUND OF THE INVENTION

Therapeutically effective use of antibiotics, a cornerstone of modern medicine, is threatened by the increasing prevalence of antibiotic-resistant and/or antibiotic-tolerant microbes. A need exists for compositions and/or improved approaches that can potentiate the efficacy of antimicrobial agents, both in vitro and in vivo.

### BRIEF SUMMARY OF THE INVENTION

The current disclosure relates, at least in part, to compositions and methods for potentiating the killing efficacy (lethality) of an antimicrobial agent (e.g., an antibiotic or antimicrobial peptide) against a target microbe, by sensitizing the target microbe to antimicrobial agent contact and/or treatment. In particular, an approach of contacting target bacteria (including, e.g., antibiotic-resistant bacteria and/or antibiotic-tolerant bacteria) with a pyrimidine compound together with an antibiotic was identified as potentiating the antimicrobial activity of a number of tested antibiotics.

In one aspect, the instant disclosure provides a pharmaceutical composition that includes (a) a pyrimidine; (b) an antimicrobial agent and/or an antiviral agent for non-chemotherapeutic use, or a pharmaceutically acceptable salt thereof, and a pharmaceutically acceptable carrier.

In certain embodiments, the pyrimidine is uracil, uridine, thymine, thymidine, cytosine and/or cytidine.

In some embodiments, the antimicrobial agent is bactericidal.

In certain embodiments, the antimicrobial agent is an antibiotic or an antimicrobial peptide.

In some embodiments, the antibiotic is a β-lactam antibiotic, an aminoglycoside antibiotic a quinolone antibiotic, a rifamycin (e.g., rifampicin), nitrofurantoin, metronidazole, trimethoprim, and/or a sulfonamide (e.g., sulfamethoxazole), or a salt, analog or derivative thereof.

In one embodiment, the β-lactam antibiotic is a penicillin derivative (e.g., Benzathine penicillin (benzathine & benzylpenicillin), Benzylpenicillin (penicillin G), Phenoxymethylpenicillin (penicillin V), Procaine penicillin (procaine & benzylpenicillin), Pheneticillin, Cloxacillin. Dicloxacillin, Flucloxacillin, Methicillin, Nafcillin, Oxacillin, Temocillin, Amoxicillin, Ampicillin, Mecillinam, Carbenicillin, Ticarcillin, Azlocillin, Mezlocillin, and Piperacillin); a cephalosporin (e.g., Cefazolin, Cephalexin, Cephalosporin C, Cephalothin, Cefaclor, Cefamandole, Cefuroxime, Cefotetan, Cefoxitin, Cefixime, Cefotaxime, Cefpodoxime, Ceftazidime. Ceftriaxone, Cefepime, Cefpirome, and Ceftaroline); a monobactam (e.g., Aztreonam, Tigemonam, Nocardicin A, and Tabtoxinine β-lactam); or a carbapenem or penem (e.g., Biapenem, Doripenem, Ertapenem, Faropenem, Imipenem, Meropenem, Panipenem, Razupenem, Tebipenem, and Thienamycin).

In another embodiment, the aminoglycoside antibiotic is gentamicin, streptomycin, kanamycin A, tobramycin, neomycin B, neomycin C, framycetin, paromomycin, ribostamycin, amikacin, arbekacin, bekanamycin (kanamycin B), dibekacin, spectinomycin, hygromycin B, paromomycin sulfate, netilmicin, sisomicin, isepamicin, verdamicin, astromicin, neamine, ribostamycin, or paromomycinlividomycin.

In certain embodiments, the quinolone antibiotic is ciprofloxacin, garenoxacin, gatifloxacin, gemifloxacin, levofloxacin, moxifloxacin, fleroxacin, lomefloxacin, nadifloxacin, norfloxacin, ofloxacin, pefloxacin, rufloxacin, balofloxacin, grepafloxacin, pazufloxacin, sparfloxacin, temafloxacin, clinafloxacin, sitafloxacin, prulifloxacin, besifloxacin, delafloxacin, danofloxacin, difloxacin, enrofloxacin, ibafloxacin, marbofloxacin, orbifloxacin, or sarafloxacin.

In some embodiments, the antimicrobial peptide is Bacitracin, Boceprevir, Dalbavancin, Daptomycin, Enfuvirtide, Oritavancin, Teicoplanin, Telaprevir, Telavancin, Vancomycin, or Guavanin 2.

In one embodiment, the pharmaceutical composition further includes a β-lactamase inhibitor, optionally sulbactam, tebipenem, a Boron based transition state inhibitor (e.g., Ec19), clavulanic acid, tazobactam, avibactam or relebactam.

In certain embodiments, the antibiotic is present in an amount between 0.1 g and 2.0 g.

In some embodiments, the antiviral agent is Abacavir (use for HIV), Acyclovir (Aciclovir—use for herpes e.g. Chicken pox), Adefovir (use for chronic Hepatitis B), Amantadine (use for influenza), Ampligen, Amprenavir (Agenerase—Use for inhibition of HIV), Arbidol, Atazanavir, Atripla (fixed dose drug), Balavir. Baloxavir marboxil (Xofluza), Biktarvy, Boceprevir (Victrelis), Cidofovir, Cobicistat (Tybost), Combivir (fixed dose drug), Daclatasvir (Daklinza), Darunavir, Delavirdine, Descovy, Didanosine, Docosanol, Dolutegravir, Doravirine (Pifeltro), Ecoliever, Edoxudine, Efavirenz, Elvitegravir, Emtricitabine, Enfuvirtide, Entecavir, Etravirine (Intelence), Famciclovir, Fixed dose combination (antiretroviral), Fomivirsen, Fosamprenavir, Foscarnet, Fosfonet, Fusion inhibitor, Ganciclovir (Cytovene), Ibacitabine, Ibalizumab (Trogarzo), Idoxuridine, Imiquimod, Imunovir, Indinavir, Inosine, Integrase inhibitor, Interferon type I, Interferon type II, Interferon type III, Interferon, Lamivudine, Letermovir (Prevymis), Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Peginterferon alfa-2b, Penciclovir, Peramivir (Rapivab), Pleconaril, Podophyllotoxin, Protease inhibitor (pharmacology), Pyramidine, Raltegravir, Remdesivir, Reverse transcriptase inhibitor, Ribavirin, Rilpivirine (Edurant), Rimantadine, Ritonavir, Saquinavir, Simeprevir (Olysio), Sofosbuvir, Stavudine, Synergistic enhancer (antiretroviral), Telaprevir, Telbivudine (Tyzeka), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza) and/or Zidovudine.

In one embodiment, the pyrimidine is provided in an amount sufficient to potentiate the rate at which the antimicrobial agent kills a target population of bacteria by at least 10%, as compared to an appropriate control (e.g., a control involving administration of the antimicrobial agent in the absence of the pyrimidine compound).

Another aspect of the disclosure provides a method for sensitizing a bacteria to an antimicrobial agent, involving contacting the bacteria with a pyrimidine, thereby sensitizing the bacteria to the antimicrobial agent.

In one embodiment, the bacteria exhibits resistance or tolerance to the antimicrobial agent.

In certain embodiments, the bacteria is an Escherichia coli, Klebsiella, Staphylococcus, Pseudomonas, Acinetobacter, Enterococcus, Enterobacter or Mycobacteria. Optionally, the Klebsiella is a Klebsiella pneumoniae, the Staphylococcus is a Staphylococcus aureus, the Pseudomonas is a Pseudomonas aeruginosa, the Acinetobacter is an Acinetobacter baumannii, the Enterococcus is an Enterococcus faecium or an Enterococcus faecalis, or the Mycobacteria is a Mycobacterium smegmatis or a Mycobacterium tuberculosis.

In some embodiments, the method also involves contacting the bacteria with a β-lactamase inhibitor.

An additional aspect of the disclosure provides a method for treating or preventing a bacterial infection in a subject, the method involving administering a pharmaceutical composition of the disclosure to a subject having or at risk of developing a bacterial infection, thereby treating or preventing the bacterial infection in the subject.

In one embodiment, the subject is a mammal. Optionally, the subject is a human.

In certain embodiments, the bacterial infection is a bacteremia.

In another embodiment, the bacterial infection is an antibiotic resistant or antibiotic tolerant bacterial infection.

In certain embodiments, the pharmaceutical composition is administered by injection. Optionally, the pharmaceutical composition is administered by intravenous injection.

In one embodiment, the bacterial infection is a localized bacterial infection. Optionally, the localized bacterial infection is a lung infection.

In some embodiments, the pharmaceutical composition is administered by aerosolization.

In certain embodiments, the pharmaceutical composition is administered topically.

Another aspect of the disclosure provides a kit that includes a pyrimidine, an antimicrobial agent and/or an antiviral agent for non-chemotherapeutic use, and instructions for its use.

In certain embodiments, the kit further includes an agent for measuring prpp accumulation and/or an agent for measuring the lethality (optionally, the increased lethality) of the antimicrobial agent and/or an antiviral agent against a target microbe and/or target virus.

A further aspect of the instant disclosure provides a method for treating or preventing a viral and/or microbial infection in a subject involving administering a pharmaceutical composition of the instant disclosure to a subject having or at risk of developing a viral and/or microbial infection, thereby treating or preventing the viral and/or microbial infection in the subject.

In certain embodiments, the viral and/or microbial infection is a lung infection. Optionally, the lung infection is a pneumonia.

In one embodiment, the lung infection is a COVID-19-related lung infection.

In some embodiments, the subject has a viral infection. Optionally, the subject has an influenza, rhinovirus and/or coronavirus infection. Optionally, the subject has a coronavirus infection. Optionally, the coronavirus infection is COVID-19.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

The term “infection” as used herein includes presence of a microbe (e.g., bacteria) or a virus, in or on a subject, which, if its growth were inhibited or if killing and/or clearing of the microbe or virus from a site of infection and/or subject were to occur, would result in a benefit to the subject. The term “infection” therefore refers to any undesirable form of microbe (e.g., bacteria) or virus that is present on or in a subject. As such, the term “infection” in addition to referring to the presence of microbe (e.g., bacteria) or virus also refers to normal flora, which are not desirable. The term “infection” includes infection caused by a microbe (e.g., bacteria) or virus.

The term “treat”, “treating” or “treatment” as used herein refers to administering a medicament, including a pharmaceutical composition, or one or more pharmaceutically active ingredients, for prophylactic and/or therapeutic purposes. The term “prophylactic treatment” refers to treating a subject who is not yet infected, but who is susceptible to, or otherwise at a risk of infection. The term “therapeutic treatment” refers to administering treatment to a subject already suffering from infection. The term “treat”. “treating” or “treatment” as used herein also refers to administering compositions or one or more of pharmaceutically active ingredients discussed herein, with or without additional pharmaceutically active or inert ingredients, in order to: (i) reduce or eliminate either a bacterial or viral infection or one or more symptoms of the bacterial or viral infection, or (ii) retard the progression of a bacterial or viral infection or of one or more symptoms of the bacterial or viral infection, or (iii) reduce the severity of a bacterial or viral infection or of one or more symptoms of the bacterial or viral infection, or (iv) suppress the clinical manifestation of a bacterial or viral infection, or (v) suppress the manifestation of adverse symptoms of the bacterial or viral infection.

The term “pharmaceutically effective amount” or “therapeutically effective amount” or “effective amount” as used herein refers to an amount, which has a therapeutic effect or is the amount required to produce a therapeutic effect in a subject. For example, a therapeutically or pharmaceutically effective amount of an antibiotic or a pharmaceutical composition is the amount of the antibiotic or the pharmaceutical composition required to produce a desired therapeutic effect as may be judged by clinical trial results, model animal infection studies, and/or in vitro studies (e.g., in agar or broth media). The pharmaceutically effective amount depends on several factors, including but not limited to, the microorganism (e.g., bacteria) involved, characteristics of the subject (for example height, weight, sex, age and medical history), severity of infection and the particular type of the antibiotic used. For prophylactic treatments, a therapeutically or prophylactically effective amount is that amount which would be effective to prevent a microbial (e.g. bacterial) infection.

The term “administration” or “administering” includes delivery of a composition or one or more pharmaceutically active ingredients to a subject, including for example, by any appropriate methods, which serves to deliver the composition or its active ingredients or other pharmaceutically active ingredients to the site of the infection. The method of administration may vary depending on various factors, such as for example, the components of the pharmaceutical composition or the type/nature of the pharmaceutically active or inert ingredients, the site of the potential or actual infection, the microorganism involved, severity of the infection, age and physical condition of the subject and a like. Some non-limiting examples of ways to administer a composition or a pharmaceutically active ingredient to a subject according to this invention includes oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop, ear drop or mouthwash. In case of a pharmaceutical composition that comprises more than one ingredient (active or inert), one of way of administering such composition is by admixing the ingredients (e.g. in the form of a suitable unit dosage form such as tablet, capsule, solution, powder and a like) and then administering the dosage form. Alternatively, the ingredients may also be administered separately (simultaneously or one after the other) as long as these ingredients reach beneficial therapeutic levels such that the composition as a whole provides a synergistic and/or desired effect.

An “active agent” refers to an agent that either possesses antimicrobial activity, i.e., inhibits growth and/or survival of a microorganism, or that potentiates an agent that possesses antimicrobial activity.

The term “growth” as used herein refers to a growth of one or more microorganisms and includes reproduction or population expansion of the microorganism (e.g., bacteria). The term also includes maintenance of on-going metabolic processes of a microorganism, including processes that keep the microorganism alive.

The term, “effectiveness” as used herein refers to ability of a treatment or a composition or one or more pharmaceutically active ingredients to produce a desired biological effect in a subject. For example, the term “antibiotic effectiveness” of a composition or a beta-lactam antibiotic refers to the ability of the composition or the beta-lactam antibiotic to prevent or treat the microbial (e.g., bacterial) infection in a subject.

The term “synergistic” or “synergy” as used herein refers to the interaction of two or more agents so that their combined effect is greater than their individual effects.

As used herein, the term “antimicrobial agent” refers to any compound known to one of ordinary skill in the art that will inhibit or reduce the growth of, or kill, one or more microorganisms, including bacterial species and fungal species.

In certain embodiments, the term “antibiotic” refers to any substance, compound or a combination of substances or a combination of compounds capable of: (i) inhibiting, reducing or preventing growth of bacteria; (ii) inhibiting or reducing ability of a bacteria to produce infection in a subject; or (iii) inhibiting or reducing ability of bacteria to multiply or remain infective in the environment. The term “antibiotic” also refers to compounds capable of decreasing infectivity or virulence of bacteria. Many antibacterial compounds are relatively small molecules with a molecular weight of less than 2000 atomic mass units. The term “antibiotic” includes semi-synthetic modifications of various natural compounds, such as, for example, the beta-lactam antibiotics, which include penicillins (produced by fungi in the genus Penicillium), the cephalosporins, and the carbapenems. Accordingly, the term “antibiotic” includes, but is not limited to, aminoglycosides (e.g., gentamicin, streptomycin, kanamycin), β-lactams (e.g., penicillins and cephalosporins), vancomycins, bacitracins, macrolides (e.g., erythromycins), lincosamides (e.g., clindomycin), chloramphenicols, tetracyclines, amphotericins, cefazolins, clindamycins, mupirocins, sulfonamides and trimethoprim, rifampicins, metronidazoles, quinolones, novobiocins, polymixins, gramicidins, or any salts or variants thereof.

The term “pyrimidine compound” as used herein refers to a compound that possesses a pyrimidine ring system. The pyrimidine ring system has the following structure:

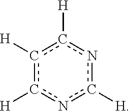

By “control” or “reference” is meant a standard of comparison. Methods to select and test control samples are within the ability of those in the art. Determination of statistical significance is within the ability of those skilled in the art, e.g., the number of standard deviations from the mean that constitute a positive result.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the term “tissue” is intended to mean an aggregation of cells, and, optionally, intercellular matter. Typically the cells in a tissue are not free floating in solution and instead are attached to each other to form a multicellular structure. Exemplary tissue types include muscle, nerve, epidermal and connective tissues.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIGS. 1A to 1C depict schematics of the instant “white-box” machine learning approach for revealing metabolic mechanisms of antibiotic lethality, as disclosed herein. FIG. 1A shows a schematic elucidating how machine learning activities are typically comprised of three parts: input data (blue), output data (red), and a predictive model trained to compute output data from input data (purple). FIG. 1B shows a schematic of an overall framework for white-box machine learning. Input screening perturbations (e.g., metabolite conditions; gray) are first transformed into enriched biological network states by prospective network modeling (e.g., metabolic fluxes; blue). These network simulations are then used as machine learning inputs to train a predictive model (purple), revealing mechanisms underlying the output data (e.g., antibiotic lethality measurements; red). Since biological networks are mechanistically constructed, features comprising the predictive models trained by machine learning are, by definition, mechanistically causal. FIG. 1C shows an exemplary overall workflow process. E. coli MG1655 cells were treated with three bactericidal antibiotics at 13 different concentrations. Antibiotic IC₅₀s were quantified following supplementation with 206 diverse metabolites and normalized by their on-plate controls. Metabolic network states corresponding to each metabolite were prospectively simulated using the iJO1366 model of E. coli metabolism (Orth et al., 2011). For each antibiotic, changes in IC₅₀ were regressed on the simulated fluxes and pathway mechanisms were identified by hypergeometric testing on metabolic pathways curated by Ecocyc (Keseler et al., 2017). Identified pathways were validated experimentally.

FIGS. 24 and 2B show how exogenous metabolites exert pathway-specific effects on antibiotic lethality. FIG. 2A depicts an overall experimental design for measuring metabolite effects on antibiotic lethality. Overnight cultures of E. coli MG1655 were inoculated into MOPS minimal medium, grown to early exponential phase, and back-diluted to OD₆₀₀=0.1. Cells were dispensed into Biolog phenotype microarray plates (PMs) 14 (Bochner, 2009) with different concentrations of ampicillin (AMP), ciprofloxacin (CIP) or gentamicin (GENT) added. OD₆₀₀ was measured after 4 hours of incubation at 37° C. and 900 rpm shaking. Antibiotic IC₅₀s were estimated for each antibiotic-metabolite combination. FIG. 2B presents antibiotic IC₅₀ responses to metabolite supplementation. Metabolically-induced sensitivity profiles differ by antibiotic, but several metabolites commonly protect (red) or sensitize (blue) cells to multiple antibiotics. Carbon metabolites were screened using Biolog PMs 1 and 2; nitrogen metabolites were screened using Biolog PM 3; phosphorus and sulfur metabolites were screened using Biolog PM 4. Data are represented as mean from n≥3 independent biological replicates.

FIG. 3 elucidates how white-box machine learning reveals known and new antibiotic mechanisms of action. Pathway scores for metabolic pathways identified by white-box machine learning are represented by heat map. Identified pathways include several central carbon metabolism and nucleotide biosynthesis pathways, and these cluster into three groups, based on pathway score. Central metabolism pathways primarily exhibit similar pathway directionality for ampicillin (AMP), ciprofloxacin (CIP), gentamicin (GENT), while purine biosynthesis pathways exhibit different pathway score directionality for GENT from AMP or CIP. Pathway scores were computed for each antibiotic by log-transforming the average regression coefficient for all non-zero reactions annotated in a given pathway.

FIGS. 4A to 4E show how purine biosynthesis participates in antibiotic lethality. FIG. 4A presents the purine biosynthesis pathway. Purine biosynthesis begins with phosphoribosyl pyrophosphate (prpp) and contains several ATP consuming steps (purple). FIG. 4B displays graphs which demonstrate antibiotic lethality in purine biosynthesis deletion mutants. Genetic inhibition of purine biosynthesis by purD (glycinamide ribonucleotide synthetase), purE (M-carboxyaminoimidazole ribonucleotide mutase), purK (5-(carboxyamino)imidazole ribonucleotide synthase), or purM (phosphoribosylformylglycinamide cyclo-ligase) deletion decreases ampicillin (AMP) and ciprofloxacin (CIP) lethality, but increases gentamicin (GENT) lethality. FIG. 4C presents graphs which show antibiotic lethality following biochemical inhibition of purine biosynthesis. Biochemical inhibition of PurF (amidophosphoribosyltransferase) by 6-mercaptopurine (6-MP) decreases AMP and CIP lethality, but increases GENT lethality. FIG. 4D presents graphs which show antibiotic lethality in a glyA (serine hydroxymethyltransferase) deletion mutant. Genetic inhibition of glycine (gly) and N¹⁰-formyl-tetrahydrofolate (10fthf) by glyA deletion decreases AMP and CIP lethality, but increases GENT lethality. FIG. 4E presents graphs which show antibiotic lethality following enhanced purine biosynthesis. Substrate-level stimulation of purine biosynthesis with phosphoribosyl pyrophosphate (prpp) and glutamine (gln) supplementation increases AMP and CIP lethality, but decreases GENT lethality. Data are represented as mean±SEM from n≥3 independent biological replicates.

FIGS. 5A to 5C show how adenine limitation contributes to antibiotic lethality. FIG. 5A shows a schematic of feedback inhibition in the purine and pyrimidine biosynthesis pathways. Purine and pyrimidine biosynthesis auto-regulate through internal feedback inhibition by nucleotide end-products. FIG. 5B presents graphs that show antibiotic lethality following purine supplementation. Adenine supplementation (red) decreases ampicillin (AMP), ciprofloxacin (CIP) and gentamicin (GENT) lethality. FIG. 5C presents graphs of antibiotic lethality following pyrimidine supplementation. Uracil supplementation (dark blue) increases AMP, CIP and GENT lethality. Data are represented as mean±SEM from n=3 independent biological replicates.

FIGS. 6A to 6F show how adenine supplementation reduces ATP demand and central carbon metabolism activity. FIG. 6A presents bar graphs representing metabolic modeling predictions. Adenine supplementation decreases activity through purine biosynthesis, consequently decreasing ATP utilization by purine biosynthesis, central carbon metabolism and oxidative phosphorylation (see FIG. 11 below), in comparison to simulated control (CTL). E. coli metabolism under adenine (ADE) or uracil (URA) supplementation was simulated by parsimonious flux balance analysis (pFBA) in the iJO1366 metabolic model with exchange reactions for adenine or uracil opened, respectively. Nucleotide biosynthesis activity was computed by summing fluxes through reactions in the Purine and Pyrimidine Biosynthesis subsystem (left). ATP consumption was summed across all reactions in the Purine and Pyrimidine Biosynthesis and Nucleotide Salvage Pathway subsystems (center left). Central carbon metabolism activity was computed by summing fluxes through reactions in the Glycolysis and TCA Cycle subsystems (center right). Oxidative phosphorylation is proxied by the Succinate Dehydrogenase reaction (right): additional oxidative phosphorylation reactions are depicted in FIG. 11. All fluxes were normalized by the biomass objective function. FIG. 6B presents bar graphs depicting intracellular adenine or uracil concentrations following adenine or uracil supplementation. Intracellular metabolite concentrations were measured by targeted LC-MS/MS. FIG. 6C presents bar graphs of intracellular succinate or fumarate concentrations following adenine or uracil supplementation. Adenine supplementation increases intracellular succinate and decreases intracellular fumarate, consistent with model predictions for inhibited succinate dehydrogenase activity. FIG. 6D presents bar graphs of ATP synthesis following adenine or uracil supplementation. Metabolic modeling simulations predict a decrease in ATP synthesis following adenine supplementation (left), reported by the ATP Synthase reaction. Metabolomic measurements of intracellular ATP, ADP and AMP (FIG. 12B) reveal a similar decrease in adenylate energy charge following adenine supplementation (right). FIG. 6E presents bar graphs of NADPH/NADP⁺ and NADH/NAD⁺ ratios following adenine or uracil supplementation. Metabolomic measurements of intracellular NADPH, NADP⁺, NADH and NAD⁺ (see FIG. 12C below) reveal modest decreases in the NADPH/NADP⁺ ratio following adenine supplementation (left), indicating reduced anabolic metabolism. The NADH/NAD⁺ ratio is largely unchanged (right), indicating preserved catabolic metabolism. FIG. 6F presents graphs of cellular respiration following adenine or uracil supplementation during antibiotic treatment. Metabolic modeling simulations predict a decrease in oxygen consumption following adenine supplementation (left), reported by the Oxygen Exchange reaction. Adenine supplementation (red) reduces respiratory activity, while uracil (blue) increases respiratory activity. Changes in oxygen consumption rate following treatment with ampicillin (AMP), ciprofloxacin (CIP) or gentamicin (GENT) and adenine or uracil supplementation were measured using the Seahorse Extracellular Flux Analyzer. Data are represented as mean±SEM from n=3 independent biological replicates. Significance reported as FDR-corrected p-values in comparison with control: ¶: p≤0.1, *: p≤0.05, **: p≤0.01, ****: p≤0.0001.

FIG. 7 presents a schematic diagram that depicts how antibiotic-induced adenine limitation is modeled to induce purine biosynthesis, increasing ATP demand and driving central carbon metabolic activity. In addition to the lethal effects of inhibiting their primary targets, bactericidal antibiotics disrupt the nucleotide pool, depleting intracellular purines and inducing adenine limitation. Adenine limitation triggers purine biosynthesis, increasing ATP demand, which drives increased activity through central carbon metabolism and cellular respiration. Toxic metabolic byproducts generated by this increased metabolic activity damage DNA and exacerbate antibiotic-mediated killing. Futile cycles and other stress-induced phenomena may also elevate ATP demand.

FIG. 8 presents a pathway diagram depicting antibiotic reaction scores for purine biosynthesis (based on FIGS. 3 and 4A-4E above). Differences in purine biosynthesis pathway scores for ampicillin (AMP) and ciprofloxacin (CIP) from gentamicin (GENT) are primarily explained by early reactions in the purine biosynthesis pathway (gray box).

FIGS. 9A and 9B show that purine biosynthesis participates in antibiotic lethality (based on FIGS. 4A-4E above). FIG. 9A presents graphs which demonstrate antibiotic lethality in pyrimidine biosynthesis deletion mutants. Genetic inhibition of pyrimidine biosynthesis by pyrC (dihydroorotase) or pyrE (orotate phosphoribosyltransferase) deletion does not significantly change ampicillin (AMP), ciprofloxacin (CIP) or gentamicin (GENT) lethality.

FIG. 9B demonstrates that biochemical disruption of the folate cycle by trimethoprim (TRI) decreases AMP and CIP lethality, but increases GENT lethality. Data are represented as mean±SEM from n=3 independent biological replicates.

FIG. 10 shows that antibiotic stress rapidly disrupts intracellular nucleotide pools (based on FIGS. 5A-5C above). Purine nucleic acid bases (A: adenine, G: guanine) are depleted (red), while pyrimidine nucleic acid bases (C: cytosine, T: thymine, U: uracil) accumulate (blue) in E. coli cells treated with ampicillin (AMP), norfloxacin (NOR) or kanamycin (KAN). (Data reanalyzed from Belenky et al., 2015.)

FIG. 11 presents bar graphs reflecting model simulations which predict that exogenous adenine supplementation decreases oxidative phosphorylation (based on FIGS. 6A-6F above).

FIGS. 12A to 12E demonstrate that adenine or uracil supplementation alters energy currencies and central carbon metabolism (based on FIGS. 6A-6F above). FIG. 12A shows that exogenous adenine (red) or uracil (blue) supplementation does not significantly alter unstressed growth in MOPS minimal medium. FIG. 12B shows bar graphs of measured intracellular AMP, ADP and ATP concentrations following adenine (ADE) or uracil (URA) supplementation. FIG. 12C shows bar graphs of measured relative intracellular NADPH, NADP⁺, NADH and NAD⁺ concentrations following adenine or uracil supplementation. FIG. 12D shows bar graphs of intracellular tricarboxylic acid cycle metabolite concentrations following adenine or uracil supplementation. FIG. 12E presents a plot of cellular respiration (O₂ consumption rate) following adenine or uracil supplementation in the absence of antibiotic treatment. Data are represented as mean±SEM from n≥3 independent biological replicates. Significance reported as FDR-corrected p-values in comparison with control: ¶: p<0.1, *: p<0.05, **: p<0.01, ***: p<0.001.

FIG. 13 shows that a range of pyrimidine compounds—specifically, uracil, cytosine and uridine—exhibited a potentiating effect upon antibiotic lethality, across tested antibiotics ampicillin (AMP), ciprofloxacin (CIP) and gentamicin (GENT).

### DETAILED DESCRIPTION OF THE INVENTION

The present disclosure is directed, at least in part, to the discovery that pyrimidines can potentiate the microbial killing activity of antimicrobial agents. Specifically, pyrimidine supplementation has been identified herein as an effective means of increasing antibiotic lethality. The instant disclosure therefore provides compositions capable of potentiating the effects of antimicrobial agents against microbial infections, including those that have developed, or that are capable of developing, resistance and/or tolerance to an antimicrobial agent, absent contact with the compositions of the disclosure. Methods of sensitization (via contact with a pyrimidine compound) of a microbe—including, e.g., an antibiotic resistant and/or antibiotic tolerant bacteria—to an antimicrobial agent are therefore provided, as are pharmaceutical compositions and therapeutic/prophylactic methods directed at potentiating antimicrobial agent killing efficacy against a targeted microbe.

The instant disclosure addresses the problem of antibiotic treatment failure by potentiating the bactericidal activity of antimicrobial agents. This problem has been addressed in the past by either antibiotic combination therapy or by using central carbon metabolism metabolites as adjuvants. Such previous approaches are limited by PK/PD considerations, off-target effects and bioavailability of each constituent therapeutic. The instant disclosure overcomes each of these limitations as representative pyrimidine compounds (e.g., uracil) exhibit good plasma PK/PD and do not exert toxic effects on humans at relevant concentration ranges.

Identification of pyrimidine supplementation as an effective means of increasing antimicrobial agent lethality involved implementation of a “white box” machine learning technique. While current machine learning techniques enable robust association of biological signals with measured phenotypes, these approaches are incapable of identifying causal relationships. Certain aspects of the instant disclosure have herein described an integrated white-box biochemical screening, network modeling and machine learning approach for revealing causal mechanisms and have initially applied this approach towards understanding antibiotic efficacy. In brief, diverse metabolites have been counter-screened against bactericidal antibiotics in Escherichia coli and corresponding Escherichia coli metabolic states have been simulated using a genome-scale metabolic network model. Regression of the measured screening data on model simulations has revealed herein that purine biosynthesis participates in antibiotic lethality, which has been validated experimentally. Antibiotic-induced adenine limitation has been further demonstrated herein to increase ATP demand, which elevates central carbon metabolism activity and oxygen consumption, enhancing the killing effects of antibiotics. The instant disclosure therefore demonstrates how prospective network modeling can couple with machine learning to identify complex causal mechanisms underlying drug efficacy.

Recent advances in high-throughput experimental technologies and data analyses have enabled unprecedented observation, quantification and association of biological signals with cellular phenotypes. Data-driven machine learning activities are poised to transform biological discovery and the treatment of human disease (Camacho et al., 2018; Wainberg et al., 2018; Webb, 2018; Yu et al., 2018a); however, existing techniques for extracting biological information from large datasets frequently encode relationships between perturbation and phenotype in opaque “black-boxes” that are mechanistically uninterpretable, and consequently can only identify correlations as opposed to causal relationships (Ching et al., 2018). In natural systems, biological molecules are biochemically organized in networks of complex interactions underlying observable phenotypes; biological network models may therefore harbor the potential to provide mechanistic structure to machine learning activities, yielding transparent “white-box” causal insights (Camacho et al., 2018; Yu et al., 2018b).

Chemical and genetic screens are workhorses in modern drug discovery, but frequently suffer from poor (1-3%) hit rates (Roses, 2008). Such low hit rates often underpower the bioinformatic analyses used for causal inference due to limitations in biological information content. Experimentally validated network models possess the potential to expand the biological information content of sparse screening data; however, biological screening experiments are typically performed independently from network modeling activities, limiting subsequent analyses to either post hoc bioinformatic enrichment from screening hits or experimental validation of existing models. There is therefore a need to develop biological discovery approaches that integrate biochemical screens with network modeling and advanced data-analytical techniques, so as to enhance understanding of complex drug mechanisms (Camacho et al., 2018; Wainberg et al., 2018; Xie et al., 2017). In certain aspects, the instant disclosure has developed one such approach and has initially applied it towards understanding antibiotic mechanisms of action.

As noted above, antibiotic efficacy is threatened by the increasing burden of drug resistance, which is compounded by a diminished antimicrobial discovery pipeline (Brown and Wright, 2016). Although the primary targets and mechanisms of action for conventional antibiotics are well studied (Kohanski et al., 2010), there is growing appreciation that secondary processes such as altered metabolism actively participate in antibiotic efficacy (Yang et al., 2017a), and that extracellular metabolites may either potentiate (Allison et al., 2011: Meylan et al., 2017) or suppress (Yang et al., 2017b) the lethal activities of bactericidal antibiotics. While features of central metabolism (Kohanski et al., 2007) and cellular respiration (Gutierrez et al., 2017; Lobritz et al., 2015) are implicated in antibiotic lethality across diverse microbial species (Dwyer et al., 2015), the biological mechanisms underlying antibiotic-induced changes to metabolism (Belenky et al., 2015; Dwyer et al., 2014) remain unclear. Deeper understanding into how bacterial metabolism interfaces with antibiotic lethality has the potential to open new drug discovery paradigms (Bald et al., 2017; Murima et al., 2014), making antibiotic-induced cellular death physiology an attractive topic to investigate with white-box machine learning.

In certain aspects, the instant disclosure has integrated biochemical screening, network modeling and machine learning to form a white-box machine learning approach for revealing drug mechanisms of action. This approach has been applied herein towards elucidating metabolic mechanisms of action for bactericidal antibiotics. The instant approach has discovered that metabolic processes related to purine biosynthesis, driven by antibiotic-induced adenine limitation, participate in antibiotic lethality. Without wishing to be bound be theory, as demonstrated herein, adenine limitation increases ATP demand via purine biosynthesis, resulting in elevated central carbon metabolism activity and oxygen consumption, thereby enhancing the killing effects of antimicrobial agents. The instant disclosure has therefore demonstrated how network models can facilitate machine learning activities for biological discovery and provide insights into complex causal mechanisms underlying drug efficacy.

Advances in high-throughput experimental technologies and data science have stimulated considerable interest in the potential for artificial intelligence to transform biological discovery and healthcare (Gil et al., 2014; Topol, 2019; Webb, 2018; Yu et al., 2018a). Important for such pursuits will be the necessary transition from correlation-based machine learning to causality-based “machine reasoning” (Bottou, 2014). Identifying causal mechanisms by modern machine learning approaches is challenging due to the mechanistic inaccessibility of computationally derived, black-box associations between perturbations and phenotypes. In the instant disclosure, biological network models have been successfully employed to overcome this mechanistic uncertainty and to help uncover biological mechanisms (Camacho et al., 2018; Yu et al., 2018b).

Network modeling has long provided a foundation for systems biology (Ideker et al., 2001) and researchers are now beginning to integrate machine learning with retrospective network modeling for improving the fidelity of genotype-to-phenotype predictions (Ma et al., 2018). Such activities demonstrate how hierarchically organized prior knowledge can deconvolve complex biological data; however, these efforts rely on post hoc analyses of experimental data and can only perform inductive association of phenotypes with perturbations rather than deductive identification of the causal mechanisms driving phenotypes. This application presented a complementary approach, combining machine learning with prospective network modeling to infer biological mechanisms based on their combined information content.

The utility of how this approach can be integrated with biochemical screening and applied towards understanding mechanisms underlying antibiotic efficacy has been demonstrated herein. Antibiotics are conventionally understood to work by inhibiting processes involved in bacterial cell replication (Kohanski et al., 2010). However, recent work has shown that processes downstream of target inhibition, including bacterial metabolism, actively participate in antibiotic lethality (Cho et al., 2014; Dwyer et al., 2015; Gruber and Walker, 2018; Zhao and Drlica, 2014). An important knowledge gap has been in understanding the biological mechanisms underlying antibiotic-mediated changes in metabolism. The results obtained herein have indicated that altered metabolism resulting from bactericidal antibiotic treatment is driven, in part, by the increased ATP demand required to restore homeostasis to a disrupted nucleotide pool (Belenky et al., 2015). It is likely that antibiotic-induced insults to the nucleotide pool are further exacerbated by nucleotide oxidation (Fan et al., 2018; Foti et al., 2012; Gruber and Walker, 2018), resulting in a positive feedback loop of increased nucleotide biosynthesis, elevated central carbon metabolism and toxic metabolic byproduct generation that is lethally detrimental to the cell (see FIG. 7). Because nucleotide analogues are commonly used as FDA-approved anticancer and antiviral chemotherapeutics, the prospect of clinical assessment of such compounds for use as antimicrobial agents or adjuvants (El Zahed and Brown, 2018; Serpi et al., 2016) should be able to proceed expeditiously.

Adenine nucleotides have been previously described as important mediators of cellular homeostasis (Andersen and von Meyenburg, 1977; Chapman and Atkinson, 1977), universally coupling cellular metabolism, DNA/RNA replication, and other physiological processes. In the context of infection, adenylate metabolites such as ATP, ADP and adenosine are important components of the damage-associated molecular patterns used by the host to activate the immune system (Cekic and Linden, 2016). It was previously observed that adenine metabolites such as AMP accumulate at a site of infection during antibiotic treatment and, consistent with the data collected in the instant disclosure, can inhibit antibiotic lethality (Yang et al., 2017b). Given the results obtained in the current disclosure, it is likely that inter-patient differences in the concentrations of extracellular nucleotides contribute to variable antibiotic treatment outcomes for infection (Lee and Collins, 2011). Moreover, one of the surprising findings of the instant disclosure—that uracil potentiates antibiotic lethality (see FIG. 5)—indicates that pyrimidine nucleotides can act as antimicrobial adjuvants/potentiate antibiotic activity. Without wishing to be bound by theory, uracil supplementation is believed to act by stimulating the overall central carbon metabolism and respiration activity of bacterial pathogens. While uracil is not itself a central carbon metabolism intermediate, it appears to stimulate this metabolic activity without being directly catabolized as a substrate.

Evolution has optimized bacteria for efficient resource allocation under unstressed growth (Basan et al., 2015; Hui et al., 2015; Scott et al., 2014), and insults to the ATP pool and other energy currencies are sufficient for stimulating central carbon metabolism (Holm et al., 2010; Koebmann et al., 2002) and sensitizing cells to oxidative stress (Adolfsen and Brynildsen, 2015). Additionally, intracellular ATP and the adenylate energy charge are tightly regulated across the tree of life, and robustly maintained across environmental changes and cellular insults (Chapman and Atkinson, 1977). Under antibiotic stress, increases to ATP demand are likely to arise from multiple sources (Yang et al., 2017a). Consistent with these notions, pharmacological suppression of oxidative phosphorylation (Shetty and Dick, 2018) and metabolic conditions inhibiting intracellular ATP (Shan et al., 2017) have been described to protect cells against antibiotics, supporting a critical role for ATP dynamics in antibiotic-mediated lethality. Additionally, futile cycling in cell wall synthesis and degradation was recently reported to be a component of β-lactam lethality (Cho et al., 2014). The findings disclosed herein support a new, fundamental concept in understanding antibiotic death physiology—specifically, that stress-induced changes in ATP utilization and demand, as a homeostatic response, critically drive lethal metabolic alterations. Since antibiotic stress increases the abundance of central carbon metabolism intermediates (Belenky et al., 2015; Nandakumar et al., 2014) and TCA cycle protein expression (Babin et al., 2017), central carbon metabolism should now be explored as a target for antimicrobial drug discovery (Bald et al., 2017; Murima et al., 2014).

The growing, global crisis of antibiotic resistance has created a clear imperative for expanded efforts in antimicrobial drug discovery and investigations into bacterial cellular death physiology (Brown and Wright, 2016). As experimental and computational technologies mature, new techniques and resources are becoming available for studying the biological mechanisms underlying antibiotic responses in complex and dynamic environments (Certain et al., 2017; Dunphy and Papin, 2017; Mack et al., 2018; Yang et al., 2017a). While the work described here has specifically focused on bacterial metabolism, several other aspects of bacterial physiology are known to be relevant to antibiotic efficacy, including bacterial stress responses, DNA repair mechanisms, and macromolecular processes such as transcription and translation (Dwyer et al., 2015: Gruber and Walker, 2018; Yang et al., 2017a). Investigation into these other physiological systems will benefit from new and different modeling approaches (Carrera and Covert, 2015: Ma et al., 2018; Oberhardt et al., 2013: Yang et al., 2018), curated knowledge bases (Karr et al., 2012; Keseler et al., 2017; Monk et al., 2017), and screening innovations (French et al., 2018; French et al., 2016). Integration of such resources with machine learning is projected to advance antibiotic discovery by revealing novel mechanisms that can be targeted with next-generation adjuvants, boosting the existing antibiotic arsenal (Tyers and Wright, 2019).

As disclosed in certain aspects herein, white-box machine learning can be broadly extended across diverse biological systems and, as demonstrated herein, be impactful for revealing drug mechanisms of action for treating human diseases. For instance, cell metabolism is increasingly recognized as being important in cancer pathogenesis (Vander Heiden and DeBerardinis, 2017), and histidine metabolism was recently demonstrated to participate in the efficacy of some cancer therapeutics (Kanarek et al., 2018). Similar to the present work on antibiotics, cancer drugs may be counter-screened against a library of metabolites in human cancer cells and coupled with network simulations using models of human metabolism (Brunk et al., 2018) to discover metabolic mechanisms of action for existing cancer drugs. Insights gained by such an approach are expected to guide the design of cancer treatment regimens that account for a tumor's local metabolic microenvironment, leveraging metabolic perturbations to optimize treatment outcomes.

Moreover, the integrated screening-modeling-learning approach discussed herein is agnostic to the experimental datasets and network models used to train machine learning models. NIH Common Fund programs such as “Library of Integrated Network-Based Cellular Signatures” (LINCS) and “Big Data to Knowledge” are providing increasingly comprehensive measurements of cellular physiology in response to genetic or small molecule perturbations (Keenan et al., 2018). It is now contemplated that the above-described white-box machine learning approach can be extended to such datasets to reveal molecular mechanisms mediating cellular responses to biochemical stimuli. For instance, simulations can be performed on human signaling networks to transform LINCS small molecule perturbations into signaling network configurations, which can be utilized as input data to learn signaling mechanisms of epigenetic regulation from measured chromatin signatures (Litichevskiy et al., 2018). Similarly, prospective network simulations can be performed on gene regulatory networks to interpret CRISPR screening perturbations (Wang et al., 2014) and are likely to reveal transcriptional programs underlying screened phenotypes.

Finally, white-box machine learning is contemplated to be important for realizing the transformative promises of translational precision medicine activities such as NIH's “All of Us” research program. Simulations can be performed on biological networks curated in databases such as BioGRID (Stark et al., 2006) to transform human data from repositories such as the UK Biobank (Bycroft et al., 2018) into gene regulatory, signaling or metabolic network states customized for each individual patient in a diverse population. These customized network states may be applied as inputs to machine learning models to identify mechanistically interpretable biomarkers and molecular mechanisms of disease pathogenesis from relevant clinical metadata, using classification and regression techniques. Such analyses are expected to be impactful for treating human disease by enabling stratified, personalized treatment strategies based on an individual's gene regulatory, signaling or metabolic network state and by providing new targets for drug discovery programs (Yu et al., 2018a). Reaching such endpoints is predicted to require continued high-quality characterization of human specimens and curation of human biological networks. However, white-box machine learning is expected to reward such efforts with deep, new insights capable of enabling truly personalized medicine.

Effective treatment of bacterial infections is limited when bacteria are able to evade antibiotic action. Common mechanisms of antibiotic resistance are well understood in the art, and adjuvants and multidrug strategies targeting these resistance mechanisms are being developed and deployed clinically (Baym et al. Science 351, aad3292; Wright, G. D. Trends Microbiol. 24, 928; Drawz and Bonomo. Clin. Microbiol. Rev. 23, 160-201; Imamovic et al. Cell 172, 121-134).

Bacteria can also become tolerant to antibiotic treatment. Tolerance specifically refers to an inability of high concentrations of antibiotics—typically lethal concentrations that are above the growth-inhibitory threshold for a given strain—to kill bacteria. Tolerant bacterial infections are believed to contribute to recurrent infections which then take longer to treat, driving up treatment costs.

Various expressly contemplated components of certain compositions and methods of the instant disclosure are considered in additional detail below.

### Pyrimidine Compounds

In certain aspects, the present disclosure provides for use of pyrimidine compounds, optionally in combination with other agents, to potentiate the anti-microbial efficacy of antimicrobial agents (e.g., co-administered antibiotics). Pyrimidines are characterized by the pyrimidine ring system, which has the following structure:

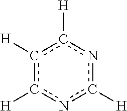

Pyrimidine is an aromatic heterocyclic organic compound similar to pyridine (Gilchrist, Thomas Lonsdale (1997). Heterocyclic chemistry. New York: Longman). One of the three diazines (six-membered heterocyclics with two nitrogen atoms in the ring), it has the nitrogen atoms at positions 1 and 3 in the ring (Joule, John A.; Mills, Keith, eds. (2010). Heterocyclic Chemistry (5th ed.). Oxford: Wiley). The other diazines are pyrazine (nitrogen atoms at the 1 and 4 positions) and pyridazine (nitrogen atoms at the 1 and 2 positions).

The pyrimidine ring system has wide occurrence in nature (Lagoja, Irene M. (2005). “Pyrimidine as Constituent of Natural Biologically Active Compounds” (PDF). Chemistry and Biodiversity. 2 (1): 1-50) as substituted and ring fused compounds and derivatives, including the nucleotides cytosine, thymine and uracil, thiamine (vitamin B1) and alloxan. It is also found in many synthetic compounds such as barbiturates and the HIV drug, zidovudine. Exemplary pyrimidine compounds contemplated for inclusion and use in the compounds and methods of the instant disclosure include, without limitation:

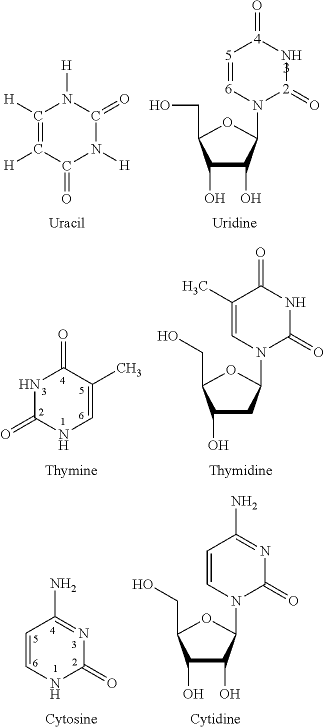

Additional pyrimidine-containing compounds, as well as analogs and/or derivatives thereof are also contemplated as pyrimidine compounds for purpose of certain aspects of the instant disclosure. Exemplary such pyrimidine-containing compounds include nucleoside analog agents, chemotherapies such as Tegafur/uracil (an oral agent which combines uracil with the 5-fluorouracil (5-FU) prodrug tegafur in a 4:1 molar ratio; see, e.g., U.S. Pat. No. 6,602,870), among others. In certain embodiments, compositions and methods that include or employ pharmaceutically acceptable salts of pyrimidine compounds are also expressly contemplated.

### Antimicrobial Agents

In certain aspects, the instant disclosure contemplates employment of pyrimidine compound(s) to potentiate the antimicrobial activity of antimicrobial agents, particularly bactericidal antimicrobial agents, such as antibiotics and antimicrobial peptides.

### Antibiotics

In certain aspects, the antibiotics of the instant disclosure include β-lactam antibiotics, quinolone antibiotics, aminoglycoside antibiotics, and/or carbapenem antibiotics (e.g., imipenem). In some aspects, the antibiotics of the instant disclosure include β-lactams, carbapenems (e.g., imipenem), aminoglycosides, fluoroquinolones, related quinolones and naphthyridines, chloramphenicol, macrolides, ketolides, azalides, Synercid®, tetracyclines, glycopeptides, novobiocin, oxazolidinones, cephalosporins, ceftazidime, ciprofloxacin, gentamicin, meropenem and the like, or a combination thereof. In certain embodiments, an exemplary antibiotic of a composition and/or method of the instant disclosure and/or that is potentiated via the compositions and/or methods of the instant disclosure is an aminoglycoside antibiotic (e.g., gentamicin, streptomycin, kanamycin), a β-lactam antibiotic (e.g., penicillins and cephalosporins), a vancomycin antibiotic, a bacitracin antibiotic, a macrolide antibiotic (e.g., erythromycins), a lincosamide antibiotic (e.g., clindomycin), a chloramphenicol antibiotic, a tetracycline antibiotic, an amphotericin antibiotic, a cefazolin antibiotic, a clindamycin antibiotic, a mupirocin antibiotic, a sulfonamide antibiotic, a trimethoprim antibiotic, a rifampicin antibiotic, a metronidazole antibiotic, a quinolone antibiotic, a novobiocin antibiotic, a polymixin antibiotic, a gramicidin antibiotic, alone or in combination, or any salts or variants thereof.

Antibiotics that Induce Resistance and/or Tolerance in Bacteria

Certain aspects of the present disclosure relate to compositions and methods that either include antibiotics to which bacteria are at risk of developing resistance and/or tolerance, and/or that potentiate the antibacterial effects of antibiotics to which bacteria develop resistance or tolerance and/or are at risk of developing resistance or tolerance. Exemplary antibiotics that induce resistance and/or tolerance in bacteria include those listed above and elsewhere herein, as well as those otherwise known in the art.

### β-Lactam Antibiotics

Certain aspects of the instant disclosure employ β-lactam antibiotics. β-lactam antibiotics are a class of broad-spectrum antibiotic that consists of antibiotic agents that contain a beta-lactam ring in their molecular structures. Exemplary β-lactam antibiotics include the following:
Penicillin derivatives (Penams), for which an exemplary dosage is a standard adult dosage between 0.2-1.0 g in a 6-24 hour interval: Specific examples include Benzathine penicillin (benzathine & benzylpenicillin), Benzylpenicillin (penicillin G), Phenoxymethylpenicillin (penicillin V), Procaine penicillin (procaine & benzylpenicillin), Pheneticillin, Cloxacillin, Dicloxacillin, Flucloxacillin, Methicillin, Nafcillin, Oxacillin, Temocillin, Amoxicillin, Ampicillin, Mecillinam, Carbenicillin, Ticarcillin, Azlocillin, Mezlocillin, and Piperacillin, with exemplary structures as shown below.

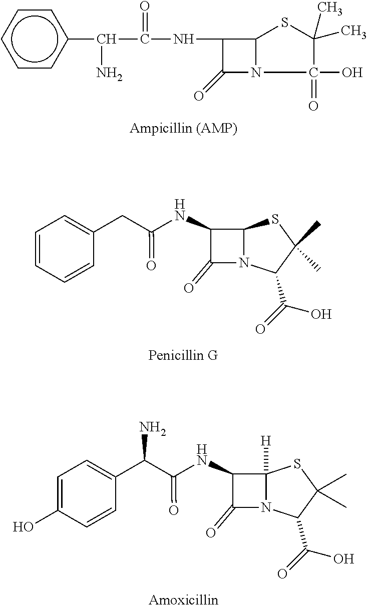

Cephalosporin (Cephems), for which an exemplary dosage is a standard adult dosage between 0.2-1.0 g in a 6-24 hour interval: Examples include Cefazolin, Cephalexin, Cephalosporin C, Cephalothin, Cefaclor, Cefamandole, Cefuroxime, Cefotetan, Cefoxitin, Cefixime, Cefotaxime, Cefpodoxime, Ceftazidime, Ceftriaxone, Cefepime, Cefpirome, and Ceftaroline. Exemplary structures for such compounds include the following.

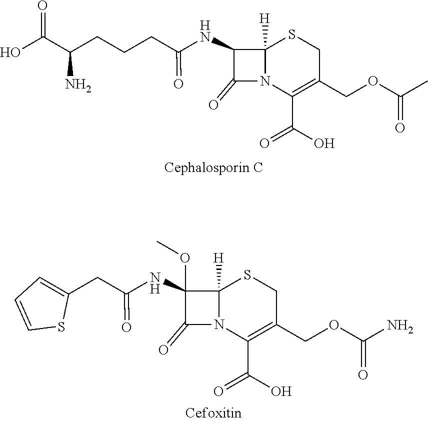 [SMILES: CC(=O)OCC1=C(C(=O)O)N2C(=O)[C@@H](NC(=O)CCC[C@@H](N)C(=O)O)[C@H]2SC1 ; CO[C@@]1(NC(=O)Cc2cccs2)C(=O)N2C(C(=O)O)=C(COC(N)=O)CS[C@@H]21]

Monobactams, for which an exemplary dosage is a standard adult dosage between 0.5-2.0 g in a 6-12 hour interval: Examples include Aztreonam, Tigemonam, Nocardicin A, and Tabtoxinine β-lactam (which does not inhibit penicillin-binding proteins). An exemplary structure for such compounds follows.

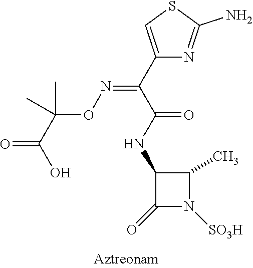 [SMILES: C[C@H]1[C@H](NC(=O)/C(=N\OC(C)(C)C(=O)O)c2csc(N)n2)C(=O)N1S(=O)(=O)O]

Carbapenems and Penems, for which an exemplary dosage is a standard dosage between 0.5-2.0 g in a 8 hour interval: Examples include Biapenem, Doripenem, Ertapenem, Faropenem, Imipenem, Meropenem, Panipenem, Razupenem, Tebipenem, and Thienamycin. An exemplary structure for such compounds follows.

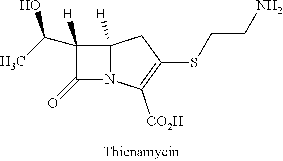 [SMILES: C[C@@H](O)[C@H]1C(=O)N2C(C(=O)O)=C(SCCN)C[C@H]12]

Most β-lactam antibiotics are believed to work by inhibiting cell wall biosynthesis in the bacterial organism. β-lactam antibiotics are the most widely used group of antibiotics. Until 2003, when measured by sales, more than half of all commercially available antibiotics in use were β-lactam compounds (Elander, R. P. Applied Microbiology and Biotechnology. 61: 385-392). β-lactam antibiotics are indicated for the prevention and treatment of bacterial infections caused by susceptible organisms. At first, β-lactam antibiotics were mainly active only against Gram-positive bacteria, yet the recent development of broad-spectrum β-lactam antibiotics active against various Gram-negative organisms has increased their usefulness.

### Quinolones

A quinolone antibiotic is a member of a large group of broad-spectrum bactericides that share a bicyclic core structure related to the compound 4-quinolone (Andriole, V T The Quinolones. Academic Press, 1989):

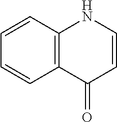 [SMILES: O=c1cc[nH]c2ccccc12]

Quinolone antibiotics are commonly used in human and veterinary medicine to treat bacterial infections, as well as in animal husbandry.

Nearly all quinolone antibiotics in use are fluoroquinolones, which contain a fluorine atom in their chemical structure and are effective against both Gram-negative and Gram-positive bacteria. One example is ciprofloxacin, one of the most widely used antibiotics worldwide (Andersson and MacGowan. Journal of Antimicrobial Chemotherapy. 51 (Suppl. S1): 1-11; Heeb et al. FEMS Microbiology Reviews. 35 (2): 247-274):

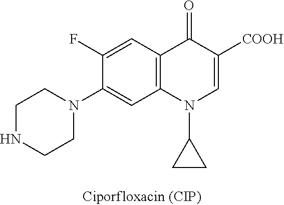 [SMILES: O=C(O)c1cn(C2CC2)c2cc(N3CCNCC3)c(F)cc2c1=O]

Exemplary approved dosages of Ciprofloxacin include 500 mg and 750 mg.

Fluoroquinolones are often used for genitourinary infections and are widely used in the treatment of hospital-acquired infections associated with urinary catheters. In community-acquired infections, they are recommended only when risk factors for multidrug resistance are present or after other antibiotic regimens have failed. However, for serious acute cases of pyelonephritis or bacterial prostatitis where the person may need to be hospitalised, fluoroquinolones are recommended as first-line therapy (Liu and Mulholland. American Journal of Medicine. 118 Suppl 7A (7): 14S-20S). Fluoroquinolones are also featured prominently in guidelines for the treatment of hospital-acquired pneumonia (Infectious Diseases Society of America (February 2005). “Guidelines for the management of adults with hospital-acquired, ventilator-associated, and healthcare-associated pneumonia”. American Journal of Respiratory and Critical Care Medicine. 171 (4): 388-416).

Additional exemplary quinolone antibiotics include garenoxacin (exemplary dosages: 400 mg, 600 mg), gatifloxacin (exemplary dosage: 400 mg), gemifloxacin (exemplary dosages: 320 mg, 640 mg), levofloxacin (exemplary dosages: 500 mg, 750 mg), moxifloxacin (exemplary dosages: 200 mg, 400 mg), fleroxacin, lomefloxacin, nadifloxacin, norfloxacin, ofloxacin, pefloxacin, rufloxacin, balofloxacin, grepafloxacin, pazufloxacin, sparfloxacin, temafloxacin, clinafloxacin, sitafloxacin, prulifloxacin, besifloxacin, delafloxacin, danofloxacin (for veterinary use), difloxacin (for veterinary use), enrofloxacin (for veterinary use), ibafloxacin (for veterinary use), marbofloxacin (for veterinary use), orbifloxacin (for veterinary use), and sarafloxacin (for veterinary use).

### Aminoglycosides

Aminoglycoside is a medicinal and bacteriologic category of traditional Gram-negative antibacterial medications that inhibit protein synthesis and contain as a portion of the molecule an amino-modified glycoside (sugar; see www.merriam-webster.com/medical/aminoglycoside: “any of a group of antibiotics (as streptomycin and neomycin) that inhibit bacterial protein synthesis and are active especially against gram-negative bacteria”; Mingeot-Leclercq et al. Antimicrob. Agents Chemother. 43 (4): 727-37). The term can also refer more generally to any organic molecule that contains amino sugar substructures. Aminoglycoside antibiotics display bactericidal activity against Gram-negative aerobes and some anaerobic bacilli where resistance has not yet arisen but generally not against Gram-positive and anaerobic Gram-negative bacteria (M E Levison, MD, 2012, Aminoglycosides, The Merck Manual).

In certain embodiments, the term “aminoglycoside antibiotic” refers to any naturally occurring drug, or semi-synthetic or synthetic derivative, comprising a highly-conserved aminocyclitol ring, which is a central scaffold that is linked to various amino-modified sugar moieties, that has antibiotic activity (as defined herein and/or, e.g., in U.S. Pat. No. 9,480,696, incorporated herein by reference). The ability to inhibit or reduce the growth of, or kill, one or more microorganisms is referred to herein as “antibiotic activity.” The aminocyclitol ring is comprised primarily of 2-deoxystreptamine (2-DOS) and has 1,3-diamino functionality and three or four hydroxyl groups that provide anchoring points for aminosugar moities. Aminoglycosides can be divided into 3 subclasses depending on the substitution pattern: 4-monosubsituted, or 4,5- or 4,6-disubstituted. Aminoglycosides in each subclass show close structural resemblance. Aminoglycosides have several mechanisms of antibiotic activity, including, but not limited to, inhibition of protein synthesis; interfering with proofreading processes during translation, and causing increased rate of error in synthesis with premature termination; inhibition of ribosomal translocation where the peptidyl-tRNA moves from the A-site to the P-site; disruption of bacterial cell membrane integrity; and/or binding to bacterial 30S ribosomal subunit.

Aminoglycosides have antibiotic activity against infections involving aerobic, gram-negative bacteria, such as, for example, Pseudomonas, Acinetobacter, and Enterobacter. In addition, some Mycobacteria, including the bacteria that cause tuberculosis, are susceptible to aminoglycosides. Aminoglycosides are also useful for empiric therapy for serious infections such as, for example, septicemia, intraabdominal infections, urinary tract infections, and respiratory tract infections, including nosocomial respiratory tract infections. Infections caused by gram-positive bacteria can also be treated with aminoglycosides, in some embodiments of the compositions and methods described herein. Non-limiting examples of aminoglycosides useful in the compositions and methods described herein include gentamicin, streptomycin, kanamycin A, tobramycin, neomycin B, neomycin C, framycetin, paromomycin, ribostamycin, amikacin, arbekacin, bekanamycin (kanamycin B), dibekacin, spectinomycin, hygromycin B, paromomycin sulfate, netilmicin, sisomicin, isepamicin, verdamicin, astromicin, neamine, ribostamycin, and paromomycinlividomycin, and derivatives thereof of each of these aminoglycoside antibiotics, including synthetic and semi-synthetic derivatives. Chemical structures of certain exemplary aminoglycoside antibiotics are provided and/or are known in the art. As understood by one of skill in the art, any compound or derivative having variations of the chemical structures of an aminoglycoside antibiotic, and having antibiotic activity, are suitable for use in embodiments of the methods and compositions provided herein (see, for example, “Aminoglycosides: Molecular Insights on the Recognition of RNA and Aminoglycoside Mimics,” Chittapragada M. et al., Perspectives in Medicinal Chemistry, 2009: 3 21-37, the contents of which are herein incorporated by reference in their entireties).

In some embodiments of the compositions and methods described herein, the aminoglycoside antibiotic is gentamicin. Gentamicin has the following structure:

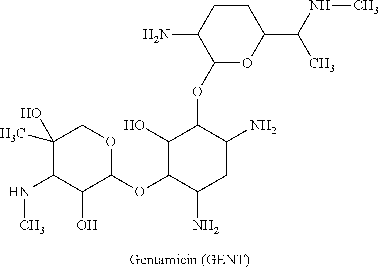

Gentamicin is an aminoglycoside antibiotic, used to treat many types of bacterial infections, particularly those caused by Gram-negative organisms. Gentamicin is a bactericidal antibiotic that works, in part, by binding the 30S subunit of the bacterial ribosome, interrupting protein synthesis. Like other aminoglycosides, when gentamicin is given orally, it is not systemically active. This is because it is not absorbed to any appreciable extent from the small intestine. Gentamicin can be administered intravenously, intramuscularly or topically to treat infections, in different embodiments of the methods and compositions described herein. It appears to be completely eliminated unchanged in the urine. Gentamicin is one of the few heat-stable antibiotics that remain active even after autoclaving, which makes it particularly useful in the preparation of some microbiological growth media. It can be used, for example, during orthopedic surgery when high temperatures are required for the setting of cements (e.g. hip replacements). Gentamicin is active against a wide range of human bacterial infections, mostly Gram-negative bacteria including, for example, Pseudomonas. Proteus. Serratia, and the Gram-positive Staphylococcus species. Gentamicin is also useful against Yersinia pestis, its relatives, and Francisella tularensis (the organism responsible for Tularemia seen often in hunters and/or trappers).

In some embodiments of the compositions and methods described herein, the aminoglycoside antibiotic is tobramycin. Tobramycin is an aminoglycoside antibiotic with activity against Gram-positive and Gram-negative bacteria. Tobramycin acts by inhibiting synthesis of protein in bacterial cells. Tobramycin has been shown to be active against most strains of the following organisms both in vitro and in clinical infections: aerobic and facultative Gram-positive microorganisms, including, for example, Staphylococcus aureus; aerobic and facultative Gram-negative microorganisms, including, for example, Citrobacter sp., Enterobacter sp. (e.g., E. aerogenes, E. cloacae), Escherichia coli, Klebsiella sp., Morganella morganii, Pseudomonas aeruginosa, Proteus mirabilis. Proteus vulgaris, Providencia sp., and Serratia sp.

In some embodiments of the compositions and methods described herein, the aminoglycoside antibiotic is netilmicin. Netilmicin is not absorbed from the gut and can therefore be given by injection or infusion. It can be used in the treatment of serious infections particularly those resistant to gentamicin.

In some embodiments of the compositions and methods described herein, the aminoglycoside antibiotic is Sisomicin (BACTOCEAZE or ENSAMYCIN), isolated from fermentation broth of a species of the genus Micromonospora. It is a newer broad-spectrum aminoglycoside, most structurally related to gentamicin. Sisomicin is a predictably active aminoglycoside against gram-positive bacteria. Like other aminoglycosides, Sisomicin is bactericidal for sensitive clinical isolates. The Minimum Bactericidal Concentrations (MBC) for Sisomicin have been found to be equivalent or very close to the Minimum Inhibitory Concentrations (MIC). Most clinical isolates of Pseudomonas aeruginosa remain susceptible to sisomicin.

In some embodiments of the compositions and methods described herein, the aminoglycoside antibiotic is arbekacin, a semisynthetic aminoglycoside antibiotic. It is primarily used for the treatment of infections caused by multi-resistant bacteria including methicillin-resistant Staphylococcus aureus (MRSA). Arbekacin was originally synthesized from dibekacin in 1973. It has been registered and marketed in Japan since 1990 under the trade name HABEKACIN. Generic versions of the drug are also available under such trade names as DECONTASIN and BLUBATOSINE. Arbekacin is used for the short term treatment of multi-resistant bacterial infections, such as, for example, methicillin-resistant Staphylococcus aureus (MRSA), as well as enteric bacteria and other eubacteria. Arbekacin works by binding to the bacterial 30S ribosomal subunit, causing misreading of t-RNA which consequently, leaves the bacterium unable to synthesize proteins vital to its growth. Specifically, arbekacin binds to four nucleotides of 16S rRNA and a single amino acid of protein S12. This interferes with decoding site in the vicinity of nucleotide 1400 in 16S rRNA of 30S subunit. This region interacts with the wobble base in the anticodon of tRNA. This leads to misreading of mRNA so incorrect amino acids are inserted into the polypeptide leading to nonfunctional or toxic peptides and the breakup of polysomes into nonfunctional monosomes. Arbekacin, like other aminoglycosides, is not well absorbed from the gastrointestinal tract. Its absorption is markedly improved by parenteral administration. Normal duration of IM or IV arbekacin aminoglycoside antibiotic therapy is 7-10 days, although a longer duration may be necessary in some cases.

In some embodiments of the compositions and methods described herein, the aminoglycoside antibiotic is amikacin, an aminoglycoside antibiotic used to treat different types of bacterial infections. Amikacin works by binding to the bacterial 30S ribosomal subunit, causing misreading of mRNA and leaving the bacterium unable to synthesize proteins vital to its growth. Amikacin is often used for Gram negative bacteria such as Pseudomonas aeruginosa, Acinetobacter, and Enterobacter, including, for example, treating severe, hospital-acquired infections with multidrug resistant Pseudomonas aeruginosa, Acinetobacter (e.g., Acinetobacter baumannii, Acinetobacter calcoaceticus-baumanii complex, Acinetobacter lwoffii, Acinetobacter haemolyticus, etc.), and Enterobacter, Serratia marcescens and Providencia stuartii are also included in the spectrum. Amikacin can also be used to treat non tubercular mycobacterial infections and tuberculosis, when first line drugs fail to control the infection. Amikacin can be combined, in some embodiments, with a beta-lactam antibiotic for empiric therapy for people with neutropenia and fever. In some embodiments of the methods and compositions described herein, amikacin can be combined or administered with fosfomycin (also known as phosphomycin, phosphonomycin and the trade names MONUROL and MONURIL), for treatment of certain infections, such as for example, urinary tract infection.

In some embodiments of the compositions and methods described herein, the aminoglycoside antibiotic is neomycin. Neomycin is overwhelmingly used as a topical preparation, such as NEOSPORIN. It can also, in some embodiments, be administered orally. Neomycin is not absorbed from the gastrointestinal tract and has been used as a preventive measure for hepatic encephalopathy and hypercholesterolemia. By killing bacteria in the intestinal tract, it keeps ammonia levels low and prevents hepatic encephalopathy, especially prior to GI surgery. It has also been used to treat small intestinal bacterial overgrowth. Similar to other aminoglycosides, neomycin has excellent activity against Gram-negative bacteria, and has partial activity against Gram-positive bacteria. It is not given intravenously, as neomycin is extremely nephrotoxic (causes kidney damage), especially compared to other aminoglycosides. The exception is when neomycin is included, in very small quantities, as a preservative in some vaccines—typically 0.025 mg per dose.

In some embodiments of the compositions and methods described herein, the aminoglycoside antibiotic is framycetin. Framycetin is commonly sold as SOFRAMYCIN and SOFRA-TULLE. It also exists in veterinary products, as Framycetin sulf. Like neomycin, framycetin has poor systemic absorption. It can be used in topical preparations for infections of the skin, nose, ears, and eyes, for example, in combination with other antibacterial drugs and corticosteroids, in some embodiments. It can also be used for gastrointestinal infections, in some embodiments.

In some embodiments of the compositions and methods described herein, the aminoglycoside antibiotic is paromomycin (brand name HUMATIN), first isolated from Streptomyces krestomuceticus in the 1950s. It is also called monomycin and aminosidine. Paromomycin is an antibiotic designed to fight intestinal infections such as cryptosporidiosis, amoebiasis, and leishmaniasis. The route of administration of paromomycin can be intramuscular injection and capsule, in some embodiments.

In some embodiments of the compositions and methods described herein, the aminoglycoside antibiotic is streptomycin. Streptomycin is a protein synthesis inhibitor, and binds to the small 16S rRNA of the 30S subunit of the bacterial ribosome, interfering with the binding of formyl-methionyl-tRNA to the 30S subunit. This leads to codon misreading, eventual inhibition of protein synthesis and ultimately death of microbial cells through mechanisms that are still not understood. Streptomycin is an antibiotic that inhibits both Gram-positive and Gram-negative bacteria, and is therefore a useful broad-spectrum antibiotic. Exemplary chronic infections that can be treated using the compositions and methods described herein comprising streptomycin as the aminoglycoside antibiotic include, but are not limited to, infective endocarditis caused by enterococcus when the organism is not sensitive to Gentamicin, tuberculosis in combination with other anti-TB drugs, and plague (Yersinia pestis). Streptomycin is also useful in the compositions and methods described herein for use in veterinary medicine applications, such as in treatments against gram negative bacteria in large animals (e.g., horses, cattle, sheep etc.). Streptomycin can also be used, in some embodiments of the compositions and methods described herein, as a pesticide, to combat the growth of bacteria, fungi, and algae. Streptomycin inhibits/reduces bacterial and fungal diseases of certain fruit, vegetables, seed, and ornamental crops, and inhibits/reduces algae in ornamental ponds and aquaria, as well as inhibits/reduces fireblight on apple and pear trees.

In some embodiments of the compositions and methods described herein, the aminoglycoside antibiotic is kanamycin or kanamycin A. Kanamycin interacts with the 30S subunit of prokaryotic ribosomes. Kanamycin A induces substantial amounts of mistranslation and indirectly inhibits translocation during protein synthesis. Kanamycin A is available in oral, intravenous, and intramuscular forms; can be administered via aerosol formulation or irrigation, and can be used to treat a wide variety of infections; and is used in form of the sulfate.

### Rifampicin

Rifampicin, also known as rifampin, is an antibiotic used to treat several types of bacterial infections, including tuberculosis. Mycobacterium avium complex, leprosy, and Legionnaires' disease (“Rifampin”. The American Society of Health-System Pharmacists. Archived from the original on 2015 Sep. 7). It is almost always used together with other antibiotics, except when given to prevent Haemophilus influenzae type b and meningococcal disease in people who have been exposed to those bacteria (ibid.). Before treating a person for a long period of time, measurements of liver enzymes and blood counts are recommended (ibid.). Rifampicin may be given either by mouth or intravenously.

Rifampicin is of the rifamycin group of antibiotics (ibid.; rifamycins are synthesized either naturally by the bacterium Amycolatopsis rifamycinica or artificially, and additional examples include Rifabutin, Rifapentine, Rifaximin, Rifalazil, Rifamycin B, Rifamycin SV and Rifamycin S, as well as derivatives thereof). Rifampicin is believed to act by stopping the production of RNA by bacteria (ibid.). The structure of rifampicin is shown below:

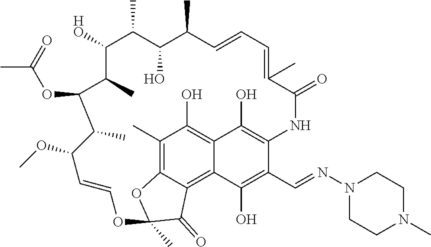 [SMILES: CO[C@H]1/C=C/O[C@@]2(C)Oc3c(C)c(O)c4c(O)c(c(/C=N/N5CCN(C)CC5)c(O)c4c3C2=O)NC(=O)/C(C)=C/C=C/[C@H](C)[C@H](O)[C@@H](C)[C@@H](O)[C@@H](C)[C@H](OC(C)=O)[C@@H]1C]

Rifampicin is commonly used for the treatment of tuberculosis in combination with other antibiotics, such as pyrazinamide, isoniazid, and ethambutol (World Health Organization (2010). Treatment of tuberculosis: guidelines). For the treatment of tuberculosis, it is administered daily for at least 6 months (Long, James W. (1991). Essential Guide to Prescription Drugs 1992. New York: HarperCollins Publishers. pp. 925-929). Combination therapy is used to prevent the development of resistance and to shorten the length of treatment (Erlich et al. Molecular Biology of Rifamycin. New York, N.Y.: MSS Information Corporation. pp. 44-45, 66-75, 124-130). Resistance of Mycobacterium tuberculosis to rifampicin develops quickly when it is used without another antibiotic, with laboratory estimates of resistance rates from 10⁻⁷ to 10⁻¹⁰ per tuberculosis bacterium per generation (Goldstein B P. The Journal of Antibiotics. 67: 625-30: David H L. Applied Microbiology. 20: 810-4).

Rifampicin can be used alone in patients with latent tuberculosis infections to prevent or prolong the development of active disease because only small numbers of bacteria are present. Rifampicin may also be useful in the treatment of methicillin-resistant Staphylococcus aureus (MRSA) in combination with other antibiotics, including in difficult-to-treat infections such as osteomyelitis and prosthetic joint infections (Perlroth et al Archives of Internal Medicine. 168: 805-19). As of 2012, if rifampicin combination therapy was useful for pyogenic vertebral osteomyelitis was unclear (Pola et al. European Review for Medical and Pharmacological Sciences. 16 Suppl 2: 35-49). A meta-analysis concluded that adding adjunctive rifampicin to a β-lactam or vancomycin may improve outcomes in Staphylococcus aureus bacteremia (Russell et al. Journal of Medical Microbiology. 63: 841-8). However, a more recent trial found no benefit from adjunctive rifampicin (Thwaites et al. Lancet. 391: 668-67).

Rifampicin is also used as preventive treatment against Neisseria meningitidis (meningococcal) infections, and is recommended as an alternative treatment for infections by the tick-borne pathogens Borrelia burgdorferi and Anaplasma phagocytophilum when treatment with doxycycline is contraindicated, such as in pregnant women or in patients with a history of allergy to tetracycline antibiotics (Wormser et al. Clinical Infectious Diseases. 43: 1089-134; Thomas et al. Expert Review of Anti-Infective Therapy. 7: 709-22). Rifampicin is also sometimes used to treat infections by Listeria species, Neisseria gonorrhoeae, Haemophilus influenzae, and Legionella pneumophila. For these nonstandard indications, antimicrobial susceptibility testing should be done (if possible) before starting rifampicin therapy.

Rifampicin has been used with amphotericin B in largely unsuccessful attempts to treat primary amoebic meningoencephalitis caused by Naegleria fowleri.

Rifampicin can be used as monotherapy for a few days as prophylaxis against meningitis, but resistance develops quickly during long-term treatment of active infections, so the drug is always used against active infections in combination with other antibiotics (“Rifampicin”. Archived from the original on Oct. 2, 2014).

Rifampicin has some effectiveness against vaccinia virus (Charity et al. Virology. 359: 227-32; Sodeik et al. Journal of Virology. 68: 1103-14).

Rifampicin capsules for oral administration contain 150 mg or 300 mg rifampicin per capsule. Rifampicin for intravenous injection contains 600 mg, together with sodium formaldehyde sulfoxylate 10 mg and sodium hydroxide to adjust pH.

### Nitrofurantoin

Nitrofurantoin is an antibiotic used to treat bladder infections, and is taken by mouth (“Nitrofurantoin”. The American Society of Health-System Pharmacists. Archived from the original on 2015 Jul. 7). It is available as a generic medication (ibid.). Current uses include the treatment of uncomplicated urinary tract infections (UTIs) and prophylaxis against UTIs in people prone to recurrent UTIs (“Macrobid Drug Label”. FDA. Archived from the original on 21 Apr. 2014). The chemical structure of nitrofurantoin is:

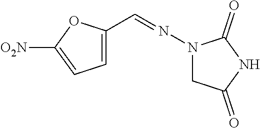 [SMILES: O=C1CN(N=Cc2ccc([N+](=O)[O-])o2)C(=O)N1]

Increasing bacterial antibiotic resistance to other commonly used agents, such as fluoroquinolones and trimethoprim/sulfamethoxazole, has led to increased interest in using nitrofurantoin (Garau J. Clin. Microbiol. Infect. 14 Suppl 1: 198-202; McKinnell et al. Mayo Clinic Proceedings. 86: 480-8). The efficacy of nitrofurantoin in treating UTIs combined with a low rate of bacterial resistance to this agent makes it one of the first-line agents for treating uncomplicated UTIs as recommended by the Infectious Diseases Society of America and the European Society for Microbiology and Infectious Diseases (Gupta et al. Clinical Infectious Diseases. 52: e103-e120).

Nitrofurantoin has been shown to have good activity against E. coli, Staphylococcus saprophyticus, coagulase negative staphylococci, Enterococcus faecalis, Staphylococcus aureus, Streptococcus agalactiae, Citrobacter species, Klebsiella species and Bacillus subtilis species (Gupta et al. JAMA: The Journal of the American Medical Association. 281: 736-8). Many or all strains of the following genera are resistant to nitrofurantoin: Enterobacter, Klebsiella, Proteus and Pseudomonas (ibid.).

There are currently two formulations of nitrofurantoin: (1) Macrocrystals—(Macrodantin, Furadantin)—25, 50, or 100 mg capsules—taken once every 6 hours; and (2) Monohydrate/macrocrystals—(Macrobid)—100 mg capsules—taken once every 12 hours or 2 times a day (“Drugs for bacterial infections”. Treatment Guidelines from the Medical Letter. 11: 65-74), which is 75% monohydrate and 25% macrocrystals (“Nitrofurantoin Capsules—FDA prescribing information, side effects and uses”. Drugs.com. Retrieved 28 Nov. 2017).

Other nitrofuran derivatives with use similar to Nitrofurantoin include Furazolidone and Nifurtoinol.

### Metronidazole

Metronidazole is an antibiotic and antiprotozoal medication (“Metronidazole”. The American Society of Health-System Pharmacists. Archived from the original on Sep. 6, 2015), which has the following chemical structure:

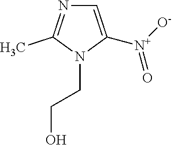

Metronidazole is used either alone or with other antibiotics to treat pelvic inflammatory disease, endocarditis, and bacterial vaginosis (ibid.). It is effective for dracunculiasis, giardiasis, trichomoniasis, and amebiasis (ibid.). It is an option for a first episode of mild-to-moderate Clostridium difficile colitis if vancomycin or fidaxomicin is unavailable (ibid.; McDonald et al. “Clinical Practice Guidelines for Clostridium difficile Infection in Adults and Children: 2017 Update by the Infectious Diseases Society of America (IDSA) and Society for Healthcare Epidemiology of America (SHEA)”. Clinical Infectious Diseases.). Metronidazole is available by mouth, as a cream, and by injection into a vein (“Metronidazole”).

Metronidazole is primarily used to treat: bacterial vaginosis, pelvic inflammatory disease (along with other antibacterials like ceftriaxone), pseudomembranous colitis, aspiration pneumonia, rosacca (topical), fungating wounds (topical), intra-abdominal infections, lung abscess, periodontitis, amoebiasis, oral infections, giardiasis, trichomoniasis, and infections caused by susceptible anaerobic organisms such as Bacteroides, Fusobacterium, Clostridium, Peptostreptococcus, and Prevotella species (Rossi. S. ed. (2013). Australian Medicines Handbook (2013 ed.). Adelaide: The Australian Medicines Handbook Unit Trust). It is also often used to eradicate Helicobacter pylori along with other drugs and to prevent infection in people recovering from surgery (ibid.).

Metronidazole is bitter and so the liquid suspension contains metronidazole benzoate.

Drugs of choice for the treatment of bacterial vaginosis include metronidazole and clindamycin. The treatment of choice for bacterial vaginosis in nonpregnant women include metronidazole oral twice daily for seven days, or metronidazole gel intravaginally once daily for five days, or clindamycin intravaginally at bedtime for seven days. For pregnant women, the treatment of choice is metronidazole oral three times a day for seven days.

The 5-nitroimidazole drugs (metronidazole and tinidazole) are the mainstay of treatment for infection with Trichomonas vaginalis. Treatment for both the infected patient and the patient's sexual partner is recommended, even if asymptomatic.

Oral metronidazole is a treatment option for giardiasis, however, the increasing incidence of nitroimidazole resistance is leading to the increased use of other compound classes (Leitsch, David. Current Tropical Medicine Reports. 2: 128).

Initial antibiotic therapy for less-severe Clostridium difficile colitis (pseudomembranous colitis) consists of metronidazole, vancomycin, or fidaxomicin by mouth (McDonald et al.). In 2017 the IDSA generally recommended vancomycin and fidaxomicin over metronidazole (ibid.). Vancomycin by mouth has been shown to be more effective in treating people with severe C. difficile colitis (Zar et al. Clinical Infectious Diseases. 45: 302-7).

Entamoeba histolytica invasive amebiasis is treated with metronidazole for eradication, in combination with diloxanide to prevent recurrence (Sherris medical microbiology. Ryan, Kenneth J. (Kenneth James), 1940-(Seventh ed.). New York).

Metronidazole is available as an oral powder (7.5 g, 15 g), an oral tablet (750 mg, 500 mg, 250 mg), an intravenous injectate (1 mL, 5 mg), an oral caplet (375 mg), a topical cream (0.75%, 1%), a vaginal gel (0.75%, 1.3%), a topical gel (0.75%, 1%), and a topical lotion (0.75%).

### Trimethoprim

Trimethoprim is an antibiotic used mainly in the treatment of bladder infections (“Trimethoprim”. The American Society of Health-System Pharmacists. Archived from the original on 2015 Sep. 24). Other uses include for middle ear infections and travelers' diarrhea (ibid.). Optionally together with sulfamethoxazole or dapsone, trimethoprim may be used for Pneumocystis pneumonia in people with HIV/AIDS (ibid.; Masur et al. Clinical Infectious Diseases. 58: 1308-11). It is taken by mouth, and is available as an oral solution (5 mL, 50 mg), or as an oral tablet (100 mg; “Trimethoprim”). It is believed to work by blocking folate metabolism via dihydrofolate reductase in some bacteria, which results in their death (ibid.).

The chemical structure of trimethoprim is:

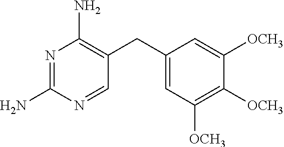 [SMILES: COc1cc(Cc2cnc(N)nc2N)cc(OC)c1OC]

Trimethoprim is primarily used in the treatment of urinary tract infections, although it may be used against any susceptible aerobic bacterial species (Rossi, S, ed. (2013). Australian Medicines Handbook (2013 ed.). Adelaide: The Australian Medicines Handbook Unit Trust). It may also be used to treat and prevent Pneumocystis jirovecii pneumonia (ibid.). It is generally not recommended for the treatment of anaerobic infections such as Clostridium difficile colitis (the leading cause of antibiotic-induced diarrhea; ibid.). Trimethoprim has also been used in trials to treat retinitis (Pradhan et al. Cochrane Database Syst Rev (5): CD002218).

Exemplary trimethoprim-susceptible bacterial species include Escherichia coli, Proteus mirabilis, Klebsiella pneuimoniae, Enterobacter species, coagulase-negative Staphylococcus species, including S. saprophyticus, Streptococcus pneumoniae, and Haemophilus influenza.

Without wishing to be bound by theory, trimethoprim has been shown to bind to dihydrofolate reductase and inhibit the reduction of dihydrofolic acid (DHF) to tetrahydrofolic acid (THF; Brogden et al. Drugs. 23: 405-30). THF is an essential precursor in the thymidine synthesis pathway and interference with this pathway inhibits bacterial DNA synthesis (ibid.). Trimethoprim's affinity for bacterial dihydrofolate reductase is several thousand times greater than its affinity for human dihydrofolate reductase (ibid.). Sulfamethoxazole inhibits dihydropteroate synthase, an enzyme involved further upstream in the same pathway (ibid.). Trimethoprim and sulfamethoxazole are commonly used in combination due to possible synergistic effects, and potential for reduced development of resistance (ibid.).

Related DHFR inhibitors, which are also contemplated for inclusion in the compositions and methods of the instant disclosure, include non-Trimethoprim 2,4-Diaminopyrimidine compounds, such as Brodimoprim, Tetroxoprim, and Iclaprim.

### Sulfamethoxazole

Sulfamethoxazole is used for bacterial infections such as urinary tract infections, bronchitis, and prostatitis and is effective against both gram negative and positive bacteria such as Listeria monocytogenes and E. coli (“Sulfamethoxazole”. DrugBank. Retrieved 5 Nov. 2015). It is a sulfonamide and bacteriostatic. Sulfamethoxazole resembles a component of folic acid and has the following structure:

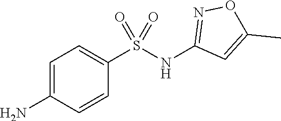 [SMILES: Cc1cc(NS(=O)(=O)c2ccc(N)cc2)no1]

Sulfamethoxazole prevents folic acid synthesis in bacteria that must synthesize their own folic acid. Mammalian cells, and some bacteria, do not synthesize but require preformed folic acid (vitamin B9), they are therefore insensitive to sulfamethoxazole (Brunton et al. (2011). Goodman and Gilman's The pharmacological Basis of Therapeutics. The McGraw-Hill Companies, Inc. pp. 1463-1469).

Sulfamethoxazole, a sulfanilamide, is a structural analog of para-aminobenzoic acid (PABA). They compete with PABA to bind to dihydropteroate synthetase and inhibit conversion of PABA and dihydropteroate diphosphate to dihydrofolic acid, or dihydrofolate. Inhibiting the production of dihydrofolate intermediate interferes with the normal bacterial synthesis of folic acid (folate). Folate is an essential metabolite for bacterial growth and replication because it is used in DNA synthesis, primarily at thymidylate and purine biosynthesis, and amino acids synthesis, including serine, glycine and methionine (“Sulfonamides and Sulfonamide Combinations: Antibacterial Agents: Merck Veterinary Manual”. www.merckvetmanual.com). Hence, blockage of folate production inhibits the folate-dependent metabolic processes for bacterial growth. Since it inhibits bacterial growth, sulfamethoxazole is considered a bacteriostatic antibiotic (“Sulfamethoxazole”. DrugBank. Retrieved 5 Nov. 2015).

It is believed that sulfonamides are selective against bacteria because they interfere with the synthesis of folate, a process which does not occur in humans. Humans do not synthesize folate, and must acquire it through diet (“Sulfonamides—Infectious Diseases”. Merck Manuals Professional Edition. Retrieved 2015 Nov. 5).

Sulfamethoxazole distributes into most body tissues as well as into sputum, vaginal fluid, and middle ear fluid (“Bactrim USPI” (PDF). FDA; “Toxnet: Sulfamethoxazole”). It also crosses the placenta. About 70% of the drug is bound to plasma proteins. Its Tmax (or time to reach maximum drug concentration in plasma) occurs 1 to 4 hours after oral administration. The mean serum half-life of sulfamethoxazole is 10 hours (“Bactrim USPI” (PDF). FDA). However, the half-life of the drug noticeably increases in people with creatinine clearance rates equal to or less than 30 mL/minute. A half-life of 22-50 hours has been reported for people with creatinine clearances of less than 10 mL/minute (“Toxnet: Sulfamethoxazole”).

Sulfamethoxazole is metabolized in the human liver to at least 5 metabolites. These metabolites are the N4-acetyl-, N4-hydroxy-, 5-methylhydroxy-, N4-acetyl-5-methylhydroxy-sulfamethoxazole metabolites, and an N-glucuronide conjugate. The CYP2C9 enzyme is responsible for the formation of the N4-hydroxy metabolite. In vitro studies have indicated that sulfamethoxazole is not a substrate of the P-glycoprotein transporter (“Bactrim USPI” (PDF). FDA).

Sulfamethoxazole is primarily renally excreted via glomerular filtration and tubular secretion (“Bactrim USPI” (PDF). FDA). About 20% of the sulfamethoxazole in urine is the unchanged drug, about 15-20% is the N-glucuronide conjugate, and about 50-70% is the acetylated metabolite (“Toxnet: Sulfamethoxazole”). Sulfamethoxazole is also excreted in human milk (“Bactrim USPI” (PDF). FDA).

Sulfamethoxazole is primarily available as a Sulfamethoxazole, Trimethoprim Oral Tablet (400-80 mg, 800-160 mg), as a Septra/Sulfamethoxazole, Trimethoprim/Sulfatrim/Sulfatrim Pediatric/Sultrex Pediatric Oral Suspension (5 mL, 200-40 mg), or as a Sulfamethoxazole, Trimethoprim intravenous injectate solution (1 mL, 80-16 mg).

Sulfamethoxazole is an exemplary sulfonamide; Other sulfonamides—which are also contemplated for inclusion in the compositions and methods of the instant disclosure—include Sulfaisodimidine, Sulfamethizole, Sulfadimidine. Sulfapyridine. Sulfafurazole, Sulfanilamide (Prontosil), Sulfathiazole, Sulfathiourea, Sulfadiazine, Sulfamoxole, Sulfadimethoxine, Sulfadoxine, Sulfalene, Sulfametomidine, Sulfametoxydiazine, Sulfamethoxypyridazine, Sulfaperin, Sulfamerazine, Sulfaphenazole, Sulfamazone, Sulfacetamide, Sulfadicramide, Sulfametrole, and Sulfanitran.

Combination drugs that incorporate sulfonamides include Trimethoprim/sulfamethoxazole and Ormetoprim/sulfadimethoxine. Meanwhile, non-sulfonamide DHPS inhibitors include Dapsone.

The above-recited compounds, as well as related compounds (e.g., analogs) and derivatives thereof, are contemplated for inclusion in the compounds and methods of the instant disclosure.

### Antimicrobial Peptides

Antimicrobial peptides (AMPs), also called host defense peptides (HDPs) are part of the innate immune response found among all classes of life. Fundamental differences exist between prokaryotic and eukaryotic cells that may represent targets for antimicrobial peptides. These peptides are potent, broad spectrum antibiotics which demonstrate potential as therapeutic agents. Antimicrobial peptides have been demonstrated to kill Gram negative and Gram positive bacteria, enveloped viruses, fungi and even transformed or cancerous cells (Reddy et al. International Journal of Antimicrobial Agents. 24 (6): 536-47). Unlike the majority of conventional antibiotics, it appears that antimicrobial peptides frequently destabilize biological membranes, can form transmembrane channels, and may also have the ability to enhance immunity by functioning as immunomodulators.

Antimicrobial peptides are a unique and diverse group of molecules, which are divided into subgroups on the basis of their amino acid composition and structure (Yeaman and Yount. Pharmacological Reviews. 55 (1): 27-55). Antimicrobial peptides are generally between 12 and 50 amino acids. These peptides include two or more positively charged residues provided by arginine, lysine or, in acidic environments, histidine, and a large proportion (generally >50%) of hydrophobic residues (Papagianni M. Biotechnology Advances. 21 (6): 465-99; Sitaram and Nagaraj. Current Pharmaceutical Design. 8 (9): 727-42; Dürr et al. Biochimica et Biophysica Acta. 1758 (9): 1408-25). The secondary structures of these molecules follow 4 themes, including i) α-helical, ii) β-stranded due to the presence of 2 or more disulfide bonds, iii) β-hairpin or loop due to the presence of a single disulfide bond and/or cyclization of the peptide chain, and iv) extended (Dhople et al. Biochimica et Biophysica Acta. 1758 (9): 1499-512). [6] Many of these peptides are unstructured in free solution, and fold into their final configuration upon partitioning into biological membranes. It contains hydrophilic amino acid residues aligned along one side and hydrophobic amino acid residues aligned along the opposite side of a helical molecule (Yeaman and Yount). This amphipathicity of the antimicrobial peptides allows them to partition into the membrane lipid bilayer. The ability to associate with membranes is a definitive feature of antimicrobial peptides (Hancock and Rozek. FEMS Microbiology Letters. 206 (2): 143-9; Varkey et al. Peptides. 27 (11): 2614-23) although membrane permeabilization is not necessary. These peptides have a variety of antimicrobial activities ranging from membrane permeabilization to action on a range of cytoplasmic targets.

Antimicrobial peptides have been used as therapeutic agents. Therapeutic antimicrobial peptide use is generally limited to intravenous administration or topical applications, due to their short half-lives. The following antimicrobial peptides are among those currently in clinical use (Järvå et al. Science Advances. 4 (7): eaat0979): Bacitracin (in clinical use for pneumonia, topical); Boceprevir (in clinical use for Hepatitis C (oral, cyclic peptide)); Dalbavancin (in clinical use for bacterial infections, IV); Daptomycin (in clinical use for bacterial infections, IV); Enfuvirtide (in clinical use for HIV, subcutaneous injection); Oritavancin (in clinical use for bacterial infections, IV); Teicoplanin (in clinical use for bacterial infections, IV); Telaprevir (in clinical use for Hepatitis C, oral cyclic peptide); Telavancin (in clinical use for bacterial infection, IV); Vancomycin (in clinical use for bacterial infection, IV); and Guavanin 2 (in clinical use for bacterial infection against Gram-positive and Gram-negative also).

### β-Lactamase Inhibitors

Certain aspects of the instant disclosure can include and/or employ β-lactamase inhibitors. In certain embodiments, β-lactamase inhibitors can be compounded together with antibiotics to potentiate antibiotic efficacy. β-lactamases are a family of enzymes involved in bacterial resistance to β-lactam antibiotics. Without wishing to be bound by theory, β-lactamases tend to act by breaking the beta-lactam ring that is believed to be necessary for the antimicrobial activity of penicillin-like antibiotics. With the goal of enhancing the activity of penicillin-like compounds in the presence of β-lactamases, β-lactamase inhibitors have been developed (Essack S Y. Pharmaceutical Research. 18: 1391-9). Although β-lactamase inhibitors possess little antibiotic activity of their own (“Beta-Lactamase Inhibitors”. Department of Nursing of the Fort Hays State University College of Health and Life Sciences. October 2000), they prevent bacterial degradation of beta-lactam antibiotics and can extend the range of bacteria such drugs are effective against.

β-lactamase inhibitors are generally co-formulated with a β-lactam antibiotic possessing a similar serum half-life. This is done to minimize resistance development that might occur as a result of varying exposure to one or the other drug. The main classes of β-lactam antibiotics used to treat gram-negative bacterial infections include penicillins, 3rd generation cephalosporins, and carbapenems. β-lactamase inhibitors expand the useful spectrum of these β-lactam antibiotics by inhibiting the β-lactamase enzymes produced by bacteria to deactivate them (Watson et al. Clinical Pharmacokinetics. 15: 133-64).

Exemplary β-lactamase inhibitors possessing a β-lactam core include:
Tebipenem, which was the first carbapenem to be administered orally in the form of Tebipenem-Pivoxil.
Boron based transition state inhibitors (“BATSIs”), which constitute a very potent group of beta-lactamase inhibitors. An exemplary BATSI is Ec19.
Clavulanic acid or clavulanate, which is often combined with amoxicillin (Augmentin) or ticarcillin (Timentin). Clavulanic Acid has the following exemplary structure:

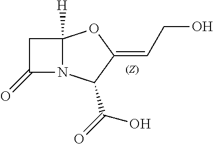 [SMILES: O=C(O)[C@H]1/C(=C/CO)O[C@@H]2CC(=O)N21]

Sulbactam, which is often combined with ampicillin (Unasyn) or Cefoperazone (Sulperazon). Sulbactam has the following structure:

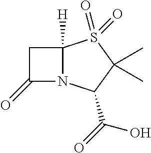

Tazobactam, which is often combined with piperacillin (Zosyn) (Tazocin). Tazobactam has the following structure:

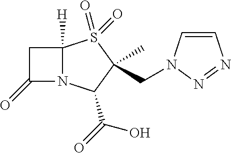 [SMILES: C[C@]1(Cn2ccnn2)[C@H](C(=O)O)N2C(=O)C[C@H]2S1(=O)=O]

Exemplary non-β-lactam β-lactamase inhibitors include:
Avibactam, which has been FDA approved in combination with ceftazidime (Avycaz), and is currently undergoing clinical trials for combination with ceftaroline. Avibactam has the following structure:

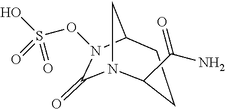 [SMILES: NC(=O)C1CCC2CN1C(=O)N2OS(=O)(=O)O]

Relebactam (previously known as MK-7655) is undergoing Phase III clinical trials as a treatment for pneumonia and bacterial infections. Relebactam has the following structure:

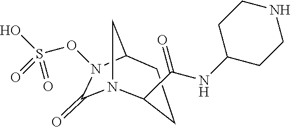 [SMILES: O=C(NC1CCNCC1)C1CCC2CN1C(=O)N2OS(=O)(=O)O]

Antibiotic Resistant and/or Antibiotic Tolerant Bacteria

In certain aspects, the present disclosure provides compositions and/or methods designed to inhibit the growth of and/or kill bacteria, particularly bacteria that have become or are at risk of becoming antibiotic tolerant and/or antibiotic resistant. Tolerance specifically refers to an inability of high concentrations of antibiotics—typically lethal concentrations that are above the growth-inhibitory threshold for a given strain—to kill bacteria. Tolerance levels can be influenced by genetic mutations or induced by environmental conditions. Bacteria can often develop antibiotic tolerance and/or resistance. Resistance can tend to arise via mutations that confer increased survival, which are selected for in natural selection, and which can arise quickly in bacteria because lifespans and production of new generations can be on a timescale of mere hours. Tolerant and/or resistant microbes are more difficult to treat, requiring alternative medications or higher doses of antimicrobials. These approaches may be more expensive, more toxic or both. Microbes resistant to multiple antimicrobials are called multidrug resistant (MDR). Those considered extensively drug resistant (XDR) or totally drug resistant (TDR) are sometimes called “superbugs”.

An exemplary list of Gram-positive bacteria that have been shown to possess antibiotic resistance or have been associated with persistent bacterial infections includes Clostridium difficile, Enterococcus (e.g., E. faecalis, E. faecium, E. casseliflavus, E. gallinarum, E. raffinosus), Mycobacterium tuberculosis, Mycobacterium avium complex (including Mycobacterium intracellulare and Mycobacterium avium), Mycobacterium smegmatis, Mycoplasms genitalium, Staphylococcus aureus, Streptococcus pyogenes, Streptococcus pneumoniae, and Mycobaterium leprae.

An exemplary list of Gram-negative bacteria that have been shown to possess antibiotic resistance or have been associated with persistent bacterial infections includes Campylobacter, Neisseria gonorrhoeae, Enterobacteriaceae, Klebsiella pneumoniae, Salmonella, Escherichia coli, Acinetobacter, Pseudomonas aeruginosa, Burkholderia pseudomallei. Burkholderia cenocepacia, Helicobacter pylori, and Hemophilus influenza. (See, e.g., Cohen et al. Cell Host & Microbe 13: 632-642, the contents of which are incorporated by reference herein in their entirety.)

Treponema pallidum has also been described as associated with persistent bacterial infections (see Grant and Hung. Virulence 4: 273-283, the contents of which are incorporated by reference herein in their entirety).

The instant disclosure expressly contemplates targeting of any of (or any combination of) the above-listed forms of Gram-positive and/or Gram-negative bacteria, particularly those forms of the above-recited bacteria that possess or are at risk of developing antibiotic tolerance and/or antibiotic resistance.

### Methods of Treatment

The compositions and methods of the present disclosure may be used in the context of a number of therapeutic or prophylactic applications. Compositions of the instant disclosure can be selected and/or administered as a single agent, or to augment the efficacy of another therapy (second therapy), it may be desirable to combine these compositions and methods with one another, or with other agents and methods effective in the treatment, amelioration, or prevention of diseases and/or infections.

In certain embodiments of the instant disclosure, one or more pyrimidine compounds can be administered to a subject, together with administration of an antimicrobial agent for which activity enhancement is desired. It is contemplated that in certain embodiments, one or more pyrimidine compounds of the instant disclosure can be co-administered with an antimicrobial agent for which activity enhancement is desired and/or administration of one or more pyrimidine compounds of the instant disclosure can precede or follow administration of an antimicrobial agent for which activity enhancement is desired. It is also expressly contemplated that the pyrimidine/antimicrobial agent compositions and methods of the instant disclosure can optionally be administered in further combination with other agents, including, e.g., other agents capable of enhancing antimicrobial agent efficacy (such as, e.g., β-lactamase inhibitors, among other antibiotic potentiators/adjuvants that are known in the art).

Administration of a composition of the present disclosure to a subject will follow general protocols for the administration described herein, and the general protocols for the administration of a particular secondary therapy will also be followed, taking into account the toxicity, if any, of the treatment. It is expected that the treatment cycles would be repeated as necessary. It also is contemplated that various standard therapies may be applied in combination with the described therapies.

### Additional Therapeutic Uses

While nucleotide analogues have previously been described for use as anticancer and antiviral chemotherapeutics, their use as potentiators of antiviral agents in non-chemotherapeutic settings is contemplated herein. Exemplary antiviral agents for such uses include, without limitation, Abacavir (use for HIV), Acyclovir (Aciclovir—use for herpes e.g. Chicken pox), Adefovir (use for chronic Hepatitis B), Amantadine (use for influenza), Ampligen, Amprenavir (Agenerase—Use for inhibition of HIV), Arbidol, Atazanavir, Atripla (fixed dose drug), Balavir, Baloxavir marboxil (Xofluza), Biktarvy. Boceprevir (Victrelis), Cidofovir, Cobicistat (Tybost), Combivir (fixed dose drug), Daclatasvir (Daklinza), Darunavir, Delavirdine, Descovy, Didanosine, Docosanol, Dolutegravir, Doravirine (Pifeltro), Ecoliever, Edoxudine, Efavirenz, Elvitegravir, Emtricitabine, Enfuvirtide, Entecavir, Etravirine (Intelence), Famciclovir, Fixed dose combination (antiretroviral), Fomivirsen, Fosamprenavir, Foscanet, Fosfonet, Fusion inhibitor, Ganciclovir (Cytovene), Ibacitabine, Ibalizumab (Trogarzo), Idoxuridine, Imiquimod, Imunovir, Indinavir, Inosine, Integrase inhibitor, Interferon type I, Interferon type II, Interferon type III, Interferon, Lamivudine, Letermovir (Prevymis), Lopinavir, Loviride, Maraviroc, Methisazone, Moroxydine, Nelfinavir, Nevirapine, Nexavir, Nitazoxanide. Norvir, Nucleoside analogues, Oseltamivir (Tamiflu), Peginterferon alfa-2a, Peginterferon alfa-2b, Penciclovir, Peramivir (Rapivab), Pleconaril, Podophyllotoxin, Protease inhibitor (pharmacology), Pyramidine, Raltegravir, Remdesivir, Reverse transcriptase inhibitor. Ribavirin, Rilpivirine (Edurant), Rimantadine, Ritonavir, Saquinavir, Simeprevir (Olysio), Sofosbuvir, Stavudine, Synergistic enhancer (antiretroviral), Telaprevir, Telbivudine (Tyzeka), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine.

Use of the nucleotide analogues of the instant disclosure for treatment of a subject having or at risk of developing a viral infection is also expressly contemplated. Such a subject can have, e.g., an influenza infection, a coronavirus infection (e.g., SARS, MERS, COVID-19), a rhinovirus infection, an HIV infection, etc. In certain embodiments, the subject has or is at risk of developing a lung infection, such as a pneumonia (optionally a viral pneumonia), and the subject is treated with a nucleotide analog (e.g., a pyrimidine) together with an antimicrobial agent and/or an antiviral agent.

### Pharmaceutical Compositions

Agents of the present disclosure can be incorporated into a variety of formulations for therapeutic use (e.g., by administration) or in the manufacture of a medicament (e.g., for treating or preventing a bacterial infection) by combining the agents with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols.

Pharmaceutical compositions can include, depending on the formulation desired, pharmaceutically-acceptable, non-toxic carriers or diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249: 1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977):1-19, incorporated herein by reference. The salts can be prepared in situ during the final isolation and purification of the compounds of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound (e.g., an FDA-approved compound where administered to a human subject) or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moeity advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of certain compounds of the present application which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the application. The term “prodrug” refers to compounds that are rapidly transformed in vivo to yield the parent compound of an agent of the instant disclosure, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, (1987), both of which are incorporated herein by reference.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in a subject and/or tissue of a subject, e.g., to prevent rapid clearance of a formulation by the subject. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent, such as a pyrimidine compound, optionally together with an antimicrobial agent, in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease/infection to be treated and the like. Characteristics of the polymers will include biodegradability at the site of implantation, compatibility with the agent of interest, case of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III., CRC Press, Boca Raton, Fla., 1987, pp 137-149.

### Pharmaceutical Dosages

Pharmaceutical compositions of the present disclosure containing an agent described herein may be used (e.g., administered to an individual, such as a human individual, in need of treatment with a pyrimidine compound, optionally together with an antimicrobial agent) in accord with known methods, such as oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell. W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, or about 2 mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxycthylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such as magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an agent (e.g., a pyrimidine compound, optionally together with an antimicrobial agent) described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Drugs provided herein can be formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for oral delivery or intravenous injection to a subject.

The exact amount of an agent required to achieve an effective amount will vary from subject to subject, depending, for example, on species, age, and general condition of a subject, severity of the side effects or disorder/infection, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent (e.g., a pyrimidine compound, optionally together with an antimicrobial agent) described herein.

As noted elsewhere herein, a drug of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

In some embodiments of the present disclosure, a formulation as herein defined is administered to the subject by bolus administration.

A drug or other therapy of the instant disclosure is administered to the subject in an amount sufficient to achieve a desired effect at a desired site (e.g., reduction of bacterial infection, bacterial abundance, symptoms, etc.) determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Additional exemplary doses for administration of an agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 μg/kg/day.

In certain embodiments, when multiple doses are administered to a subject or applied to a tissue, the frequency of administering the multiple doses to the subject or applying the multiple doses to the tissue is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., a pyrimidine compound, optionally together with an antimicrobial agent) described herein. In certain embodiments, a dose described herein includes independently between 1 mg and 3 mg, inclusive, of an agent (e.g., a pyrimidine compound, optionally together with an antimicrobial agent) described herein. In certain embodiments, a dose described herein includes independently between 3 mg and 10 mg, inclusive, of an agent (e.g., a pyrimidine compound, optionally together with an antimicrobial agent) described herein. In certain embodiments, a dose described herein includes independently between 10 mg and 30 mg, inclusive, of an agent (e.g., a pyrimidine compound, optionally together with an antimicrobial agent) described herein. In certain embodiments, a dose described herein includes independently between 30 mg and 100 mg, inclusive, of an agent (e.g., a pyrimidine compound, optionally together with an antimicrobial agent) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent (e.g., a pyrimidine compound, optionally together with an antimicrobial agent) or composition, as described herein, can be administered in combination with one or more additional pharmaceutical agents (e.g., therapeutically and/or prophylactically active agents), which are different from the agent or composition and may be useful as, e.g., combination therapies.

The agents or compositions can be administered in combination with additional pharmaceutical agents that improve their activity (e.g., activity (e.g., potency and/or efficacy) in treating a disease or infection (e.g., a bacteremia and/or an antibiotic-tolerant or antibiotic-resistant bacterial infection) in a subject in need thereof, in preventing a disease or infection in a subject in need thereof, in reducing the risk of developing a disease or infection in a subject in need thereof, etc. in a subject or tissue. In certain embodiments, a pharmaceutical composition described herein including an agent (e.g., a pyrimidine compound, optionally together with an antimicrobial agent) described herein and an additional pharmaceutical agent shows a synergistic effect that is absent in a pharmaceutical composition including one of the agent and the additional pharmaceutical agent, but not both.

In some embodiments of the disclosure, a therapeutic agent distinct from a first therapeutic agent of the disclosure is administered prior to, in combination with, at the same time, or after administration of the agent of the disclosure. In some embodiments, the second therapeutic agent is selected from the group consisting of a chemotherapeutic, an immunotherapy, an antioxidant, an anti-inflammatory agent, an antimicrobial, a steroid, etc.

The agent or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease or infection described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, additional antibiotics, antimicrobials, anti-proliferative agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents.

Dosages for a particular agent of the instant disclosure may be determined empirically in individuals who have been given one or more administrations of the agent.

Administration of an agent of the present disclosure can be continuous or intermittent, depending, for example, on the recipient's physiological condition, whether the purpose of the administration is therapeutic or prophylactic, and other factors known to skilled practitioners. The administration of an agent may be essentially continuous over a preselected period of time or may be in a series of spaced doses.

Guidance regarding particular dosages and methods of delivery is provided in the literature, see, for example, U.S. Pat. Nos. 4,657,760; 5,206,344; or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Kits

The instant disclosure also provides kits containing agents of this disclosure for use in the methods of the present disclosure. Kits of the instant disclosure may include one or more containers comprising an agent (e.g., a pyrimidine compound, optionally together with an antimicrobial agent and/or an antiviral agent) and/or composition (e.g., a pyrimidine compound, optionally together with an antimicrobial agent and/or an antiviral agent) of this disclosure. In some embodiments, the kits further include instructions for use in accordance with the methods of this disclosure. In some embodiments, these instructions include a description of administration of the agent to treat or prevent, e.g., an infection and/or disease. In some embodiments, the instructions include a description of how to administer a pyrimidine and/or an antimicrobial agent to a bacterial population and/or to a subject, in certain embodiments to a subject infected or suspected to be infected or at risk of infection with a bacteria and/or a virus. In some embodiments, the kit further includes an agent (or instructions) for measuring prpp accumulation and/or an agent (or instructions) for measuring the lethality of the antimicrobial agent and/or the antiviral agent against a target microbe and/or a target virus.

The instructions generally include information as to dosage, dosing schedule, and route of administration for the intended use/treatment. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., scaled Mylar or plastic bags), and the like. The container may further include a pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor. N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press. London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Bacterial Strains, Media, Growth Conditions, and Reagents

Escherichia coli strain K-12 MG1655 (ATCC 700926) was used for all experiments of the instant Examples. For metabolite supplementation experiments, cells were cultured in MOPS minimal medium with 0.2% glucose (Teknova; Hollister, Calif.). For experiments involving gene deletions, cells were cultured in MOPS EZ Rich defined medium (Teknova). For all experiments, cells were grown at 37° C. either on a rotating shaker at 300 rpm in baffled flasks or 14 mL test tubes or on a rotating shaker at 900 rpm in Biolog 96-well phenotype microarrays (Bochner, 2009) (Biolog; Hayward, Calif.). All experiments were performed with n≥3 biological replicates from independent overnight cultures. Uniformly labeled ¹³C glucose was purchased from Cambridge Isotope Laboratories, Inc. (Tewksbury, Mass.). LC-MS reagents were purchased from Honeywell Burdick & Jackson® (Muskegon, Mich.) and Sigma-Aldrich (St. Louis, Mo.).

### Metabolite Screen and IC₅₀ Determination

An overnight culture of E. coli cells in MOPS minimal medium was diluted 1:500 and grown to mid-exponential phase at 37° C. with 300 rpm shaking in 2 L baffled flasks. 13 mL cultures were then back-diluted to OD₆₀₀=0.1 and dispensed into 14 mL test tubes containing 100× concentrated AMP, CIP or GENT over the following concentration gradients: for AMP, 10 mg/mL, 1 mg/mL and 1.5-fold dilutions from 20-0.35 μg/mL; for CIP, 10 μg/mL, 1 μg/mL and 1.5-fold dilutions from 100-0.4 ng/mL; for GENT, 10 μg/mL, 1 μg/mL and 1.5-fold dilutions from 200-2.6 ng/mL. 100 μL from each antibiotic-treated subculture was dispensed into each well of a Biolog PM 1-4 compound plate. Plates were sealed with breathable membranes and incubated in a 37° C. shaking incubator with 900 rpm shaking. After 4 h incubation, OD₆₀₀ was measured on a SpectraMax® M5 Microplate Reader (Molecular Devices®; San Jose, Calif.). IC₅₀s were estimated from each set of n≥3 independent biological replicates by fitting logistic functions to each set of OD₆₀₀ measurements for each well in MATLAB® (Mathworks®; Natick, Mass.). In the case of CIP, some metabolite conditions exhibited a biphasic dose-response. For those conditions, a logistic function was fit to only the phase at the lower concentration.

### Gene Knockout Strain Construction

E. coli ΔglyA, ΔpurD, ΔpurE, ΔpurK, ΔpurM, ΔpurC and ΔpyrE gene deletion mutants were constructed by P1 phage transduction using the Keio collection (Baba et al., 2006), as previously described (Gutierrez et al., 2017). Briefly, P1 phage lysates corresponding to each gene deletion were produced by incubating overnight cultures of Keio donor strains with P1 phage. For each gene deletion, an overnight culture of E. coli MG1655 was pelleted and resuspended in a 10 mM MgCl₂ and 5 mM CaCl₂ salt solution in a 15 mL test tube, and then incubated with the corresponding P1 phage at 37° C. for 30 min. Media containing 1 M sodium citrate was added to each tube and incubated at 37° C. for an additional 60 min in a 300 rpm shaking incubator. Cells were pelleted, resuspended on fresh media, and then plated on kanamycin-selective agar plates containing 5 mM sodium citrate and incubated overnight at 37° C. Colonies were selected from each plate and their kanamycin-resistance cassettes cured by transducing pCP20 plasmid with electroporation, inducing recombination by overnight growth at 43° C., and then screening resulting colonies for genomic recombination and plasmid loss on kanamycin- and ampicillin-selective agar plates. Overnight cultures of each knockout strain were checked for accuracy by PCR amplification and gel electrophoresis with custom oligonucleotides (Table 9).

### Time-Kill Experiments

Time-kill experiments were performed as previously described (Dwyer et al., 2014). An overnight culture of E. coli cells in MOPS minimal medium was diluted 1:500 and grown to mid-exponential phase at 37° C. with 300 rpm shaking in 125 mL baffled flasks. 1 mL cultures were then back-diluted to OD₆₀₀=0.1, dispensed into 14 mL test tubes and treated with AMP, CIP or GENT, with biochemical supplementation where indicated. For all metabolite supplementation experiments in minimal media, time-kill experiments were performed using 4 μg/mL AMP, 16 ng/mL CIP or 48 ng/mL GENT. For all gene knockout experiments in rich media, time-kill experiments were performed using 4 μg/mL AMP, 16 ng/mL CIP or 96 ng/mL GENT. Hourly samples were collected and serially diluted in PBS for colony enumeration 24 h later.

### Intracellular Metabolite Quantification

Intracellular metabolites were quantified on an AB SCIEX Qtrap® 5500 mass spectrometer (AB SCIEX; Framingham, Mass.), as previously described (McCloskey et al., 2018), and processed using in house scripts. An overnight culture of E. coli cells in MOPS minimal medium was diluted 1:500 and grown to mid-exponential phase at 37° C. with 300 rpm shaking in 1 L baffled flasks. 25 mL cultures were then back-diluted to OD₆₀₀=0.1, dispensed into 250 mL baffled flasks and treated with either 1 mM adenine, 1 mM uracil or a non-supplemented control. Samples were collected 1 hr after supplementation, and aliquots with biomass equivalents to 10 mL of cell culture at OD₆₀₀=0.1 were subjected to metabolite extraction using a 40:40:20 mixture of acetonitrile, methanol and LC-MS grade water. Uniformly labeled ¹³C-standards were generated by growing E. coli in uniformly labeled Glucose M9 minimal media in aerated shake flasks, as previously described (McCloskey et al., 2014). Calibration mixes of standards were split across several mixes, aliquoted, and lyophilized to dryness. All samples and calibrators were equally spiked with the same internal standards. Samples were quantified using isotope-dependent mass spectrometry. Calibration curves were run before and after all biological and analytical replicates. Consistency of quantification between calibration curves was checked by running a Quality Control sample composed of all biological replicates. Values reported are derived from the average of the biological triplicates, analyzed in duplicate (n=6).

### Oxygen Consumption Rate Quantification

Bacterial respiratory activity was quantified using the Seahorse XF^{e}96 Extracellular Flux Analyzer (Seahorse Bioscience; North Billerica, Mass.), as previously described (Dwyer et al., 2014; Lobritz et al., 2015). XF Cell Culture Microplates were pre-coated with 100 ng/mL poly-D-lysine. An overnight culture of E. coli cells in MOPS minimal medium was diluted 1:500 and grown to mid-exponential phase at 37° C. with 300 rpm shaking in 125 mL baffled flasks. Cells were back-diluted to OD₆₀₀=0.01 and 90 μL diluted cells were dispensed to each well of the coated XF Microplates. Microplates were centrifuged for 10 min at 4,000 rpm and an additional 90 μL fresh media with or without 1 mM adenine or uracil was added to each well. Antibiotics were added to injection ports and measurements taken at 5 min intervals with 2.5 min measurements cycles and 2.5 min mixing.

### Hierarchical Clustering

Hierarchical clustering for the measured antibiotic IC₅₀s and identified pathways was performed in MATLAB® using the standardized Euclidean distance metric.

### Metabolite Set Enrichment Analysis

Metabolite Set Enrichment Analysis was performed in Ecocyc (v. 22.0) (Keseler et al., 2017). A SmartTables was created comprised of metabolites eliciting a ≥2-fold change in IC₅₀ for at least one antibiotic (Table 4). Pathways were identified using the “Enrichment” analysis type. The Fisher Exact test was performed for each enrichment analysis with false discovery rate (FDR) correction by the Benjamini-Hochberg method.

### Genome-Scale Metabolic Modeling

Metabolic simulations were performed using the COBRA Toolbox v. 2.0 (Schellenberger et al., 2011) in MATLAB and Gurobi Optimizer v. 6.0.4 (Gurobi Optimization; Beaverton, Oreg.). Reversible reactions in the iJO1366 E. coli model (Orth et al., 2011) were replaced with pairs of forward and backward reactions. In order to simulate growth in MOPS minimal medium, reaction bounds from the exchange reactions corresponding to each metabolite present in MOPS minimal medium were set to a value of ‘1,000’, to permit uptake. Reaction bounds for oxygen exchange, glucose exchange and cobalamin exchange were as set to values of ‘18.5’, ‘10’ and ‘0.1’, respectively, as previously described (Orth et al., 2011). For each metabolite screening condition, additional exchange reactions were added to represent supplementation with each metabolite on the Biolog phenotype microarray plates (Table 1), with reaction bounds set to ‘1,000’ to permit uptake. Parsimonious flux balance analysis (Lewis et al., 2010) was performed on each metabolite condition-specific model 10,000 times with sampling by optGpSampler (Megchelenbrink et al., 2014). For each reaction in the condition-specific models, the mean flux across all 10,000 samples was computed and used to represent flux in each condition.

### Multitask Elastic Net Regularization

Metabolic reactions for each antibiotic were selected using a two-stage multitask elastic net regularization (Yuan et al., 2016; Zou and Hastie, 2005) in the open-source Spyder IDE v. 3.3.0 (Spyder Project Contributors) Python environment. First, IC₅₀s from each screening condition were normalized by their on-plate controls and log 2-transformed. Multitask elastic net was jointly performed on the transformed antibiotic IC₅₀s and the simulated metabolic states using the MultitaskElasticNetCV function in the scikit-learn toolbox v. 0.17.0 (Pedregosa et al., 2011) with 50-fold cross-validation, 1e4 max iterations and tolerance of 1e-6. Second, for each antibiotic, the standard deviation of elastic net coefficients was computed. Reactions whose coefficients possessed magnitude less than half the standard deviation were filtered and removed. Exchange and transport reactions were excluded from this analysis.

### Hypergeometric Pathway Identification

Pathways mechanisms were identified by performing hypergeometric statistical testing on metabolic pathways curated in Ecocyc (v. 22.0) (Keseler et al., 2017). For each antibiotic, reactions selected by multitask elastic net regularization were converted to their Ecocyc counterparts and hypergeometric p-values were computed for each pathway-reaction set in Spyder. For each antibiotic-pathway combination. FDR statistics were estimated using the Benjamini-Hochberg method. Pathways that exhibited p<0.05 and q<0.0.05 for at least one antibiotic were selected.

### Pathway and Reaction Score Computation

For each antibiotic, log₂-transformed IC₅₀s were regressed on the reactions selected by multitask elastic net by linear squares using scikit-learn in Spyder. For each pathway, pathway scores were computed by first computing the average of the non-zero regression coefficients for all reactions in each pathway. The magnitudes for these pathway scores were then log₁₀-transformed and normalized by the largest magnitude of all pathway scores. Reaction scores were computed by taking the log₁₀-transformation of each regression coefficient for each antibiotic. The magnitudes of these reaction scores were then normalized by the largest magnitude of all reaction scores.

### Metabolite Quantification

Metabolite concentrations were estimated from LC-MS/MS peak heights using previously generated calibration curves. Metabolites found to have a quantifiable variability (RSD≥50%) in the Quality Control samples or possessing individual components with a RSD≥80% were excluded from analysis. Metabolites in blanks with a concentration greater than 80% of that found in the biological samples were similarly excluded. Missing values were imputed by bootstrapping using the R package Amelia II (v. 1.7.4, 1,000 imputations) (Honaker et al., 2011). Remaining missing values were approximated as ½ the lower limit of quantification for the metabolite normalized to the biomass of the sample. Intracellular metabolite concentrations were calculated based on an estimated cell density of 7·10⁷ CFU/mL at OD₆₀₀=0.1 (FIGS. 5A-5C) and an estimated cell volume of 1.3 fL for non-stressed exponential phase E. coli cells (Milo and Phillips, 2016).

### Statistical Analysis

Statistical significance testing was performed in Prism v8.0.2 (GraphPad; San Diego, Calif.). One-way ANOVA was performed on intracellular ATP measurements. Reported p-values reflect false-discovery correction by the Holm-Šidák multiple comparisons test, with comparisons only between adenine or uracil supplementation with control. Although ANOVA is generally robust against lack of normality in the data, statistical tests were not specifically performed to determine if all of the assumptions of ANOVA had been met.

### Example 2: A White-Box Learning Approach for Revealing Metabolic Mechanisms of Antibiotics Lethality

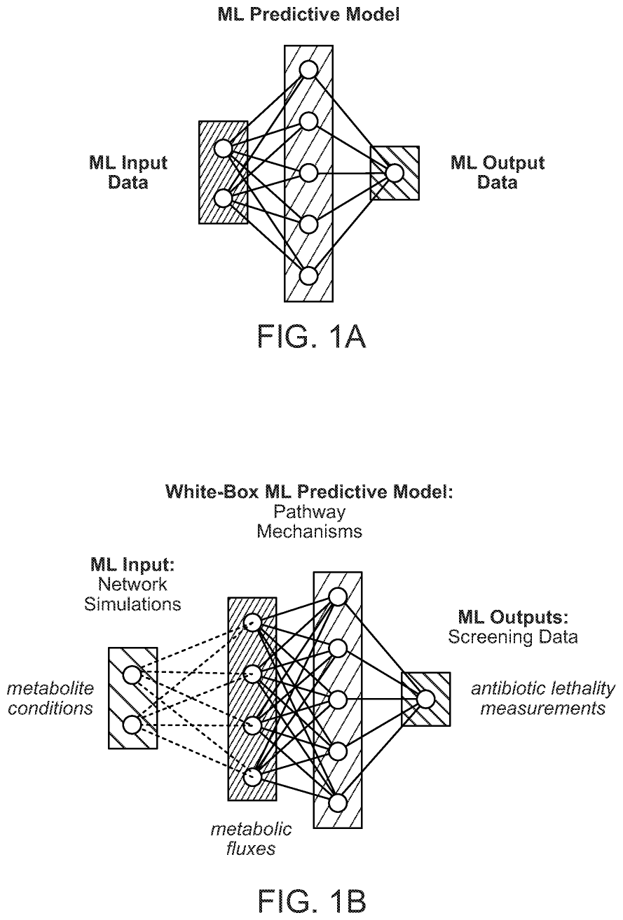

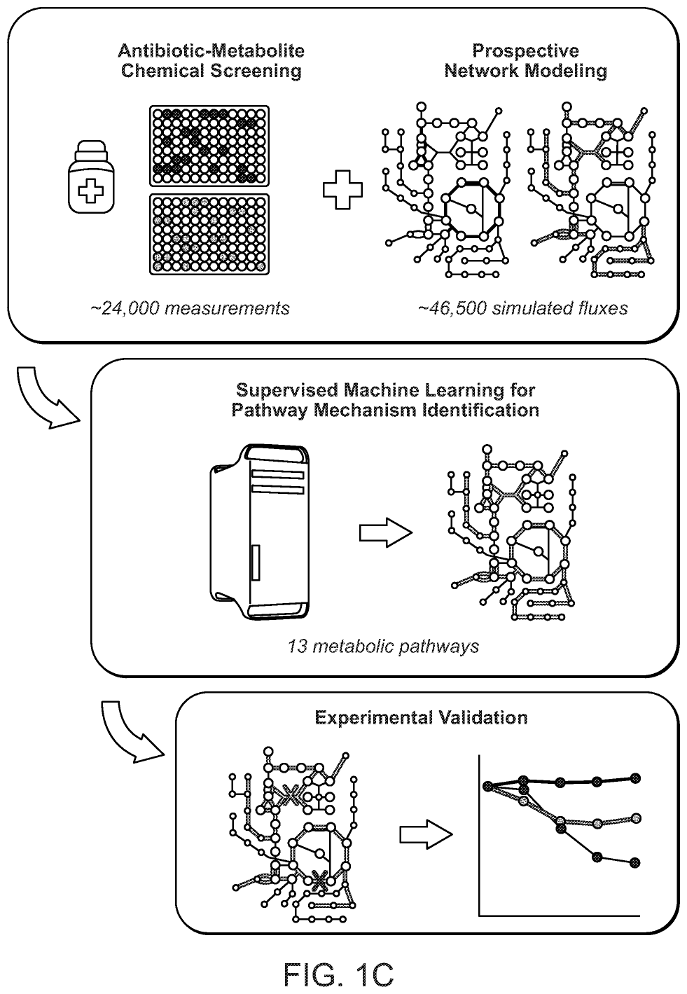

Machine learning aims to generate predictive models from sets of training data; such activities are typically comprised of three parts: input data, output data, and the predictive model trained to compute output data from input data (FIG. 1A) (Camacho et al., 2018). While modern machine learning methods can assemble high-fidelity input-output associations from training data, the functions comprising the resulting trained models often do not possess tangible biochemical analogs, rendering them mechanistically uninterpretable. Consequently, predictive models generated by such (black-box) machine learning activities are unable to provide direct mechanistic insights into how biological molecules are interacting to give rise to observed phenomena. In order to address this limitation, a “white-box” machine learning approach, leveraging carefully curated biological network models to mechanistically link input and output data, was developed (Yu et al., 2018b) and has been implemented herein.

The approach detailed herein has integrated biochemical screening with prospective network modeling to provide mechanistically linked training data for machine learning (FIG. 1B). In contrast to existing approaches, which generate predictive models from only the variables/perturbations available in a screen, prospective network modeling was employed herein to quantitatively transform screening perturbations into biologically enriched network states. Biological information from experimental screens were applied as boundary conditions for the network simulations, computing a network representation of each perturbation in the screen (e.g., metabolic fluxes following metabolite perturbations). These network representations were then used as input data to train predictive models with the empirical screening measurements (e.g., quantified cellular phenotypes in response to screening perturbations) as output data. Because biological networks are mechanistically constructed, the features comprising the predictive models trained by machine learning are, by definition, mechanistically causal and represent tangible biochemical species that can be directly tested experimentally.

This integrated screening-modeling-learning approach was applied towards investigating metabolic mechanisms of antibiotic lethality, demonstrating the ability of this workflow to reveal new mechanistic insights (FIG. 1C). Specifically, biochemical screens were designed to measure the effects of diverse metabolite supplementations on the lethality of three bactericidal antibiotics: ampicillin (AMP, a β-lactam), ciprofloxacin (CIP, a fluoroquinolone) and gentamicin (GENT, an aminoglycoside). Combinations of these antibiotics and metabolites were screened in Escherichia coli, measuring their antibiotic half-maximal inhibitory concentrations (IC₅₀s) after four hours of treatment. Next, metabolic network states were prospectively simulated corresponding to each metabolite perturbation using the iJO1366 genome-scale model of E. coli metabolism (Orth et al., 2011) with quantitative information from the biochemical screens as modeling constraints. These simulations comprehensively yield flux estimates for each metabolic reaction in E. coli, under each screening condition. For each antibiotic, machine learning regression analyses were applied to train a predictive model that could reveal pathway mechanisms underlying differences in antibiotic lethality measured in the screen discussed in this application. These pathways were identified by regularizing the simulated metabolic network states, regressing the measured IC₅₀s and performing enrichment analyses from metabolic pathway annotations curated in Ecocyc v. 22.0 (Keseler et al., 2017).

### Example 3: Exogenous Metabolites Exerted Pathway-Specific Effects on Antibiotic Lethality

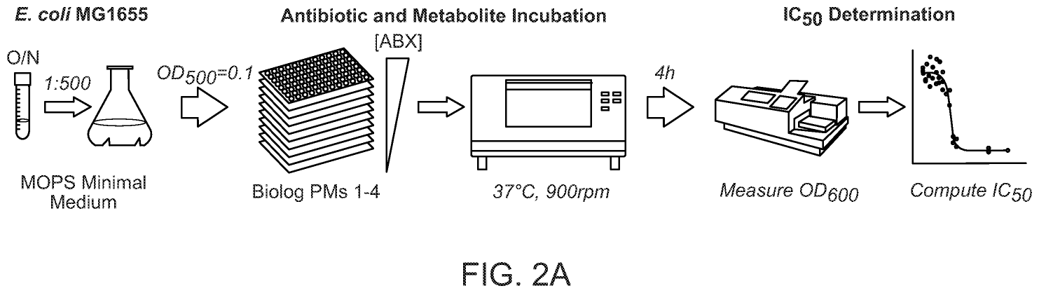

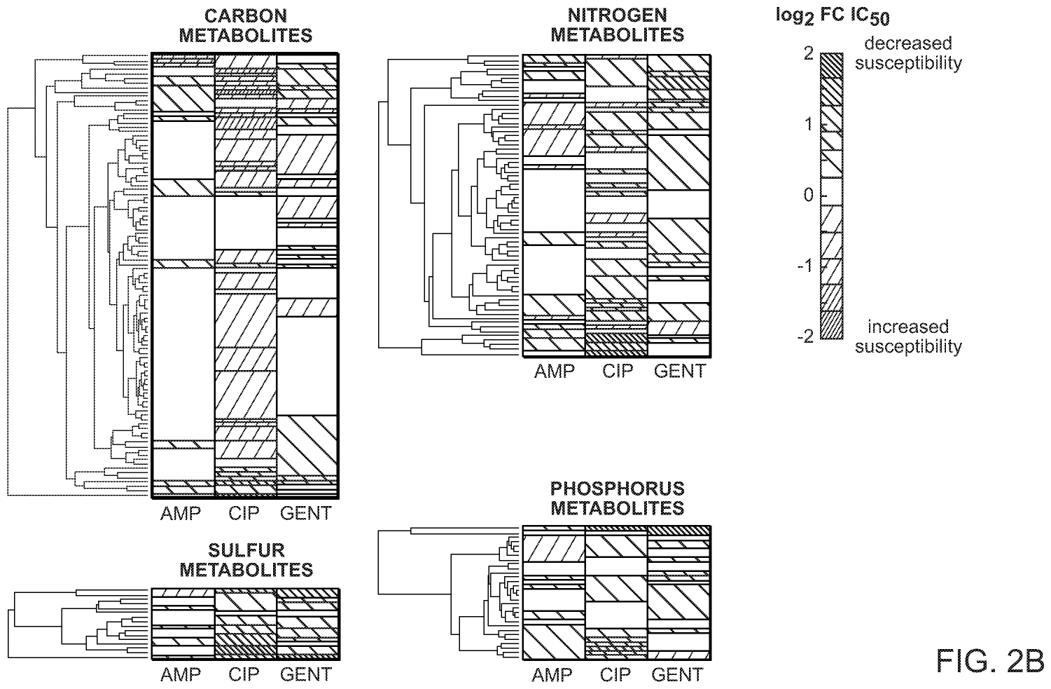

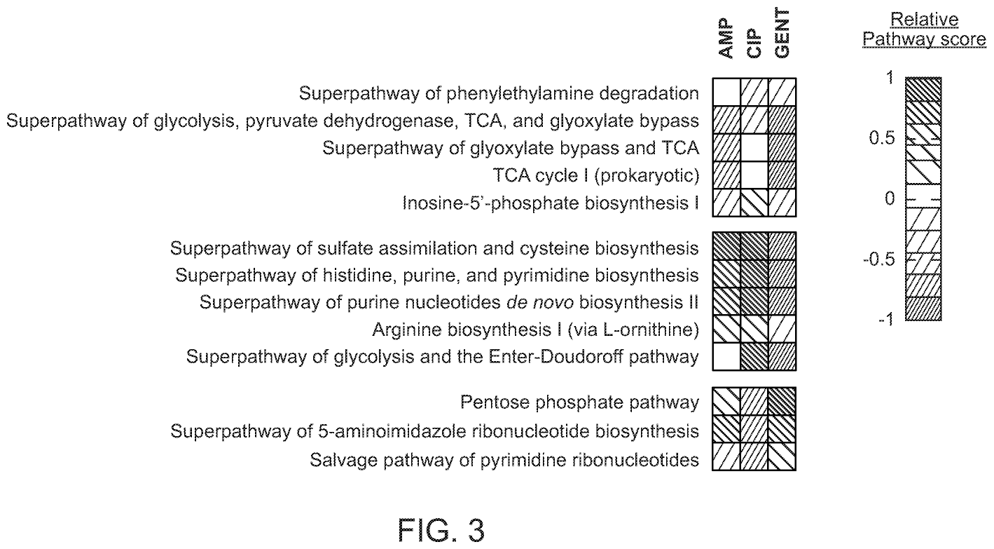

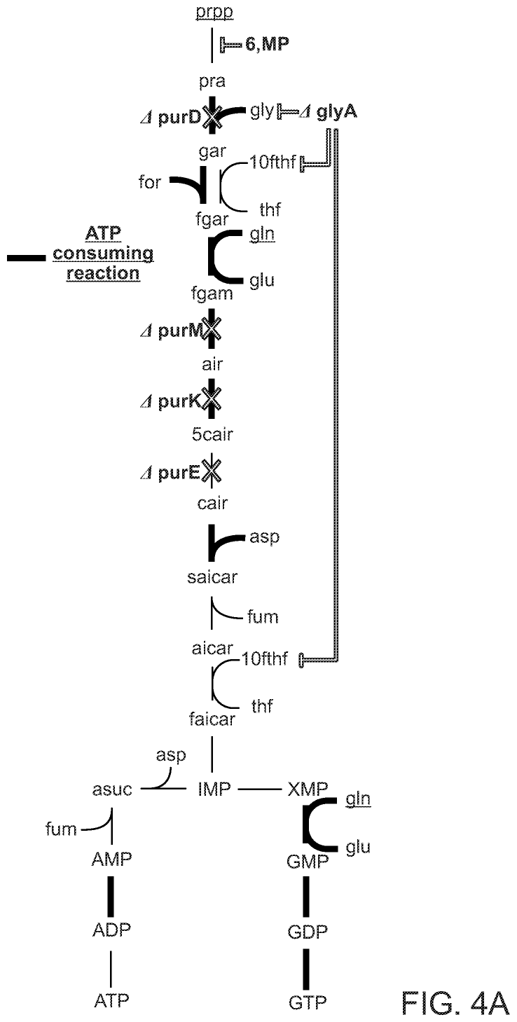

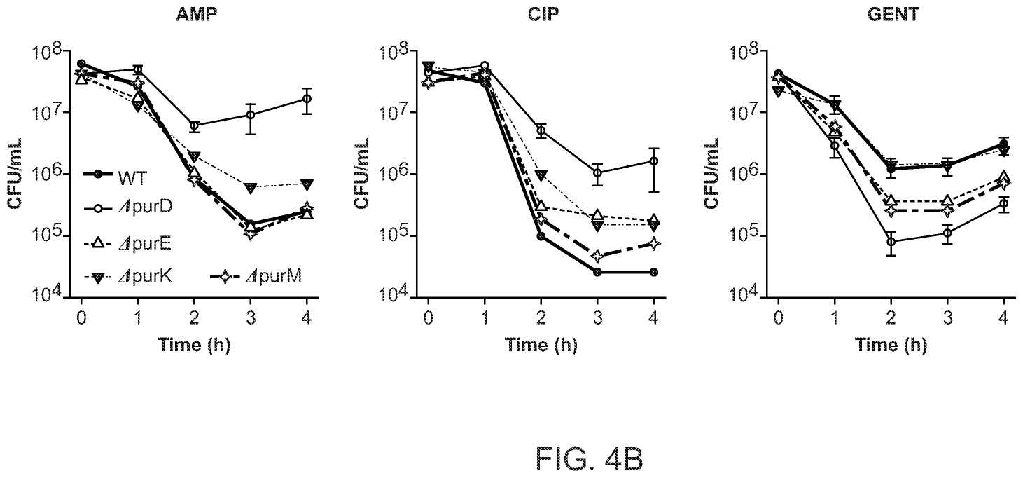

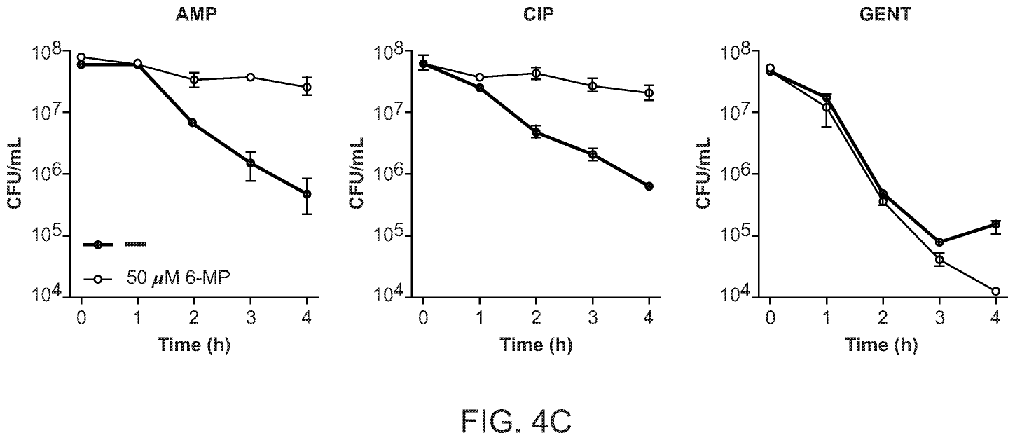

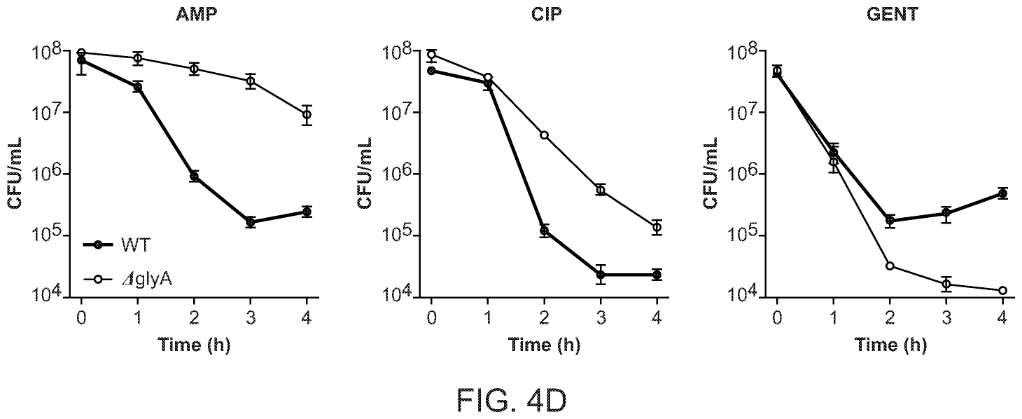

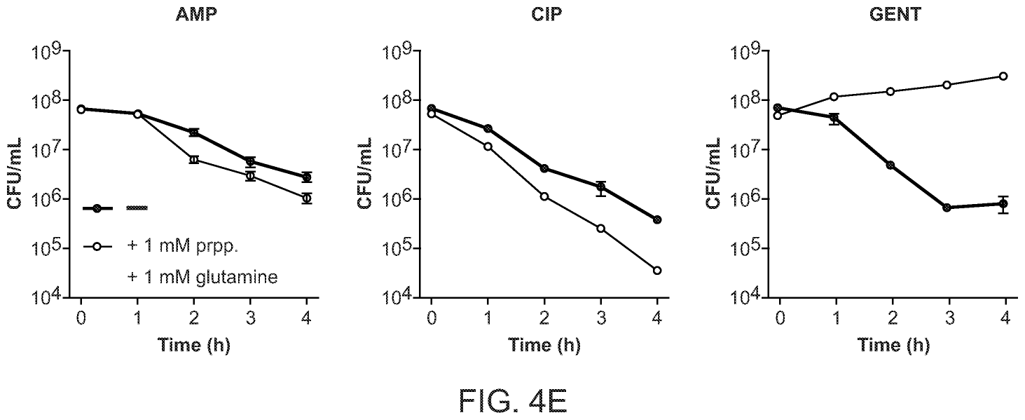

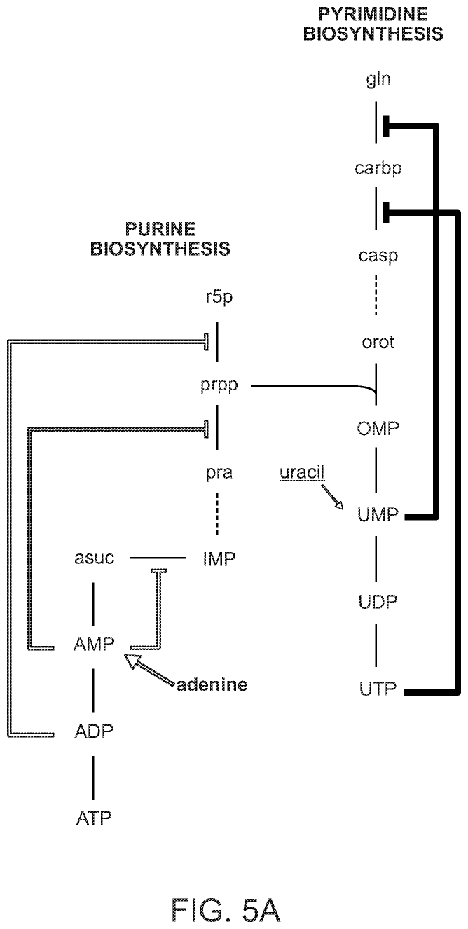

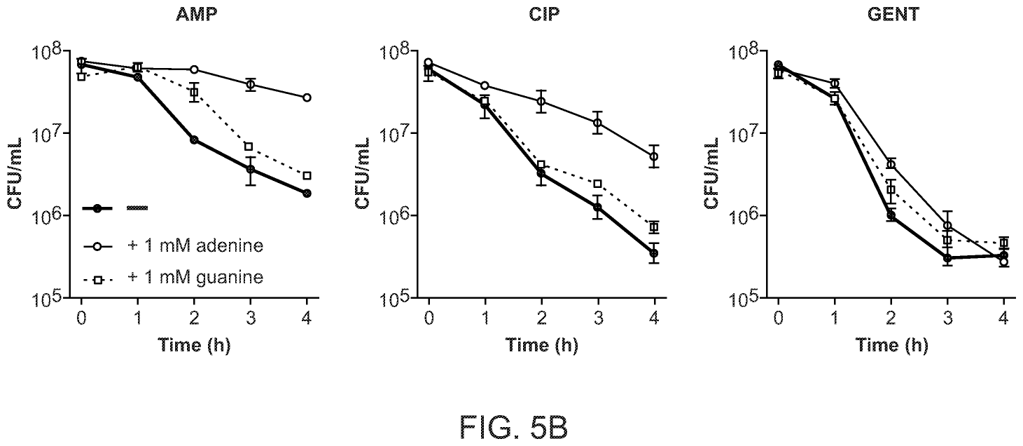

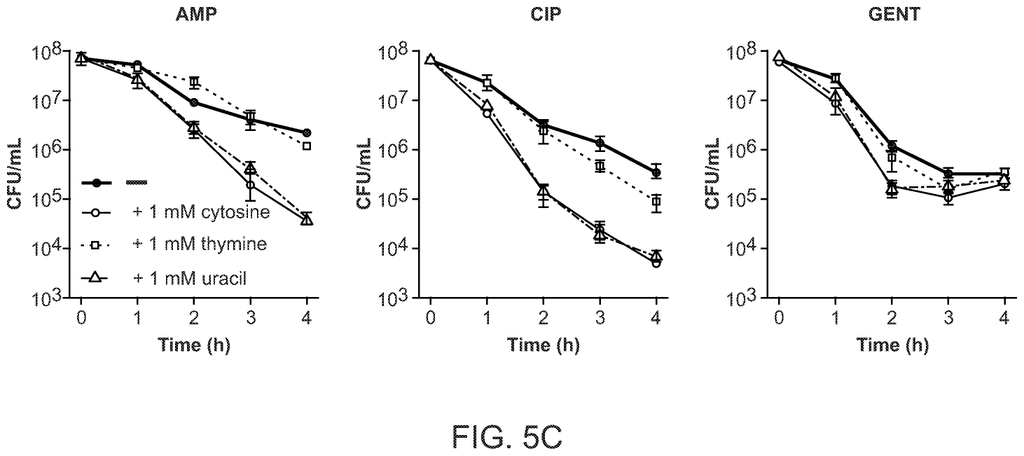

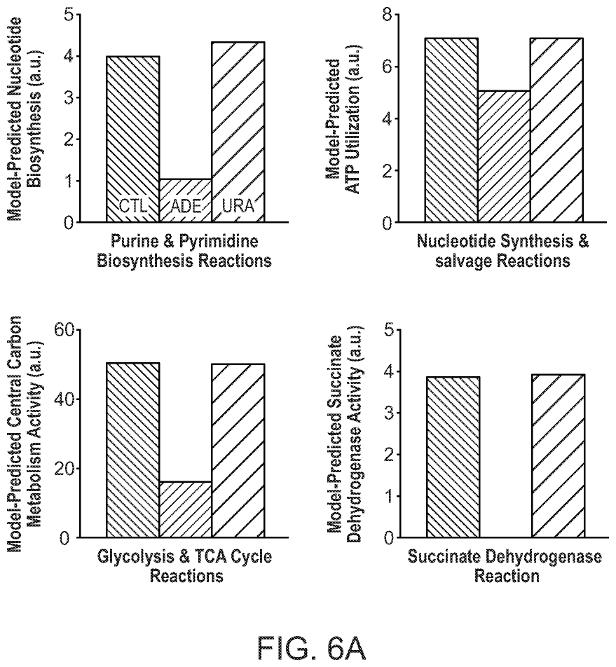

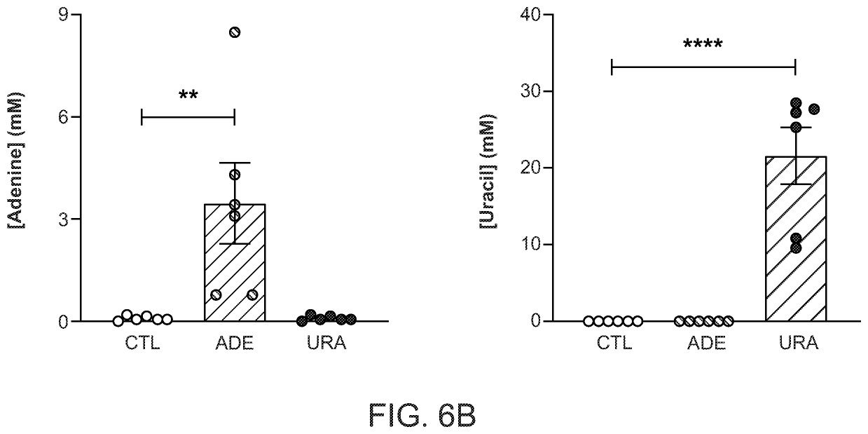

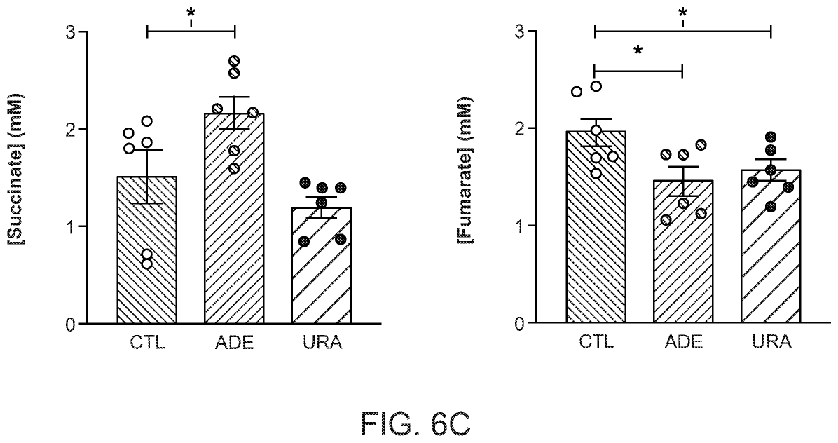

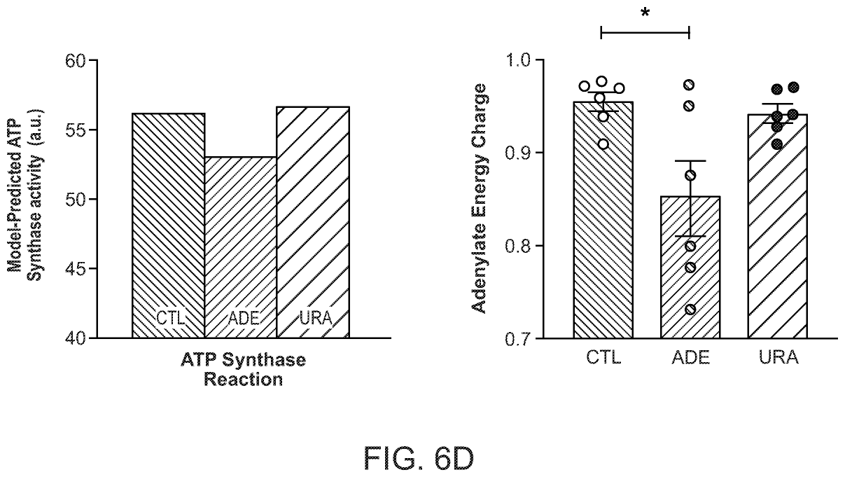

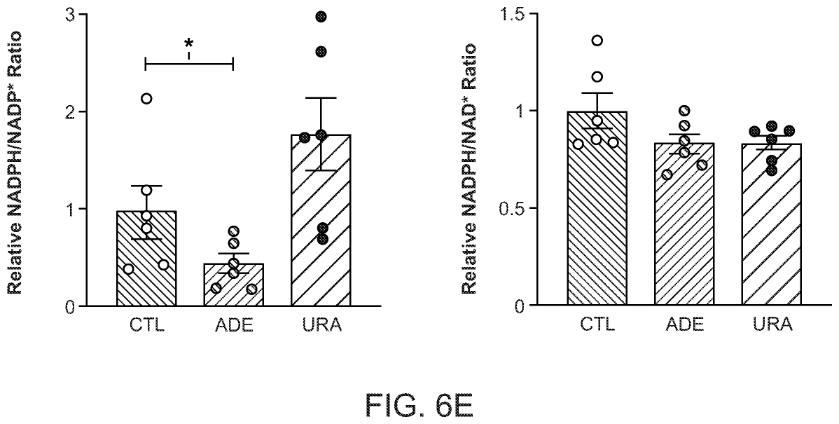

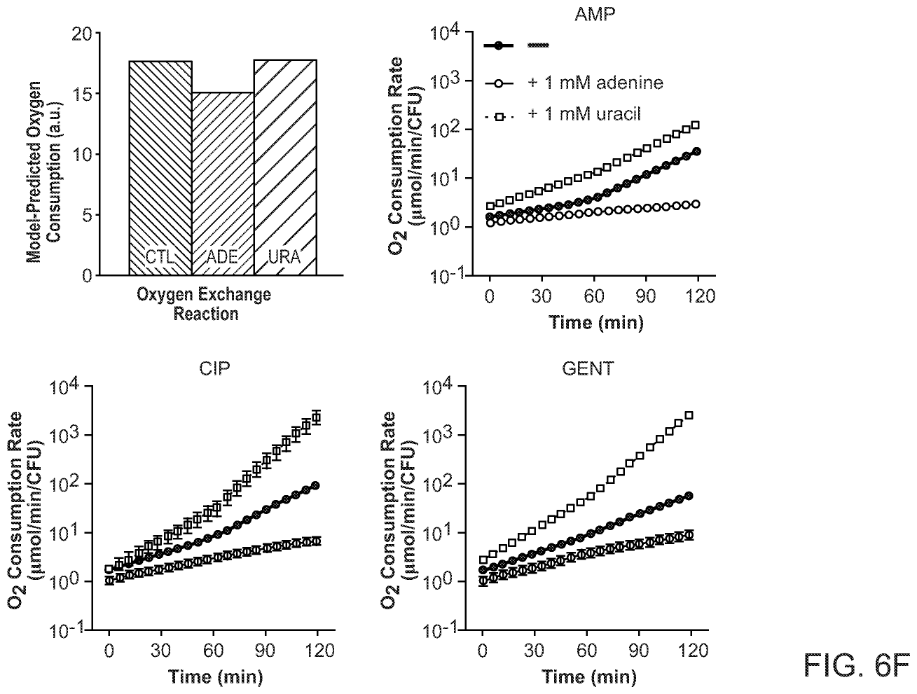

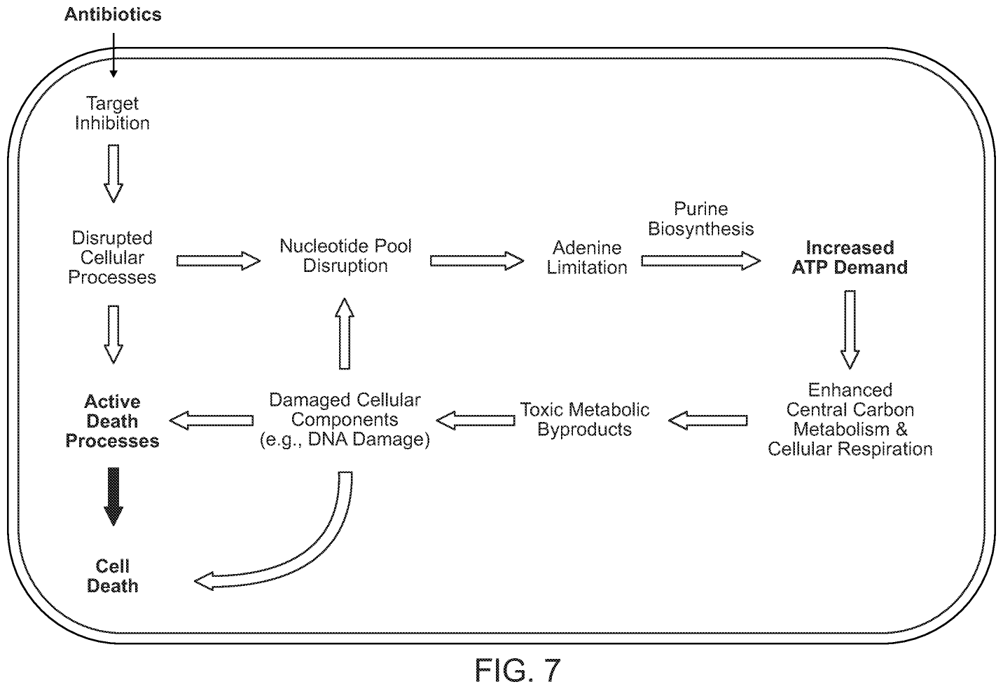

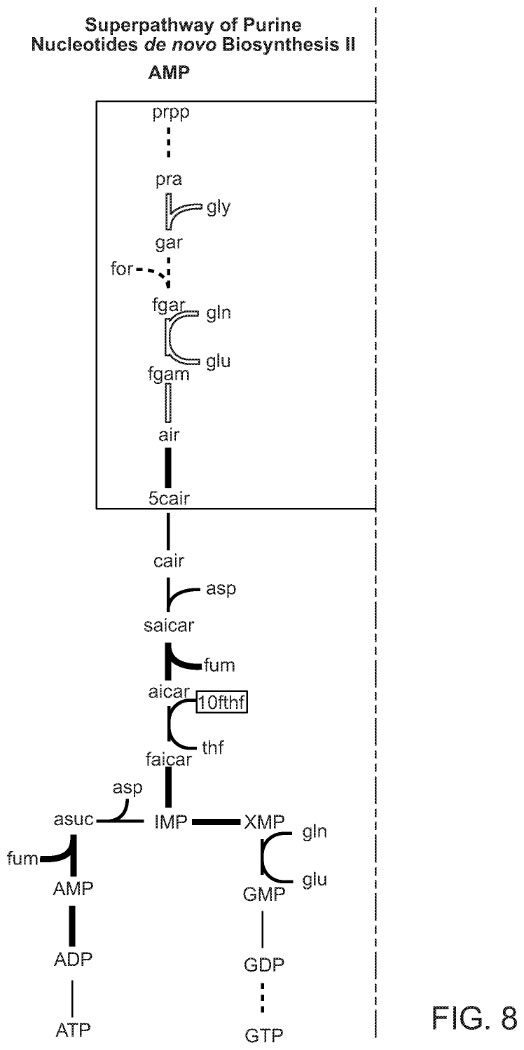

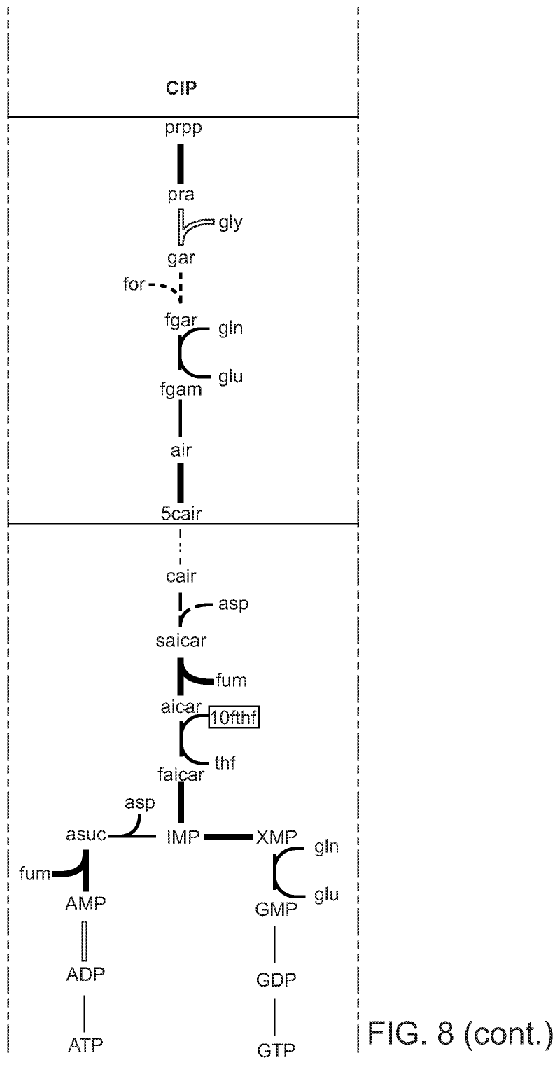

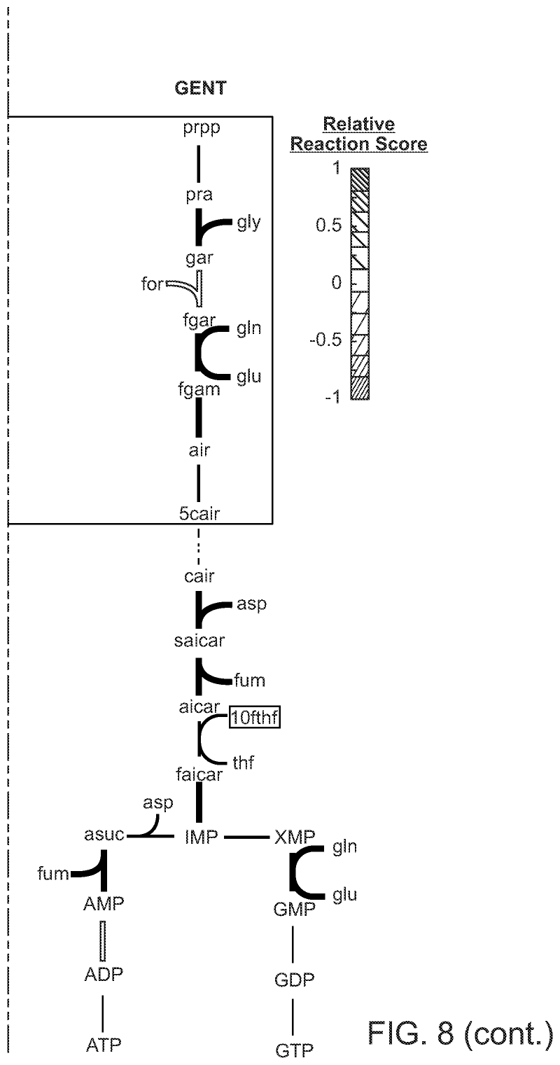

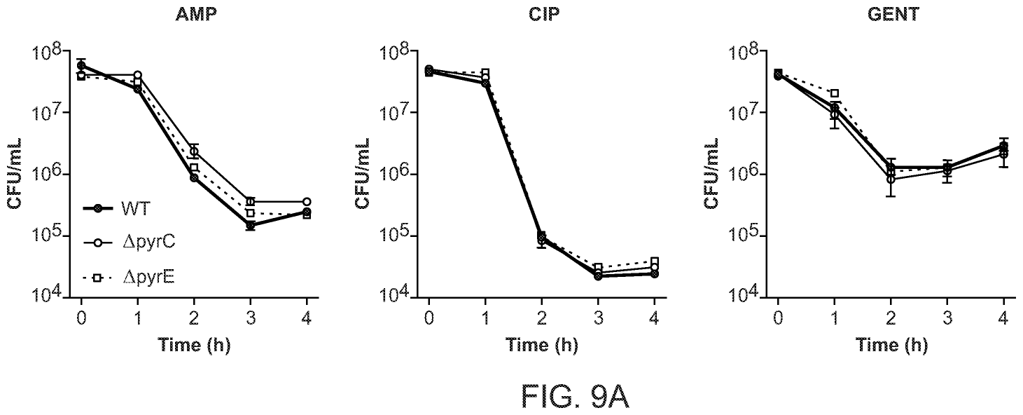

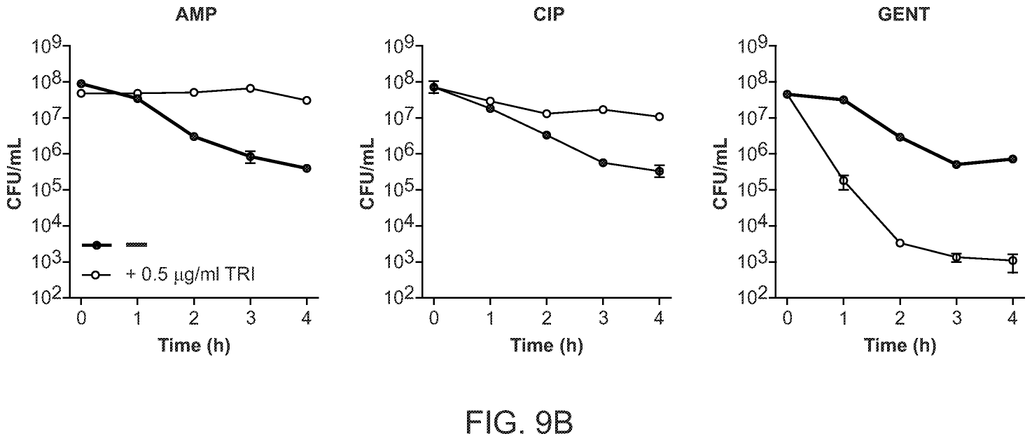

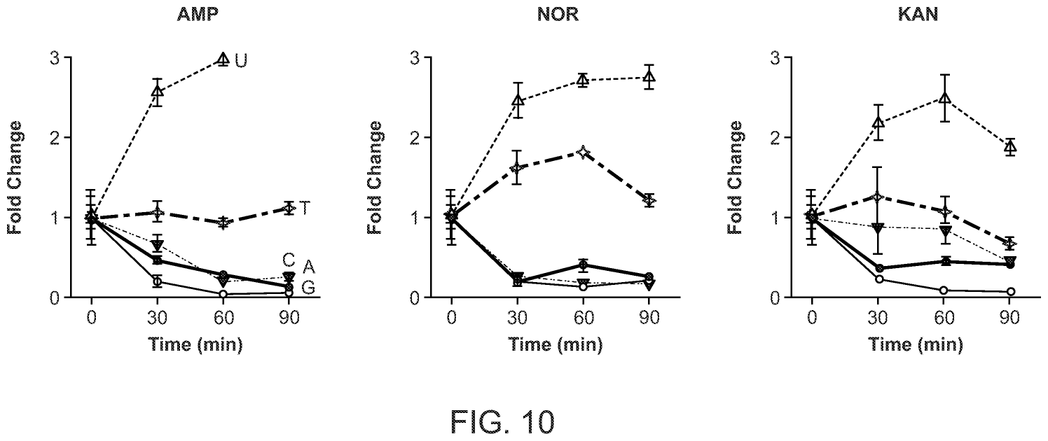

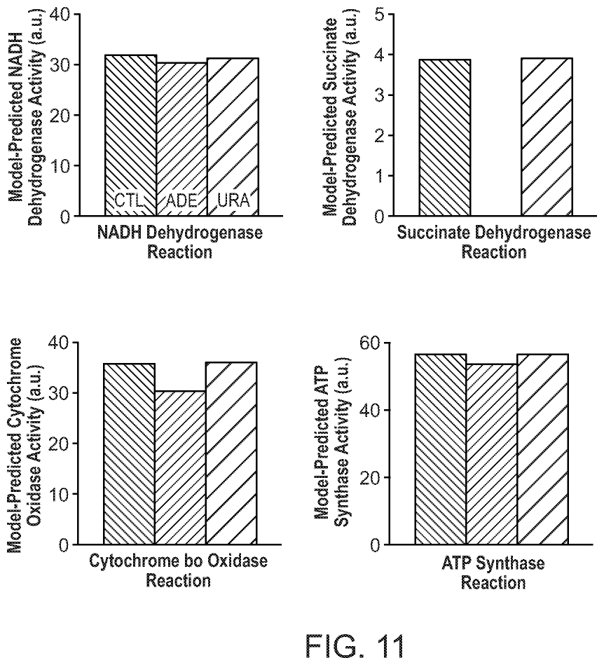

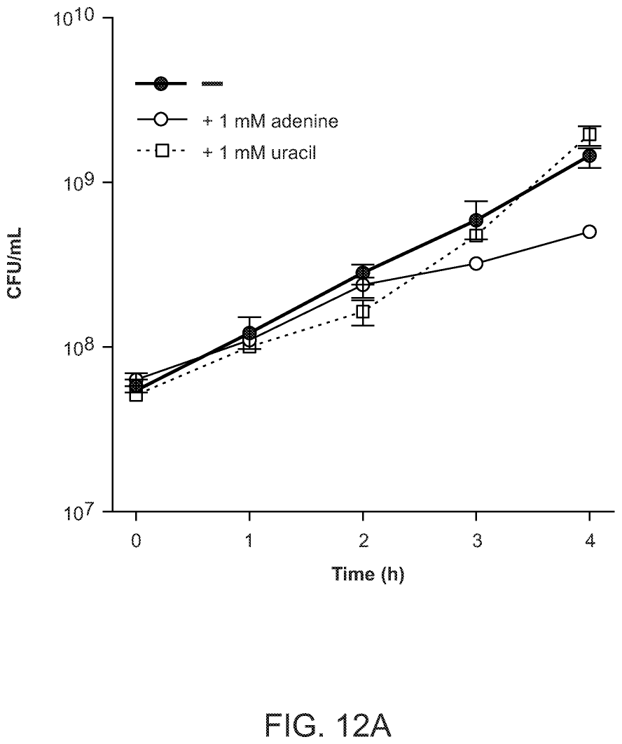

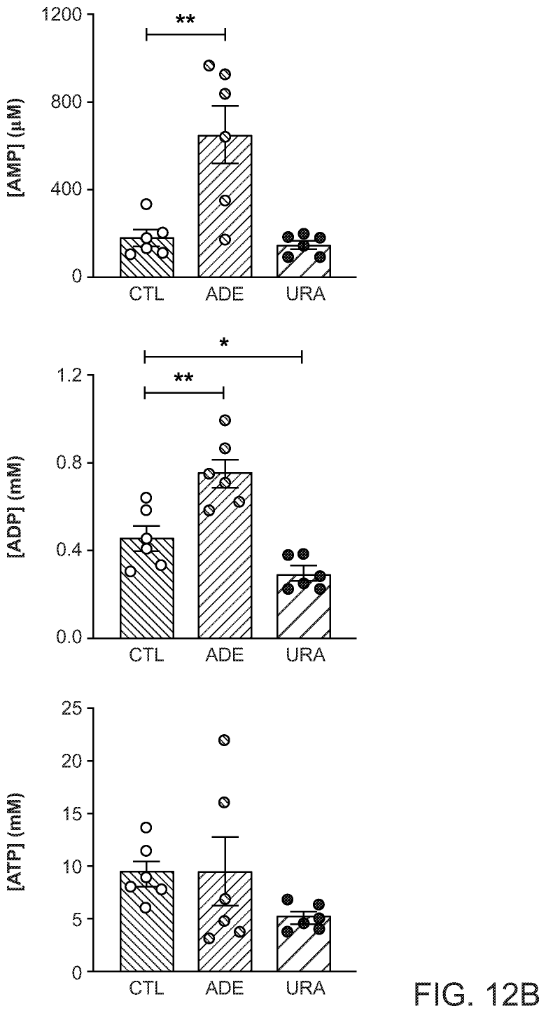

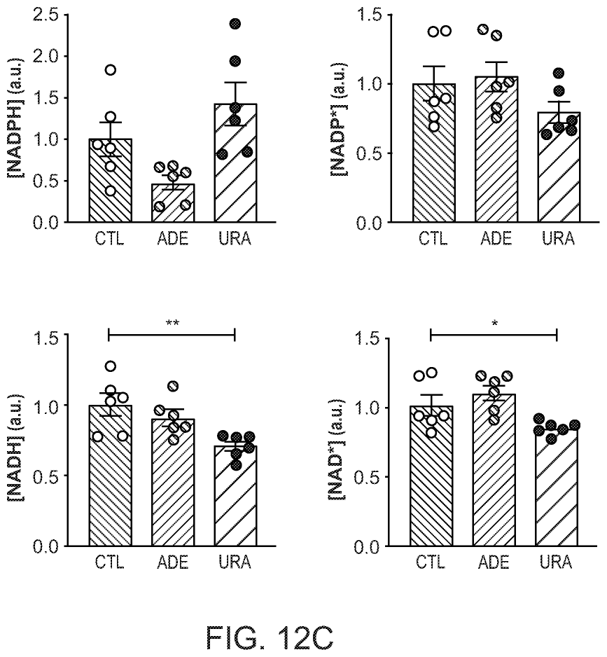

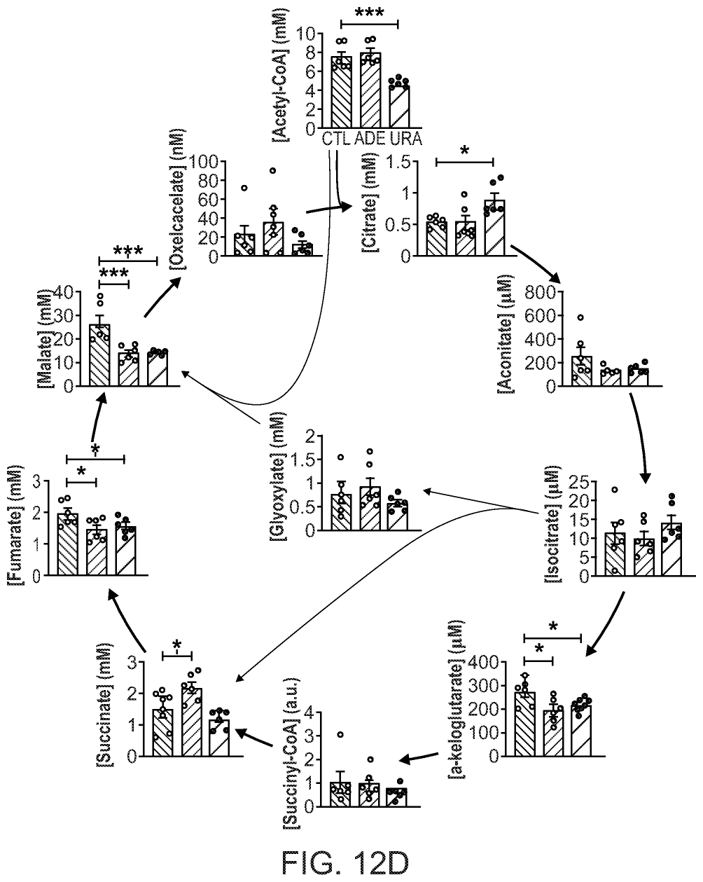

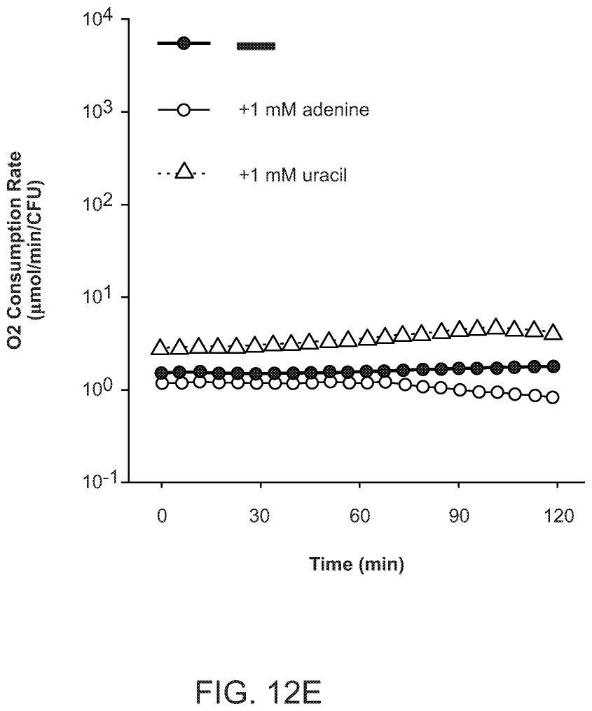

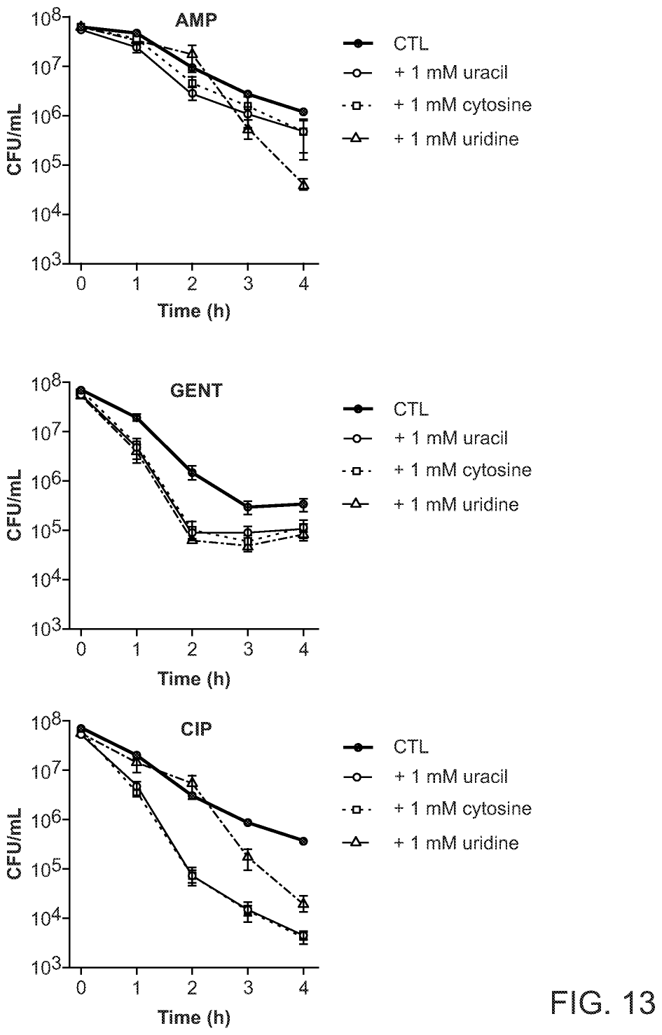

Input-output relationships between E. coli metabolism and antibiotic lethality were systematically quantified by measuring antibiotic IC₅₀s following supplementation with metabolites known to participate in E. coli metabolism (FIG. 2A). To avoid the potentially confounding effects of stationary phase physiology on antibiotic tolerance, experiments were performed using exponentially growing E. coli MG1655 cells. These cells were grown in MOPS defined minimal medium (Neidhardt et al., 1974) and were systematically screened with an unbiased and semi-comprehensive library of metabolites, against AMP, CIP and GENT. Screened metabolites were derived from the Biolog phenotype microarrays (PMs) 1-4 (Bochner, 2009), which are comprised of diverse carbon, nitrogen, phosphorus and sulfur species. These PMs contain 206 unique amino acids, carbohydrates, nucleotides and organic acids that are included in the iJO1366 genome-scale model of E. coli metabolism. Antibiotic responses to these 206 metabolites were used for subsequent analyses (Table 1).

Observed changes in antibiotic IC₅₀s were modest—in most cases, less than two-fold (FIG. 2B and Table 2). Hierarchical clustering of the measured IC₅₀s revealed that the metabolite response profiles differed between AMP, CIP and GENT, highlighting their different biochemical targets. However, several metabolites appeared to commonly potentiate or inhibit efficacy across multiple antibiotics, indicating shared metabolic mechanisms of action. Many nitrogen, phosphorus and sulfur metabolites increased antibiotic IC₅₀s, while many carbon metabolites decreased IC₅₀s, consistent with certain previous observations (Yang et al., 2017b). These raw data indicated that the measured antibiotic lethality responses to metabolite perturbations occurred through specific metabolic pathways, rather than generically as a response to medium enrichment.

### Example 4: Conventional Bioinformatic Analyses do not Provide Novel Mechanistic Insights

To test the capabilities of conventional bioinformatic analyses for yielding mechanistic insights into how the screened metabolites alter antibiotic lethality, an enrichment analysis was first performed on metabolites that elicited a≥2-fold change in IC₅₀—a conventional definition for a screening “hit” (Table 3). For each antibiotic, a metabolite set enrichment analysis was performed in Ecocyc. For AMP (2 metabolites≥2-fold change in IC₅₀) and GENT (8 metabolites≥2-fold change in IC₅₀), no pathways were enriched with less than a 5% false discovery rate (FDR) (q<0.05). For CIP (19 metabolites≥2-fold change in IC₅₀), several non-specific pathways related to protein translation were identified, with top enrichments including ‘aminoacyl-tRNA charging’ (p=1.98e-6), ‘proteinogenic amino acids biosynthesis’ (p=2.50e-6) and ‘amino acids degradation’ (p=1.27e-5) (Table 4). These findings were consistent with previous observations that protein translation inhibitors generally exert antagonistic effects on antibiotic lethality (Lobritz et al., 2015; Ocampo et al., 2014). Collectively, these results illustrated two common weaknesses in conventional bioinformatic approaches for analyzing biochemical screens: statistical power limitations and low specificity associations.

### Example 5: White-Box Machine Learning Revealed Known and New Antibiotic Mechanisms of Action

The white-box machine learning approach was applied and metabolic network states corresponding to supplementation with each metabolite used in the screen were prospectively modelled. For each metabolite, metabolic states were simulated by first adding exchange reactions to the E. coli metabolic model, which enabled uptake of each metabolite from the extracellular environment. Parsimonious flux balance analysis (pFBA) (Lewis et al., 2010) was then performed in conditions simulating MOPS minimal medium and optimized for the biomass objective function (Table 5). Although this approach does not explicitly model contributions by gene expression towards changes in metabolism, benchmarking studies have demonstrated that principles of growth maximization and parsimony are sufficient for accurately predicting metabolism in defined metabolic environments (Machado and Herrgard, 2014).

For each antibiotic, metabolic pathway mechanisms were identified by first conducting a dimension-reducing machine learning regression task, and then performing hypergeometric statistical testing on metabolic reactions comprising the outputted predictive model using pathway-reaction sets curated by Ecocyc. The measured changes in antibiotic IC₅₀ were jointly learned on the set of simulated metabolic network states using multitask elastic net (Carana, 1997; Zou and Hastie, 2005), yielding 477 reactions predicted to alter antibiotic lethality. For each antibiotic, reactions with coefficients whose magnitude were less than or equal to half the standard deviation of all coefficients were removed to exclude spurious reactions selected by joint learning. For AMP, CIP and GENT, this yielded 189, 208 and 204 reactions, respectively (Table 6). Next, hypergeometric statistics were performed on Ecocyc-curated pathways. Of the 431 metabolic pathways curated by Ecocyc, only 13 were found to be statistically significant with less than 5% FDR for at least one antibiotic (Table 7).

Because the current white-box machine learning approach yields pathway mechanisms, the relative contributions of each metabolic pathway to the lethal mechanisms of each antibiotic could be quantified. Pathway scores were computed for each pathway and antibiotic by performing least squares regression on the changes in antibiotic IC₅₀ and then log-transforming the average non-zero regression coefficients for all reactions in each pathway. Identified pathways primarily clustered into three groups, based on their pathway scores (FIG. 3). One cluster possessed central carbon metabolism pathways (‘Superpathway of glycolysis, pyruvate dehydrogenase, TCA, and glyoxylate bypass’; ‘Superpathway of glyoxylate bypass and TCA’; ‘TCA Cycle I (prokaryotic)’) with similar pathway directionality for AMP, CIP and GENT (indicated by the sign of the pathway score). These findings were consistent with several prior studies which demonstrated the TCA cycle to be a shared mechanism in antibiotic lethality (Kohanski et al., 2007; Meylan et al., 2017; Nandakumar et al., 2014) and validated the fidelity of the above-mentioned white-box machine learning approach.

Remarkably, a second cluster appeared possessing purine biosynthesis pathways (‘Superpathway of histidine, purine, and pyrimidine biosynthesis’; ‘Superpathway of purine nucleotides de novo biosynthesis II’) with shared directionality between AMP and CIP, and opposite directionality for GENT. Prior to the instant disclosure, purine biosynthesis does not appear to have been implicated as a mechanism of antibiotic lethality in any previous biochemical or chemogenomic screen. To better understand these differences in pathway directionality, regression coefficients for each reaction were examined and a corresponding reaction score was computed by log-transforming the reaction magnitudes. These analyses identified early steps in the purine biosynthesis pathway as being primarily responsible for the predicted differences for AMP and CIP from GENT (FIG. 8). These findings illustrated how white-box machine learning can reveal new mechanisms of action with high biochemical specificity.

### Example 6: Purine Biosynthesis Activity Participated in Antibiotic Lethality

Motivated by the above model-guided machine learning predictions, whether perturbations to purine biosynthesis would alter antibiotic lethality was examined. From the predictions, it appeared likely that genetic deletion of enzymes involved in purine metabolism would exert differential effects on AMP and CIP lethality, as compared to GENT lethality. Indeed, E. coli mutants deficient for purD (glycinamide ribonucleotide synthetase), purE (N⁵-carboxyaminoimidazole ribonucleotide mutase), purK (5-(carboxyamino)imidazole ribonucleotide synthase), or purM (phosphoribosylformylglycinamide cyclo-ligase), early steps in purine biosynthesis (FIG. 4A), exhibited significant decreases in AMP and CIP lethality, but increased GENT lethality, as compared to wildtype (FIG. 4B). Similarly, biochemical inhibition of purine biosynthesis with 6-mercaptopurine, a PurF (amidophosphoribosyltransferase) inhibitor, decreased AMP and CIP lethality, but increased GENT lethality (FIG. 4C). These effects appeared to be specific to purine metabolism, as genetic deletion of enzymes involved in pyrimidine biosynthesis did not elicit significant differences in AMP, CIP or GENT lethality (FIG. 9A).

Cells deficient for glyA (serine hydroxymethyltransferase), which participates in producing tetrahydrofolate co-factors through the folate cycle, also exhibited decreased AMP and CIP lethality, but increased GENT lethality (FIG. 4D). Similar phenotypes were observed under combination treatment with trimethoprim, a potent biochemical inhibitor of FolA (dihydrofolate reductase; FIG. 9B), consistent with previous findings (Lobritz et al., 2015; Ocampo et al., 2014: Paisley and Washington, 1978).

The instant results also indicated that stimulation of purine biosynthesis would likely elicit opposite effects on antibiotic lethality than the above-described inhibition achieved by genetic and biochemical perturbations. Indeed, biochemical supplementation with the purine biosynthesis substrates phosphoribosyl pyrophosphate (prpp) and glutamine (gln) (FIG. 4A, blue) led to increased AMP and CIP lethality, and decreased GENT lethality (FIG. 4E). Collectively, these data supported the model-driven hypothesis that purine biosynthesis participates in antibiotic lethality and demonstrate how model-guided machine learning can provide reductive, hypothesis-driven mechanistic insights into drug efficacy.

### Example 7: Adenine Limitation Contributed to Antibiotic Lethality

Bactericidal antibiotics significantly alter bacterial metabolism as part of their lethality, increasing the abundance of intracellular central carbon metabolites and disrupting the nucleotide pool (Belenky et al., 2015; Nandakumar et al., 2014; Zampieri et al., 2017). Nucleotide pool disruptions include rapid depletion of free intracellular adenine, guanine and cytosine, and marked accumulation of intracellular uracil (FIG. 10). Additionally, nucleotide biosynthesis pathways auto-regulated with internal feedback inhibition biochemically driven by their nucleotide end-products (FIG. 5A; Lehninger et al., 2013). Based on the predictions from the above-described white-box machine learning approach and the above observations, it appeared likely that purine supplementation could rescue antibiotic-induced purine depletion, and consequently decrease the demand for purine biosynthesis, thereby reducing antibiotic lethality. Of note, supplementation with adenine (FIG. 5B, red), but not guanine, decreased antibiotic lethality in wildtype cells. Without wishing to be bound by theory, these results indicated that adenine limitation rather than guanine limitation drives purine biosynthesis activity under antibiotic stress. Results of the instant studies also indicated that pyrimidine supplementation would inhibit pyrimidine biosynthesis and promote purine biosynthesis activity via prpp accumulation, and consequently increase antibiotic lethality. Indeed, supplementation with uracil or cytosine potentiated antibiotic lethality (FIG. 5C, blue). Collectively, these data indicated that purine biosynthesis likely participates in antibiotic lethality and further indicates that antibiotic-induced purine biosynthesis is likely driven by adenine limitation.

### Example 8: Adenine Supplementation Reduced ATP Demand and Central Carbon Metabolism Activity

Purine biosynthesis is energetically expensive, costing eight ATP molecules to synthesize one adenine molecule from one glucose molecule (Lehninger et al., 2013). To better understand the mechanistic basis for the observed differences in antibiotic lethality under adenine or uracil supplementation, the simulated metabolic network states corresponding to these perturbations were examined (Table 5). Model simulations predicted that adenine supplementation would decrease purine biosynthesis and consequently decrease ATP utilization by nucleotide synthesis and salvage reactions, while uracil supplementation would not (FIG. 6A). Model simulations also predicted that as a result of these changes, overall flux through central carbon metabolism pathways would decrease, reducing the activity of enzymes involved in cellular respiration and oxidative phosphorylation, such as succinate dehydrogenase (FIG. 11). These modeling results were consistent with previous observations that glycolytic flux is controlled by ATP demand (Koebmann et al., 2002).

These metabolic modeling predictions were tested by quantifying the intracellular concentrations of central carbon metabolism and energy currency metabolites from E. coli cells grown in MOPS minimal medium and supplemented with either adenine or uracil (FIG. 6B; Table 8). Under these conditions, cell growth did not significantly change within the first hour of supplementation (FIG. 12A), but intracellular adenine nucleotides did accumulate under exogenous adenine addition (FIG. 12B). Consistent with model predictions that adenine supplementation would inhibit succinate dehydrogenase activity, intracellular succinate increased, while intracellular fumarate decreased (FIG. 6C). Model simulations additionally predicted that ATP synthesis would decrease under adenine supplementation (FIG. 6D, left). Consistent with this, a modest decrease in the adenylate energy charge, an index for the energy state of a cell (Chapman and Atkinson, 1977), was observed (FIG. 6D, right). The relative changes in intracellular nicotinamide adenine dinucleotides under adenine or uracil supplementation were examined (FIG. 12C) and a modest decrease in the NADPH/NADP⁺ ratio, but not the NADH/NAD⁺ ratio, following exogenous adenine addition (FIG. 6E) was observed. Together, these results support the model predictions that adenine supplementation decreases central carbon metabolism activity (decreased adenylate energy charge) and cell anabolism (decreased NADPH/NADP⁺ ratio) without significantly changing cell catabolism (unchanged NADH/NAD⁺ ratio) (FIG. 12D) (Andersen and von Meyenburg, 1977; Chapman and Atkinson, 1977).

The metabolic modeling simulations further predicted that decreases in oxidative phosphorylation under adenine supplementation lead to decreases in cellular oxygen consumption (FIG. 6F, left). These modeling predictions were tested using a Seahorse XF Analyzer and measured changes in the oxygen consumption rate (OCR) following antibiotic treatment, with or without adenine or uracil supplementation. Antibiotic treatment with AMP, CIP or GENT increased cellular OCR (FIG. 6F, black), in contrast to control conditions (FIG. 12E), supporting previous observations that cellular respiration is important for antibiotic lethality (Gutierrez et al., 2017; Lobritz et al., 2015). Importantly, adenine supplementation significantly repressed cellular OCR under antibiotic treatment (FIG. 6F, red), consistent with model predictions, while uracil enhanced cellular OCR (FIG. 6F, blue). These results directly supported the hypothesis that central carbon metabolism activity and cellular respiration are increased under antibiotic stress to satisfy the elevated ATP demand resulting from purine biosynthesis. Collectively, the data and simulations indicate that adenine limitation resulting from antibiotic treatment drives purine biosynthesis, which increases ATP demand, fueling the redox-associated metabolic alterations involved in antibiotic lethality (Dwyer et al., 2014; FIG. 7).

### Example 9: Treatment of a Subject Having COVID-19

A subject having COVID-19 is administered a pharmaceutical composition containing a pyrimidine and an antiviral agent and/or an antimicrobial agent. In one exemplary embodiment, the pyrimidine is administered together with remdesivir, by intravenous infusion. Alternatively, the pyrimidine is administered together with a quinolone, orally.

Additionally or alternatively, the subject having COVID-19 is administered a pharmaceutical composition containing a pyrimidine, together with one or more of the following therapies: an antibiotic for treatment of bacterial pneumonia; a cough medicine, and/or a fever reducer/pain reliever (e.g., aspirin, ibuprofen, acetaminophen).

A COVID-19-related lung infection (e.g., a pneumonia, optionally a viral pneumonia) is assessed for amelioration in the subject after administration of the pyrimidine and the antiviral agent and/or antimicrobial agent to the subject.

### Tables

Table 1. Metabolite mappings from Biolog PMs 1-4 to iJO1366 E. coli model: related to FIGS. 2A and 2B.
See U.S. Ser. No. 62/845,053.
Table 2. Measured antibiotic IC₅₀s from metabolite screens on Biolog PMs 1-4. Data are computed from fitting logistic functions to 4 h OD₆₀₀ measurements from n≥3 biological replicates; related to FIGS. 2A and 2B.
See U.S. Ser. No. 62/845,053.
Table 3. Metabolites eliciting a ≥2-fold change in IC50 for each antibiotic; related to FIGS. 2A and 2B.
See U.S. Ser. No. 62/845,053.
Table 4. Metabolic pathways enriched from metabolites that elicited ≥2-fold change in ciprofloxacin (CIP) IC₅₀, identified by Metabolite Set Enrichment Analysis in Ecocyc; related to FIGS. 2A and 2B.
See U.S. Ser. No. 62/845,053.
Table 5. Simulated metabolic flux states associated with each metabolite screening condition; related to FIG. 3.
See U.S. Ser. No. 62/845,053.
Table 6. Metabolic reactions identified by multitask elastic net regularization to be enriched for in antibiotic lethality; related to FIG. 3.
See U.S. Ser. No. 62/845,053.
Table 7. Metabolic pathways predicted to participate in ampicillin (AMP), ciprofloxacin (CIP) or gentamicin (GENT) lethality; related to FIG. 3.
See U.S. Ser. No. 62/845,053.
Table 8. Measured intracellular metabolite concentrations under adenine or uracil supplementation. Data are reported as mean±SD and computed from LC-MS/MS measurements from n=3 biological replicates; related to FIGS. 6A-6F.
See U.S. Ser. No. 62/845,053.

TABLE 9
Oligonucleotides used for verifying E. coli gene
deletions; related to STAR Methods.

| | SEQ ID |
|---|---|
| Oligonucleotides | NO: |
| glyA forward primer: AGAAATCCGTTTCCGGTTGC | 1 |
| glyA reverse primer: CCCAGAACGGTATCACCTGG | 2 |
| purD forward primer: TATTGCGATGCTCTTCACCGA | 3 |
| purD reverse primer: CGTTTGTGATCCTGGCTGGT | 4 |
| purE forward primer: GAGAGTTGTGCACCACAGGA | 5 |
| purE reverse primer: CAGCACTTCGTCGGTCTGG | 6 |
| purK forward primer: CAGTTACTTGCCGAACGCAG | 7 |
| purK reverse primer: TTTTGTGTCCAGTGACCGCT | 8 |
| purM forward primer: ATTGACGCGGGTAATGCTCT | 9 |
| purM reverse primer: CCGCGACATCGTAATCCTCA | 10 |
| pyrC forward primer: TGGTGCATGGTGAAGTGACA | 11 |
| pyrC reverse primer: CCAGCGTGTCATCAGTCAGT | 12 |
| pyrE forward primer: GGTGGATTCCGGCATTGAGT | 13 |
| pyrE reverse primer: TGCGGCACAGTTGCAGTAAT | 14 |
| Keio insert primer: GATGTTTCGCTTGGTGGTCG | 15 |

### REFERENCES

Adolfsen, K. J., and Brynildsen, M. P. (2015). Futile cycling increases sensitivity toward oxidative stress in Escherichia coli. Metab Eng 29, 26-35.
Allison, K. R., Brynildsen, M. P., and Collins, J. J. (2011). Metabolite-enabled eradication of bacterial persisters by aminoglycosides. Nature 473, 216-220.
Andersen, K. B., and von Meyenburg, K. (1977). Charges of nicotinamide adenine nucleotides and adenylate energy charge as regulatory parameters of the metabolism in Escherichia coli. J Biol Chem 252, 4151-4156.
Baba, T., Am, T., Hasegawa. M., Takai, Y., Okumura, Y., Baba, M., Datsenko, K. A., Tomita, M., Wanner, B. L., and Mori, H. (2006). Construction of Escherichia coli K-12 in-frame, single-gene knockout mutants: the Keio collection. Mol Syst Biol 2, 2006 0008.
Babin, B. M., Atangcho, L., van Eldijk, M. B., Sweredoski, M. J., Moradian, A., Hess, S., Tolker-Nielsen, T., Newman, D. K., and Tirrell, D. A. (2017). Selective Proteomic Analysis of Antibiotic-Tolerant Cellular Subpopulations in Pseudomonas aeruginosa Biofilms. MBio 8.
Bald, D., Villellas, C., Lu, P., and Koul, A. (2017). Targeting Energy Metabolism in Mycobacterium tuberculosis, a New Paradigm in Antimycobacterial Drug Discovery. MBio 8.
Basan, M., Hui, S., Okano, H., Zhang, Z., Shen, Y., Williamson, J. R., and Hwa, T. (2015). Overflow metabolism in Escherichia coli results from efficient proteome allocation. Nature 528, 99-104.
Belenky, P., Ye, J. D., Porter, C. B., Cohen, N. R., Lobritz, M. A., Ferrante, T., Jain, S., Korry, B. J., Schwarz, E. G., Walker, G. C., et al. (2015). Bactericidal Antibiotics Induce Toxic Metabolic Perturbations that Lead to Cellular Damage. Cell Rep 13, 968-980.
Bochner, B. R. (2009). Global phenotypic characterization of bacteria. FEMS Microbiol Rev 33, 191-205. Bottou, L. (2014). From machine learning to machine reasoning. Machine Learning 94, 133-149. Brown, E. D., and Wright, G. D. (2016). Antibacterial drug discovery in the resistance era. Nature 529, 336-343.
Brunk, E., Sahoo, S., Zielinski, D. C., Altunkaya, A., Drager, A., Mih, N., Gatto, F., Nilsson, A., Preciat Gonzalez, G. A., Aurich, M. K., et al. (2018). Recon3D enables a three-dimensional view of gene variation in human metabolism. Nat Biotechnol 36, 272-281.
Bycroft, C., Freeman, C., Petkova, D., Band, G., Elliott, L. T., Sharp, K., Motyer, A., Vukcevic, D., Delaneau, O., O'Connell, J., et al. (2018). The UK Biobank resource with deep phenotyping and genomic data. Nature 562, 203-209.
Camacho, D. M., Collins, K. M., Powers. R. K., Costello, J. C., and Collins, J. J. (2018). Next-Generation Machine Learning for Biological Networks. Cell 173, 1581-1592.
Carrera, J., and Covert, M. W. (2015). Why Build Whole-Cell Models? Trends Cell Biol 25, 719-722. Caruana, R. (1997). Multitask Learning. Machine Learning 28, 41-75.
Cekic, C., and Linden, J. (2016). Purinergic regulation of the immune system. Nat Rev Immunol 16, 177-192.
Certain, L. K., Way, J. C., Pezone, M J, and Collins, J. J. (2017). Using Engineered Bacteria to Characterize Infection Dynamics and Antibiotic Effects In Vivo. Cell Host Microbe 22, 263-268 e264. Chapman, A. G., and Atkinson, D. E. (1977). Adenine nucleotide concentrations and turnover rates. Their correlation with biological activity in bacteria and yeast. Adv Microb Physiol 15, 253-306.
Ching, T., Himmelstein, D. S., Beaulieu-Jones, B. K., Kalinin, A. A., Do, B. T., Way, G. P., Ferrero, E., Agapow, P. M., Zietz, M., Hoffman, M. M., et al. (2018). Opportunities and obstacles for deep learning in biology and medicine. J R Soc Interface 15.
Cho, H., Uehara, T., and Bernhardt, T. G. (2014). Beta-lactam antibiotics induce a lethal malfunctioning of the bacterial cell wall synthesis machinery. Cell 159, 1300-1311.
Dunphy. L. J., and Papin, J. A. (2017). Biomedical applications of genome-scale metabolic network reconstructions of human pathogens. Curr Opin Biotechnol 51, 70-79.
Dwyer, D. J., Belenky, P. A., Yang, J. H., MacDonald, I. C., Martell, J. D., Takahashi, N., Chan, C. T., Lobritz, M. A., Braff, D., Schwarz, E. G., et al. (2014). Antibiotics induce redox-related physiological alterations as part of their lethality. Proc Natl Acad Sci USA 111, E2100-2109.
Dwyer, D. J., Collins, J. J., and Walker, G. C. (2015). Unraveling the physiological complexities of antibiotic lethality. Annu Rev Pharmacol Toxicol 55, 313-332.
El Zahed, S. S., and Brown, E. D. (2018). Chemical-Chemical Combinations Map Uncharted Interactions in Escherichia coli under Nutrient Stress. iScience 2, 168-181.
Fan, X. Y., Tang, B. K., Xu, Y. Y., Han, A. X., Shi, K. X., Wu, Y. K., Ye, Y., Wei, M. L., Niu, C., Wong, K. W., et al. (2018). Oxidation of dCTP contributes to antibiotic lethality in stationary-phase mycobacteria. Proc Natl Acad Sci USA 115, 2210-2215.
Foti, J. J., Devadoss, B., Winkler, J. A., Collins, J. J., and Walker, G. C. (2012). Oxidation of the guanine nucleotide pool underlies cell death by bactericidal antibiotics. Science 336, 315-319.
French, S., Coutts, B. E., and Brown, E. D. (2018). Open-Source High-Throughput Phenomics of Bacterial Promoter-Reporter Strains. Cell Syst.
French, S., Mangat, C., Bharat, A., Cote, J. P., Mori, H., and Brown, E. D. (2016). A robust platform for chemical genomics in bacterial systems. Mol Biol Cell 27, 1015-1025.
Gil, Y., Greaves, M., Hendler, J., and Hirsh, H. (2014). Artificial Intelligence. Amplify scientific discovery with artificial intelligence. Science 346, 171-172.
Gruber, C. C., and Walker. G. C. (2018). Incomplete base excision repair contributes to cell death from antibiotics and other stresses. DNA Repair.
Gutierrez, A., Jain, S., Bhargava, P., Hamblin, M., Lobritz, M. A., and Collins, J. J. (2017). Understanding and Sensitizing Density-Dependent Persistence to Quinolone Antibiotics. Mol Cell 68, 1147-1154 e1143. Holm, A. K., Blank, L. M., Oldiges, M., Schmid, A., Solem, C., Jensen, P. R., and Vemuri, G. N. (2010). Metabolic and transcriptional response to cofactor perturbations in Escherichia coli. J Biol Chem 285, 17498-17506.
Honaker, J., King, G., and Blackwell, M. (2011). Amelia II: A Program for Missing Data. J Stat Softw 45, 1-47.
Hui, S., Silverman, J. M., Chen, S. S., Erickson, D. W., Basan, M., Wang, J., Hwa, T., and Williamson, J. R. (2015). Quantitative proteomic analysis reveals a simple strategy of global resource allocation in bacteria. Mol Syst Biol 11, 784.
Ideker, T., Galitski, T., and Hood, L. (2001). A new approach to decoding life: systems biology. Annu Rev Genomics Hum Genet 2, 343-372.
Kanarek, N., Keys, H. R., Cantor, J. R., Lewis, C. A., Chan, S. H., Kunchok, T., Abu-Remaileh, M., Freinkman, E., Schweitzer, L. D., and Sabatini, D. M. (2018). Histidine catabolism is a major determinant of methotrexate sensitivity. Nature 559, 632-636.
Karr, J. R., Sanghvi, J. C., Macklin, D. N., Gutschow, M. V., Jacobs, J. M., Bolival, B., Jr., Assad-Garcia, N., Glass, J. I., and Covert, M. W. (2012). A whole-cell computational model predicts phenotype from genotype. Cell 150, 389-401.
Keenan, A. B., Jenkins, S. L., Jagodnik, K. M., Koplev, S., He, E., Torre, D., Wang, Z., Dohlman, A. B., Silverstein, M. C., Lachmann. A., et al. (2018). The Library of Integrated Network-Based Cellular Signatures NIH Program: System-Level Cataloging of Human Cells Response to Perturbations. Cell Syst 6, 13-24.
Keseler, I. M., Mackie, A., Santos-Zavaleta, A., Billington, R., Bonavides-Martinez, C., Caspi, R., Fulcher, C., Gama-Castro, S., Kothari, A., Krummenacker, M., et al. (2017). The EcoCyc database: reflecting new knowledge about Escherichia coli K-12. Nucleic Acids Res 45, D543-D550.
Koebmann, B. J., Westerhoff, H. V., Snoep, J. L., Nilsson, D., and Jensen, P. R. (2002). The glycolytic flux in Escherichia coli is controlled by the demand for ATP. J Bacteriol 184, 3909-3916.
Kohanski, M. A., Dwyer, D. J., and Collins, J. J. (2010). How antibiotics kill bacteria: from targets to networks. Nat Rev Microbiol 8, 423-435.
Kohanski, M. A., Dwyer, D. J., Hayete, B., Lawrence, C. A., and Collins, J. J. (2007). A common mechanism of cellular death induced by bactericidal antibiotics. Cell 130, 797-810.
Lee, H. H., and Collins, J. J. (2011). Microbial environments confound antibiotic efficacy. Nat Chem Biol 8, 6-9.
Lehninger, A. L., Nelson, D. L., and Cox, M. M. (2013). Lehninger principles of biochemistry, 6th edn (New York: W. H. Freeman).
Lewis, N. E., Hixson, K. K., Conrad, T. M., Lerman, J. A., Charusanti, P., Polpitiya, A. D., Adkins, J. N., Schramm, G., Purvine, S. O., Lopez-Ferrer, D., et al. (2010). Omic data from evolved E. coli are consistent with computed optimal growth from genome-scale models. Mol Syst Biol 6, 390.
Litichevskiy, L., Peckner, R., Abelin, J. G., Asiedu, J. K., Creech, A. L., Davis, J. F., Davison, D., Dunning, C. M., Egertson, J. D., Egri, S., et al. (2018). A Library of Phosphoproteomic and Chromatin Signatures for Characterizing Cellular Responses to Drug Perturbations. Cell Syst 6, 424-443 c427.
Lobritz, M. A., Belenky, P., Porter, C. B., Gutierrez, A., Yang, J. H., Schwarz, E. G., Dwyer, D. J., Khalil, A. S., and Collins, J. J. (2015). Antibiotic efficacy is linked to bacterial cellular respiration. Proc Natl Acad Sci USA 112, 8173-8180.
Lu, R., Lee, G. C., Shultz, M., Dardick, C., Jung, K., Phetsom, J., Jia, Y., Rice, R. H., Goldberg, Z., Schnable, P. S., et al. (2008). Assessing probe-specific dye and slide biases in two-color microarray data. BMC Bioinformatics 9, 314.
Ma, J., Yu, M. K., Fong, S., Ono, K., Sage, E., Demchak, B., Sharan, R, and Ideker, T. (2018). Using deep learning to model the hierarchical structure and function of a cell. Nat Methods 15, 290-298. Machado, D., and Herrgard, M. (2014). Systematic evaluation of methods for integration of transcriptomic data into constraint-based models of metabolism. PLoS Comput Biol 10, e1003580. Mack, S. G., Turner, R. L., and Dwyer. D. J. (2018). Achieving a Predictive Understanding of Antimicrobial Stress Physiology through Systems Biology. Trends Microbiol 26, 296-312.
McCloskey, D., Gangoiti, J. A., King, Z. A., Naviaux, R. K., Barshop, B. A., Palsson, B. O., and Feist, A. M. (2014). A model-driven quantitative metabolomics analysis of aerobic and anaerobic metabolism in E. coli K-12 MG1655 that is biochemically and thermodynamically consistent. Biotechnol Bioeng 111, 803-815.
McCloskey, D., Xu, J., Schrubbers, L., Christensen. H. B., and Herrgard. M. J. (2018). RapidRIP quantifies the intracellular metabolome of 7 industrial strains of E. coli. Metab Eng 47, 383-392.
Megchelenbrink, W., Huynen. M., and Marchiori, E. (2014). optGpSampler: an improved tool for uniformly sampling the solution-space of genome-scale metabolic networks. PLoS One 9, e86587. Meylan, S., Porter, C. B., Yang, J. H., Belenky, P., Gutierrez. A., Lobritz, M. A., Park, J., Kim, S. H., Moskowitz, S. M., and Collins. J. J. (2017). Carbon Sources Tune Antibiotic Susceptibility in Pseudomonas aeruginosa via Tricarboxylic Acid Cycle Control. Cell Chem Biol 24, 195-206.
Milo, R., and Phillips, R. (2016). Cell biology by the numbers (New York, N.Y.: Garland Science, Taylor & Francis Group).
Monk, J. M., Lloyd, C. J., Brunk, E., Mih, N., Sastry, A., King, Z., Takeuchi, R., Nomura, W., Zhang, Z., Mori, H., et al. (2017). iML1515, a knowledgebase that computes Escherichia coli traits. Nat Biotechnol 35, 904-908.
Murima, P., McKinney, J. D., and Pethe, K. (2014). Targeting bacterial central metabolism for drug development. Chem Biol 21, 1423-1432.
Nandakumar, M., Nathan, C., and Rhee, K. Y. (2014). Isocitrate lyase mediates broad antibiotic tolerance in Mycobacterium tuberculosis. Nat Commun 5, 4306.
Neidhardt, F. C., Bloch, P. L., and Smith, D. F. (1974). Culture medium for enterobacteria. J Bacteriol 119, 736-747.
Oberhardt, M. A., Yizhak, K., and Ruppin, E. (2013). Metabolically re-modeling the drug pipeline. Curr Opin Pharmacol 13, 778-785.
Ocampo, P. S., Lazar. V., Papp. B., Amoldini, M., Abel zur Wiesch, P., Busa-Fekete, R., Fekete, G., Pal, C., Ackermann, M., and Bonhoeffer, S. (2014). Antagonism between bacteriostatic and bactericidal antibiotics is prevalent. Antimicrob Agents Chemother 58, 4573-4582.
Orth, J. D., Conrad, T. M., Na, J., Lennan, J. A., Nam, H., Feist, A. M., and Palsson, B. O. (2011). A comprehensive genome-scale reconstruction of Escherichia coli metabolism-2011. Mol Syst Biol 7, 535. Paisley, J. W., and Washington, J. A., 2nd (1978). Synergistic activity of gentamicin with trimethoprim or sulfamethoxazole-trimethoprim against Escherichia coli and Klebsiella pneumoniae. Antimicrob Agents Chemother 14, 656-658.
Pedregosa, F., Varoquaux, G., Gramfort, A., Michel, V., Thirion, B., Grisel, O., Blondel, M., Prettenhofer, P., Weiss, R., Dubourg, V., et al. (2011). Scikit-learn: Machine Learning in Python. J Mach Learn Res 12, 2825-2830.
Roses. A. D. (2008). Pharmacogenetics in drug discovery and development: a translational perspective. Nat Rev Drug Discov 7, 807-817.
Schellenberger, J., Que, R., Fleming, R. M., Thiele, I., Orth, J. D., Feist, A. M., Zielinski, D. C., Bordbar, A., Lewis, N. E., Rahmanian, S., et al. (2011). Quantitative prediction of cellular metabolism with constraint-based models: the COBRA Toolbox v2.0. Nat Protoc 6, 1290-1307.
Scott, M., Klumpp, S., Mateescu, E. M., and Hwa, T. (2014). Emergence of robust growth laws from optimal regulation of ribosome synthesis. Mol Syst Biol 10, 747.
Serpi, M., Ferrari, V., and Pertusati, F. (2016). Nucleoside Derived Antibiotics to Fight Microbial Drug Resistance: New Utilities for an Established Class of Drugs? J Med Chem 59, 10343-10382.
Shan, Y., Brown Gandt, A., Rowe, S. E., Deisinger, J. P., Conlon, B. P., and Lewis, K. (2017). ATP-Dependent Persister Formation in Escherichia coli. MBio 8.
Shetty, A., and Dick, T. (2018). Mycobacterial Cell Wall Synthesis Inhibitors Cause Lethal ATP Burst. Front Microbiol 9, 1898.
Stark, C., Breitkreutz, B. J., Reguly, T., Boucher, L., Breitkreutz, A., and Tyers, M. (2006). BioGRID: a general repository for interaction datasets. Nucleic Acids Res 34, D535-539.
Topol, E. J. (2019). High-performance medicine: the convergence of human and artificial intelligence. Nat Med 25, 44-56.
Tyers, M., and Wright, G. D. (2019). Drug combinations: a strategy to extend the life of antibiotics in the 21st century. Nat Rev Microbiol 17, 141-155.
Vander Heiden, M. G., and DeBerardinis, R. J. (2017). Understanding the Intersections between Metabolism and Cancer Biology. Cell 168, 657-669.
Wainberg, M., Merico, D., Delong, A., and Frey, B. J. (2018). Deep learning in biomedicine. Nat Biotechnol 36, 829-838.
Wang, T., Wei, J. J., Sabatini, D. M., and Lander, E. S. (2014). Genetic screens in human cells using the CRISPR-Cas9 system. Science 343, 80-84.
Webb, S. (2018). Deep learning for biology. Nature 554, 555-557.
Xie, L., Draizen, E. J., and Boume, P. E. (2017). Harnessing Big Data for Systems Pharmacology. Annu Rev Pharmacol Toxicol 57, 245-262.
Yang, J. H., Bening, S. C., and Collins, J. J. (2017a). Antibiotic efficacy-context matters. Curr Opin Microbiol 39, 73-80.
Yang, J. H., Bhargava, P., McCloskey, D., Mao, N., Palsson, B. O., and Collins, J. J. (2017b). Antibiotic-Induced Changes to the Host Metabolic Environment Inhibit Drug Efficacy and Alter Immune Function. Cell Host Microbe 22, 757-765 e753.
Yang, L., Yurkovich, J. T., King, Z. A., and Palsson, B. O. (2018). Modeling the multi-scale mechanisms of macromolecular resource allocation. Curr Opin Microbiol 45, 8-15.
Yu, K.-H., Beam, A. L., and Kohane, I. S. (2018a). Artificial intelligence in healthcare. Nature Biomedical Engineering 2, 719-731.
Yu, M. K., Ma, J., Fisher, J., Kreisberg, J. F., Raphael, B. J., and Ideker, T. (2018b). Visible Machine Learning for Biomedicine. Cell 173, 1562-1565.
Yuan, H., Paskov, I., Paskov, H., Gonzalez, A. J., and Leslie, C. S. (2016). Multitask learning improves prediction of cancer drug sensitivity. Sci Rep 6, 31619.
Zampieri, M., Zimmermann, M., Claassen, M., and Sauer, U. (2017). Nontargeted Metabolomics Reveals the Multilevel Response to Antibiotic Perturbations. Cell Rep 19, 1214-1228.
Zhao, X., and Drlica, K. (2014). Reactive oxygen species and the bacterial response to lethal stress. Curr Opin Microbiol 21, 1-6.
Zou, H., and Hastie, T. (2005). Regularization and variable selection via the elastic net. Journal of the Royal Statistical Society: Series B (Statistical Methodology) 67, 301-320.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A pharmaceutical composition comprising:
(a) a pyrimidine; and
(b) an antimicrobial agent and/or an antiviral agent for non-chemotherapeutic use, or a pharmaceutically acceptable salt thereof,
and a pharmaceutically acceptable carrier.

2. The pharmaceutical composition of claim 1, wherein the pyrimidine is selected from the group consisting of uracil, uridine, thymine, thymidine, cytosine and cytidine.

3. The pharmaceutical composition of claim 1, wherein the antimicrobial agent is bactericidal.

4. The pharmaceutical composition of claim 1, wherein the antimicrobial agent is an antibiotic or an antimicrobial peptide.

5. The pharmaceutical composition of claim 4, wherein the antibiotic is selected from the group consisting of a β-lactam antibiotic, an aminoglycoside antibiotic a quinolone antibiotic, a rifamycin (e.g., rifampicin), nitrofurantoin, metronidazole, trimethoprim, a sulfonamide (e.g., sulfamethoxazole), and a salt, analog or derivative thereof.

6. The pharmaceutical composition of claim 5, wherein the β-lactam antibiotic is selected from the group consisting of:
a penicillin derivative (e.g., Benzathine penicillin (benzathine & benzylpenicillin), Benzylpenicillin (penicillin G), Phenoxymethylpenicillin (penicillin V), Procaine penicillin (procaine & benzylpenicillin), Pheneticillin, Cloxacillin, Dicloxacillin, Flucloxacillin, Methicillin, Nafcillin, Oxacillin, Temocillin, Amoxicillin, Ampicillin, Mecillinam, Carbenicillin, Ticarcillin, Azlocillin, Mezlocillin, and Piperacillin),
a cephalosporin (e.g., Cefazolin, Cephalexin, Cephalosporin C, Cephalothin, Cefaclor, Cefamandole, Cefuroxime, Cefotetan, Cefoxitin, Cefixime, Cefotaxime, Cefpodoxime, Ceftazidime, Ceftriaxone, Cefepime, Cefpirome, and Ceftaroline),
a monobactam (e.g., Aztreonam, Tigemonam, Nocardicin A, and Tabtoxinine β-lactam), and
a carbapenem or penem (e.g., Biapenem, Doripenem, Ertapenem, Faropenem, Imipenem, Meropenem, Panipenem, Razupenem, Tebipenem, and Thienamycin).

7. The pharmaceutical composition of claim 5, wherein the aminoglycoside antibiotic is selected from the group consisting of gentamicin, streptomycin, kanamycin A, tobramycin, neomycin B, neomycin C, framycetin, paromomycin, ribostamycin, amikacin, arbekacin, bekanamycin (kanamycin B), dibekacin, spectinomycin, hygromycin B, paromomycin sulfate, netilmicin, sisomicin, isepamicin, verdamicin, astromicin, neamine, ribostamycin, and paromomycinlividomycin.

8. The pharmaceutical composition of claim 5, wherein the quinolone antibiotic is selected from the group consisting of ciprofloxacin, garenoxacin, gatifloxacin, gemifloxacin, levofloxacin, moxifloxacin, fleroxacin, lomefloxacin, nadifloxacin, norfloxacin, ofloxacin, pefloxacin, rufloxacin, balofloxacin, grepafloxacin, pazufloxacin, sparfloxacin, temafloxacin, clinafloxacin, sitafloxacin, prulifloxacin, besifloxacin, delafloxacin, danofloxacin, difloxacin, enrofloxacin, ibafloxacin, marbofloxacin, orbifloxacin, and sarafloxacin.

9. The pharmaceutical composition of claim 4, wherein the antimicrobial peptide is selected from the group consisting of Bacitracin, Boceprevir, Dalbavancin, Daptomycin, Enfuvirtide, Oritavancin, Teicoplanin, Telaprevir, Telavancin, Vancomycin, and Guavanin 2.

10. The pharmaceutical composition of claim 1, further comprising a β-lactamase inhibitor, optionally wherein the β-lactamase inhibitor is selected from the group consisting of sulbactam, tebipenem, a Boron based transition state inhibitor (e.g., Ec19), clavulanic acid, tazobactam, avibactam and relebactam.

11. The pharmaceutical composition of claim 4, wherein the antibiotic is present in an amount between 0.1 g and 2.0 g.

12. The pharmaceutical composition of claim 1, wherein the antiviral agent is an agent is selected from the group consisting of Abacavir (use for HIV), Acyclovir (Aciclovir—use for herpes e.g. Chicken pox), Adefovir (use for chronic Hepatitis B), Amantadine (use for influenza), Ampligen, Amprenavir (Agenerase—Use for inhibition of HIV), Arbidol, Atazanavir, Atripla (fixed dose drug), Balavir, Baloxavir marboxil (Xofluza), Biktarvy, Boceprevir (Victrelis), Cidofovir, Cobicistat (Tybost), Combivir (fixed dose drug), Daclatasvir (Daklinza), Darunavir, Delavirdine, Descovy, Didanosine, Docosanol, Dolutegravir, Doravirine (Pifeltro), Ecoliever, Edoxudine, Efavirenz, Elvitegravir, Emtricitabine, Enfuvirtide, Entecavir, Etravirine (Intelence), Famciclovir, Fixed dose combination (antiretroviral), Fomivirsen, Fosamprenavir, Foscarnet, Fosfonet, Fusion inhibitor, Ganciclovir (Cytovene), Ibacitabine, Ibalizumab (Trogarzo), Idoxuridine, Imiquimod, Imunovir, Indinavir, Inosine, Integrase inhibitor, Interferon type I, Interferon type IL, Interferon type III, Interferon, Lamivudine, Letermovir (Prevymis), Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Peginterferon alfa-2b, Penciclovir, Peramivir (Rapivab), Pleconaril, Podophyllotoxin, Protease inhibitor (pharmacology), Pyramidine, Raltegravir, Remdesivir, Reverse transcriptase inhibitor, Ribavirin, Rilpivirine (Edurant), Rimantadine, Ritonavir, Saquinavir, Simeprevir (Olysio), Sofosbuvir, Stavudine, Synergistic enhancer (antiretroviral), Telaprevir, Telbivudine (Tyzeka), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine.

13. The pharmaceutical composition of claim 1, wherein the pyrimidine is provided in an amount sufficient to potentiate the rate at which the antimicrobial agent kills a target population of bacteria by at least 10%, as compared to an appropriate control.

14. A method selected from the group consisting of:
A method for sensitizing a bacteria to an antimicrobial agent comprising contacting the bacteria with a pyrimidine, thereby sensitizing the bacteria to the antimicrobial agent;
A method for treating or preventing a bacterial infection in a subject comprising administering a pharmaceutical composition comprising (a) a pyrimidine and (b) an antimicrobial agent and/or an antiviral agent for non-chemotherapeutic use, or a pharmaceutically acceptable salt thereof, and a pharmaceutically acceptable carrier to a subject having or at risk of developing a bacterial infection, thereby treating or preventing the bacterial infection in the subject; and
A method for treating or preventing a viral and/or microbial infection in a subject comprising administering a pharmaceutical composition comprising (a) a pyrimidine and (b) an antimicrobial agent and/or an antiviral agent for non-chemotherapeutic use, or a pharmaceutically acceptable salt thereof, and a pharmaceutically acceptable carrier to a subject having or at risk of developing a viral and/or microbial infection, thereby treating or preventing the viral and/or microbial infection in the subject.

15. The method of claim 14, wherein the bacteria exhibits resistance or tolerance to the antimicrobial agent.

16. The method of claim 14, wherein the bacteria is selected from the group consisting of Escherichia coli, Klebsiella, Staphylococcus, Pseudomonas, Acinetobacter, Enterococcus, Enterobacter and Mycobacteria, optionally wherein the Klebsiella is a Klebsiella pneumoniae, the Staphylococcus is a Staphylococcus aureus, the Pseudomonas is a Pseudomonas aeruginosa, the Acinetobacter is an Acinetobacter baumannii, the Enterococcus is an Enterococcus faecium or an Enterococcus faecalis, or wherein the Mycobacteria is a Mycobacterium smegmatis or a Mycobacterium tuberculosis.

17. The method of claim 14, wherein the pyrimidine is selected from the group consisting of uracil, uridine, thymine, thymidine, cytosine and cytidine.

18. The method of claim 14:
wherein the antimicrobial agent is an antibiotic, optionally wherein the antibiotic is selected from the group consisting of a β-lactam antibiotic, an aminoglycoside antibiotic a quinolone antibiotic, a rifamycin (e.g., rifampicin), nitrofurantoin, metronidazole, trimethoprim, a sulfonamide (e.g., sulfamethoxazole), and a salt, analog or derivative thereof;
wherein the antimicrobial agent is an antimicrobial peptide, optionally wherein the antimicrobial peptide is selected from the group consisting of Bacitracin, Boceprevir, Dalbavancin, Daptomycin, Enfuvirtide, Oritavancin, Teicoplanin, Telaprevir, Telavancin, Vancomycin, and Guavanin 2;
further comprising contacting the bacteria with a β-lactamase inhibitor;
wherein the subject is a mammal;
wherein the subject is human;
wherein the bacterial infection is a bacteremia;
wherein the bacterial infection is an antibiotic resistant or antibiotic tolerant bacterial infection;
wherein the pharmaceutical composition is administered by injection, optionally by intravenous injection;
wherein the bacterial infection is a localized bacterial infection, optionally wherein the localized bacterial infection is a lung infection;
wherein the pharmaceutical composition is administered by aerosolization;
wherein the pharmaceutical composition is administered topically;
wherein the viral and/or microbial infection is a lung infection, optionally a pneumonia, optionally wherein the lung infection is a COVID-19-related lung infection; and/or
wherein the subject has a viral infection, optionally an influenza, rhinovirus and/or coronavirus infection, optionally wherein the subject has a coronavirus infection, optionally wherein the coronavirus infection is COVID-19.

19-29. (canceled)

30. A kit comprising a pyrimidine and an antimicrobial agent and/or an antiviral agent for non-chemotherapeutic use, and instructions for its use.

31. The kit of claim 30, further comprising an agent for measuring phosphoribosyl pyrophosphate accumulation and/or for measuring the lethality of the antimicrobial agent and/or an antiviral agent against a target microbe and/or target virus.

32-35. (canceled)


SEQUENCE LISTING (15 sequences)

SEQ ID NO: 1 - DNA, 20 bp, Artificial
  Synthetic
agaaatccgtttccggttgc

SEQ ID NO: 2 - DNA, 20 bp, Artificial
  Synthetic
cccagaacggtatcacctgg

SEQ ID NO: 3 - DNA, 21 bp, Artificial
  Synthetic
tattgcgatgctcttcaccga

SEQ ID NO: 4 - DNA, 20 bp, Artificial
  Synthetic
cgtttgtgatcctggctggt

SEQ ID NO: 5 - DNA, 20 bp, Artificial
  Synthetic
gagagttgtgcaccacagga

SEQ ID NO: 6 - DNA, 19 bp, Artificial
  Synthetic
cagcacttcgtcggtctgg

SEQ ID NO: 7 - DNA, 20 bp, Artificial
  Synthetic
cagttacttgccgaacgcag

SEQ ID NO: 8 - DNA, 20 bp, Artificial
  Synthetic
ttttgtgtccagtgaccgct

SEQ ID NO: 9 - DNA, 20 bp, Artificial
  Synthetic
attgacgcgggtaatgctct

SEQ ID NO: 10 - DNA, 20 bp, Artificial
  Synthetic
ccgcgacatcgtaatcctca

SEQ ID NO: 11 - DNA, 20 bp, Artificial
  Synthetic
tggtgcatggtgaagtgaca

SEQ ID NO: 12 - DNA, 20 bp, Artificial
  Synthetic
ccagcgtgtcatcagtcagt

SEQ ID NO: 13 - DNA, 20 bp, Artificial
  Synthetic
ggtggattccggcattgagt

SEQ ID NO: 14 - DNA, 20 bp, Artificial
  Synthetic
tgcggcacagttgcagtaat

SEQ ID NO: 15 - DNA, 20 bp, Artificial
  Synthetic
gatgtttcgcttggtggtcg